(19) United States
(12) Patent Application Publication
(10) Pub. No.: US 2026/0007734 A1
(43) Pub. Date: Jan. 8, 2026

(54) IMMUNOGENIC COMPOSITIONS FOR THE PREVENTION OF INFLUENZA A

(71) Applicant: Oxford University Innovation Limited, Oxford (GB)

(72) Inventors: Sunetra GUPTA, Oxford (Oxfordshire) (GB); Jose LOURENCO, Oxford (Oxfordshire) (GB)

(21) Appl. No.: 18/850,073

(22) PCT Filed: Mar. 29, 2023
(86) PCT No.: PCT/GB2023/050809

§ 371 Date: Sep. 24, 2024

(30) Foreign Application Priority Data
Mar. 29, 2022 (GB) 2204478.8

(51) Int. Cl. A61K 39/145; A61K 38/00; A61K 39/00; C07K 14/005; C12N 7/00

(52) U.S. Cl. CPC A61K 39/145; C07K 14/005; C12N 7/00; A61K 38/00; A61K 2039/5258; A61K 2039/572; C12N 2760/16122; C12N 2760/16123; C12N 2760/16134

(57) ABSTRACT

The present invention relates to polypeptides and immunogenic compositions, particularly vaccine compositions, for the prevention or treatment of influenza A. The invention also provides nucleic acid molecules and vectors encoding the polypeptides, and methods of using the compositions, nucleic acid molecules and vectors for the prevention or treatment of influenza A.

The present invention relates to polypeptides and immunogenic compositions, particularly vaccine compositions, for the prevention or treatment of influenza A. The invention also provides nucleic acid molecules and vectors encoding the polypeptides, and methods of using the compositions, nucleic acid molecules and vectors for the prevention or treatment of influenza A.

Seasonal influenza is a serious public health problem that causes severe illness and death. Worldwide, seasonal influenza is estimated to cause 3 to 5 million cases of severe illness and 250,000 to 500,000 deaths (Lozano et al. 2012). The demographics highest at risk of complications are children younger than 2 years of age, adults aged over 65, pregnant women, and people of any age with certain medical conditions such as diabetes or weakened immune systems (Mertz et al. 2013). It is estimated that a large proportion of child deaths in developing countries are associated with influenza. Seasonal influenza also causes high levels of workforce absenteeism and productivity losses.

Influenza pandemics occur sporadically when a distinct influenza strain from an animal reservoir begins to circulate widely in the human population. The most recent influenza pandemic occurred in 2009, which caused an increase in severe influenza illness and hospitalisation in individuals aged under 35 (Presonis et al., 2011; Manicassamy et al., 2010). The 1918 influenza pandemic was the most serious pandemic in recorded history, causing 50-100 million deaths. The emergence of a new pandemic influenza strain remains of concern.

The most effective way to prevent illness from influenza infection is vaccination. Currently, vaccination against influenza involves a trivalent or quatrivalent vaccine consisting of the most recent circulating strains of the H1N1 and H3N2 subtypes of influenza A and also includes one or two of influenza B strains (WHO, 2016). Due to the rapid antigenic evolution of influenza, the vaccine has to be constantly updated, and often due to time constrains, the wrong vaccine strains for the coming influenza season are chosen. For these reasons, the convention trivalent vaccine is estimated to have 10-60% efficacy and immunisation of at risk groups takes place annually (Treonor et al. 2012; Belongia et al. 2009).

Consequently, there are clear societal and economic benefits for improving the current influenza vaccines. This has been recognised by pharmaceutical companies, such as GSK and Pfizer, who are developing their own new influenza vaccines. Such approaches typically target epitopes that are under weak immune selection and therefore ‘immunorecessive’.

The influenza virus is currently conceptualised as containing (i) highly immunogenic (and protective) epitopes of high variability, as well as (ii) invariant epitopes of low immunogenicity. Together, these form the backbone of the theory of “antigenic drift” whereby the virus population slowly and incrementally acquires changes in the highly variable epitope regions requiring vaccines directed against these sites to be continuously updated, with the only other alternative being seen as the artificial boosting of immunity to invariant epitopes of low natural efficacy.

The inventors propose, by contrast, that the influenza virus also contains highly immunogenic epitopes of low variability and that universal vaccines may be constructed by identifying these protective epitopes. This idea is underpinned by an alternative theory of influenza evolution known as “antigenic thrift” in which viral dynamics are driven by pre-existing immunity to shared epitopes, but the existence of such epitopes has remained in doubt and their use in vaccination has never previously been mooted.

Using a combination of bioinformatics, structural and serological analyses, one epitope of limited variability that is under strong immune selection in the major influenza antigen, haemagglutinin (HA), has now been identified and characterised.

HA is the major surface antigen in influenza viruses. It binds sialic acid and initiates membrane fusion, leading to endocytosis. It is a trimeric protein typically 565/566 amino acids in length. Each monomer consists of a head domain and a stem domain.

The epitope which has now been identified is under strong immune selection and is therefore ‘immunodominant’. This has enabled the design of a new ‘universal’ influenza vaccine that protects against the majority of H1N1 influenza strains by targeting this epitope of limited variability.

Consequently, the vaccine of the current invention has a number of advantages over the conventional trivalent vaccine and other influenza vaccines in development. These advantages include:
(i) infection with circulating influenza strains will reinforce vaccine protection instead of potentially detracting from it;
(ii) the vaccine should be more immunogenic than other ‘universal’ vaccines in development, leading to lower thresholds of protection and greater longevity of protection;
(iii) it should only need to be administered between one and three times (i.e. a prime and a boost, or a prime and two boosts); and
(iv) the theoretical and experimental framework from which the vaccine has been derived suggests that the influenza virus is not likely to escape the protection conferred by the proposed vaccine.

It is therefore an object of the invention to provide polypeptides for use in an influenza vaccine composition which is capable of conferring protection against one or more influenza A subtypes, preferably against the H3 subtype.

In one embodiment, the invention provides a polypeptide which comprises a first region of contiguous amino acids, wherein:
(a) the amino acid sequence of the first region has at least 80% sequence identity to an influenza A haemagglutinin head domain; and
(b) the first region has all of the following specified amino acids at positions corresponding to the positions given in SEQ ID NO: 31:
position 155 is C
position 156 is I or K
position 157 is R
position 158 is G or R
position 159 is P or S
position 160 is a non-polar or uncharged amino acid or is D or K
position 161 is an uncharged amino acid or is K
position 162 is an uncharged amino acid
position 163 is F
position 164 is F
position 165 is S
position 166 is R
position 167 is L
position 168 is N and
position 169 is W.

In some embodiments of the invention, the amino acid sequence of the polypeptide does not comprise the amino acid sequence given in any one of SEQ ID NOs: 1-5.

Preferably, (a) the amino acid sequence of the first region of the polypeptide has at least 80% sequence identity to an influenza A subtype H1, H2, H3, H4, H5, H6, H7, H8, H9, H10, H11, H12, H13, H14, H15, H16, H17 or H18 haemagglutinin head domain; more preferably an influenza A subtype H4, H7, H10, H14 or H15 haemagglutinin head domain; and most preferably, the head domain as given in any one of SEQ ID NOs: 1-5.

In one preferred embodiment, (b) the first region of the polypeptide has all of the following specified amino acids at positions which correspond to the positions in SEQ ID NO: 31:
position 155 is C
position 156 is I or K
position 157 is R
position 158 is G or R
position 159 is P or S
position 160 is I, V, S, N, G, D or K
position 161 is S, N or K
position 162 is G or S
position 163 is F
position 164 is F
position 165 is S
position 166 is R
position 167 is L
position 168 is N and
position 169 is W.

In another preferred embodiment (“Omega”), (b) the first region of the polypeptide has the following specified amino acids at positions which correspond to the positions in SEQ ID NO:31:
position 155 is C
position 156 is I
position 157 is R
position 158 is G or R, preferably R
position 159 is S
position 160 is N or S, preferably S
position 161 is N or S, preferably S
position 162 is S
position 163 is F
position 164 is F
position 165 is S
position 166 is R
position 167 is L
position 168 is N and
position 169 is W.

In a particularly preferred embodiment (“Omega”), (b) the first region of the polypeptide has the following specified amino acids at positions which correspond to the positions in SEQ ID NO:31:
position 155 is C
position 156 is I
position 157 is R
position 158 is R
position 159 is S
position 160 is S
position 161 is S
position 162 is S
position 163 is F
position 164 is F
position 165 is S
position 166 is R
position 167 is L
position 168 is N and
position 169 is W.

More preferably, the first region of one polypeptide comprises or consists of the amino acid sequence given in SEQ ID NO: 21.

In a particularly preferred embodiment (“Tau”), (b) the first region of the polypeptide has the following specified amino acids at positions which correspond to the positions in SEQ ID NO:31:
position 155 is C
position 156 is K
position 157 is R
position 158 is R
position 159 is S
position 160 is N
position 161 is N
position 162 is S
position 163 is F
position 164 is F
position 165 is S
position 166 is R
position 167 is L
position 168 is N and
position 169 is W.

More preferably, the first region of one polypeptide comprises or consists of the amino acid sequence given in SEQ ID NO: 22.

In another preferred embodiment (“Sigma”), (b) the first region of the polypeptide has the following specified amino acids at positions which correspond to the positions in SEQ ID NO:31:
position 155 is C
position 156 is K
position 157 is R
position 158 is R
position 159 is S
position 160 is D, I, N or V, preferably N
position 161 is K
position 162 is S
position 163 is F
position 164 is F
position 165 is S
position 166 is R
position 167 is L
position 168 is N and
position 169 is W.

In a particularly preferred embodiment (“Sigma”), (b) the first region of the polypeptide has the following specified amino acids at positions which correspond to the positions in SEQ ID NO:31:
position 155 is C
position 156 is K
position 157 is R
position 158 is R
position 159 is S
position 160 is N
position 161 is K
position 162 is S
position 163 is F
position 164 is F
position 165 is S
position 166 is R
position 167 is L
position 168 is N and
position 169 is W.

More preferably, the first region of one polypeptide comprises or consists of the amino acid sequence given in SEQ ID NO: 23.

In a particularly preferred embodiment (“Rho”), (b) the first region of the polypeptide has the following specified amino acids at positions which correspond to the positions in SEQ ID NO:31:
position 155 is C
position 156 is K
position 157 is R
position 158 is G
position 159 is S
position 160 is V
position 161 is K
position 162 is S
position 163 is F
position 164 is F
position 165 is S
position 166 is R
position 167 is L
position 168 is N and
position 169 is W.

More preferably, the first region of one polypeptide comprises or consists of the amino acid sequence given in SEQ ID NO: 24.

In another preferred embodiment (“Mu”), (b) the first region of the polypeptide has the following specified amino acids at positions which correspond to the positions in SEQ ID NO: 31:
position 155 is C
position 156 is K
position 157 is R
position 158 is G
position 159 is P or S, preferably S
position 160 is G or V, preferably V
position 161 is N or S, preferably N
position 162 is G or S, preferably S
position 163 is F
position 164 is F
position 165 is S
position 166 is R
position 167 is L
position 168 is N and
position 169 is W.

In a particularly preferred embodiment (“Mu”), (b) the first region of the polypeptide has the following specified amino acids at positions which correspond to the positions in SEQ ID NO: 31:
position 155 is C
position 156 is K
position 157 is R
position 158 is G
position 159 is S
position 160 is V
position 161 is N
position 162 is S
position 163 is F
position 164 is F
position 165 is S
position 166 is R
position 167 is L
position 168 is N and
position 169 is W.

More preferably, the first region of one polypeptide comprises or consists of the amino acid sequence given in SEQ ID NO: 25.

In another preferred embodiment (“Theta”), (b) the first region of the polypeptide has the following specified amino acids at positions which correspond to the positions in SEQ ID NO: 31:
position 155 is C
position 156 is K
position 157 is R
position 158 is G
position 159 is P or S
position 160 is D
position 161 is K, N or S, preferably N
position 162 is G or S, preferably S
position 163 is F
position 164 is F
position 165 is S
position 166 is R
position 167 is L
position 168 is N and
position 169 is W.

In a particularly preferred embodiment (“Theta”), (b) the first region of the polypeptide has the following specified amino acids at positions which correspond to the positions in SEQ ID NO: 31:
position 155 is C
position 156 is K
position 157 is R
position 158 is G
position 159 is S
position 160 is D
position 161 is N
position 162 is S
position 163 is F
position 164 is F
position 165 is S
position 166 is R
position 167 is L
position 168 is N and
position 169 is W.

More preferably, the first region of one polypeptide comprises or consists of the amino acid sequence given in SEQ ID NO: 26.

In one embodiment, the invention provides a polypeptide (“MAIZ-short”) which comprises a first region of contiguous amino acids, wherein:
(a) the amino acid sequence of the first region has at least 80% sequence identity to an influenza A haemagglutinin head domain; and
(b) the first region has all of the following specified amino acids at positions corresponding to the positions given in SEQ ID NO: 31:
position 156 is I or K
position 157 is R
position 158 is G or R
position 159 is P or S
position 160 is S, N, I, V, D or G
position 161 is S, N or K.

In a particularly-preferred embodiment (“Ruby1”), (b) the first region of the polypeptide has the following specified amino acids at positions which correspond to the positions in SEQ ID NO: 31:
position 156 is |
position 157 is R
position 158 is R
position 159 is S
position 160 is S, N, I or D
position 161 S or N.

In a particularly-preferred embodiment (“Ruby2”), (b) the first region of the polypeptide has the following specified amino acids at positions which correspond to the positions in SEQ ID NO: 31:
position 156 is K
position 157 is R
position 158 is R
position 159 is S
position 160 S, N, I or D
position 161 S or N.

In a particularly preferred embodiment (“Emerald”), (b) the first region of the polypeptide has the following specified amino acids at positions which correspond to the positions in SEQ ID NO: 31:
position 156 is K
position 157 is R
position 158 is G
position 159 is S
position 160 is V
position 161 is Kor N.

In a particularly preferred embodiment (“Sapphire1”), (b) the first region of the polypeptide has the following specified amino acids at positions which correspond to the positions in SEQ ID NO: 31:
position 156 is K
position 157 is R
position 158 is G
position 159 is P or S
position 160 is G
position 161 is N, S or K.

In a particularly preferred embodiment (“Sapphire2”), (b) the first region of the polypeptide has the following specified amino acids at positions which correspond to the positions in SEQ ID NO: 31:
position 156 is K
position 157 is R
position 158 is G
position 159 is S
position 160 is D
position 161 is N, S or K.

In other embodiment, the invention provides a polypeptide (“MEIZ”) which comprises a first region of contiguous amino acids, wherein:
(a) the amino acid sequence of the first region has at least 80% sequence identity to an influenza A haemagglutinin head domain; and
(b) the first region has all of the following specified amino acids at positions corresponding to the positions given in SEQ ID NO: 31:
position 149 is N, S or D
position 150 is G
position 151 is T, G, E, D or K
position 152 is S
position 153 is S, N or Y
position 154 is A
position 155 is C.

In one preferred embodiment, (b) the first region of the polypeptide (“Topaz1”) has the following specified amino acids at positions which correspond to the positions in SEQ ID NO: 31:
position 149 is N, S or D
position 150 is G
position 151 is T or G
position 152 is S
position 153 is S, N or Y
position 154 is A
position 155 is C.

In one preferred embodiment, (b) the first region of the polypeptide (“Topaz2”) has the following specified amino acids at positions which correspond to the positions in SEQ ID NO: 31:
position 149 is N, S or D
position 150 is G
position 151 is E or D
position 152 is S
position 153 is S, N or Y
position 154 is A
position 155 is C.

In one preferred embodiment, (b) the first region of the polypeptide (“Opal”) has the following specified amino acids at positions which correspond to the positions in SEQ ID NO: 31:
position 149 is D
position 150 is G
position 151 is K
position 152 is S
position 153 is Y
position 154 is A
position 155 is C.

In a particularly-preferred embodiment, the invention provides a polypeptide which comprises a first region of contiguous amino acids, wherein:
(a) the amino acid sequence of the first region has at least 80% sequence identity to an influenza A haemagglutinin head domain; and
(b) the first region comprises the following specified amino acids at positions corresponding to positions 143-169 in SEQ ID NO: 31:

OMEGA (TOPAZ-RUBY1):
(SEQ ID NO: 60)
WTGVTQNGTSSACIRRSSSSFFSRLNW
RHO2 (OPAL-EMERALD):
(SEQ ID NO: 61)
WTGVAQDGKSYACKRGSVNSFFSRLNW
THETA (TOPAZ-SAPPHIRE2):
(SEQ ID NO: 62)
WTGVTQNGGSYACKRGPDNSFFSRLNW
RHO (TOPAZ2-EMERALD):
(SEQ ID NO: 63)
WTGVAQSGESYACKRGSVKSFFSRLNW
MU (TOPAZ-SAPPHIRE):
(SEQ ID NO: 64)
WTGVTQNGGSNACKRGPGSGFFSRLNW
SIGMA (TOPAZ-RUBY2):
(SEQ ID NO: 65)
WTGVTQNGTSSACKRRRNSGFFSRLNW

Preferably, the amino acid sequence of the first region of the polypeptide has at least 80% or 90% sequence identity to an influenza A subtype H4, H7 or H10 haemagglutinin head domain.

The invention further provides a composition comprising 1, 2, 3, 4, 5 or 6 (preferably 3) different polypeptides selected from the above polypeptides.

In yet another embodiment, the invention provides a polypeptide which comprises a first region of contiguous amino acids, wherein:
(a) the amino acid sequence of the first region has at least 80% sequence identity to an influenza A haemagglutinin head domain; and
(b) the first region has the following specified amino acids at positions corresponding to the positions given in SEQ ID NO: 31:
position 142 is an uncharged amino acid or is D
position 143 is W
position 144 is A or T
position 145 is G
position 146 is V
position 187 is K or N
position 188 is a negatively charged amino acid or is G
position 189 is an uncharged amino acid or is K
position 190 is F or S
position 191 is D
position 192 is K
position 193 is L
position 194 is Y
position 195 is I
position 196 is W
position 197 is G
position 198 is V
position 227 is Q
position 228 is A or T
position 229 is a non-polar amino acid
position 256 is G
position 257 is D
position 259 is L
position 260 is a non-polar amino acid
position 261 is I
position 262 is N
position 263 is S and
position 265 is G.

In some embodiments of the invention, the amino acid sequence of the polypeptide does not comprise the amino acid sequence given in any one of SEQ ID NOs: 1-5.

In one preferred embodiment, (b) the first region of the polypeptide has the following specified amino acids at positions which correspond to the positions in SEQ ID NO:31:
position 142 is D, N or T
position 143 is W
position 144 is A or T
position 145 is G
position 146 is V
position 187 is K or N
position 188 is D, E or G
position 189 is K, N or Q
position 190 is F or S
position 191 is D
position 192 is K
position 193 is L
position 194 is Y
position 195 is I
position 196 is W
position 197 is G
position 198 is V
position 227 is Q
position 228 is A or T
position 229 is I or V
position 256 is G
position 257 is D
position 259 is L
position 260 is L or V
position 261 is |
position 262 is N
position 263 is S and
position 265 is G.

In a preferred embodiment (“Alpha”), (b) the first region of the polypeptide has the following specified amino acids at positions which correspond to the positions in SEQ ID NO: 31:
position 142 is N or T, preferably N
position 143 is W
position 144 is A or T, preferably T
position 145 is G
position 146 is V
position 187 is K or N, preferably K
position 188 is D or E, preferably E
position 189 is N or Q, preferably Q
position 190 is F or S, preferably F
position 191 is D
position 192 is K
position 193 is L
position 194 is Y
position 195 is I
position 196 is W
position 197 is G
position 198 is V
position 227 is Q
position 228 is A or T, preferably A
position 229 is I or V, preferably V
position 256 is G
position 257 is D
position 259 is L
position 260 is L or V, preferably L
position 261 is I
position 262 is N
position 263 is S and
position 265 is G.

In a particularly preferred embodiment (“Alpha”), (b) the first region of the polypeptide has the following specified amino acids at positions which correspond to the positions in SEQ ID NO:31:
position 142 is N
position 143 is W
position 144 is T
position 145 is G
position 146 is V
position 187 is K
position 188 is E
position 189 is Q
position 190 is F
position 191 is D
position 192 is K
position 193 is L
position 194 is Y
position 195 is I
position 196 is W
position 197 is G
position 198 is V
position 227 is Q
position 228 is A
position 229 is V
position 256 is G
position 257 is D
position 259 is L
position 260 is L
position 261 is I
position 262 is N
position 263 is S and
position 265 is G.

More preferably, the first region of one polypeptide comprises or consists of the amino acid sequence given in SEQ ID NO: 27.

In one preferred embodiment (“Beta”), (b) the first region of the polypeptide has the following specified amino acids at positions which correspond to the positions in SEQ ID NO: 31:
position 142 is D or N, preferably N
position 143 is W
position 144 is T
position 145 is G
position 146 is V
position 187 is N
position 188 is D or E, preferably E
position 189 is K
position 190 is F
position 191 is D
position 192 is K
position 193 is L
position 194 is Y
position 195 is I
position 196 is W
position 197 is G
position 198 is V
position 227 is Q
position 228 is T
position 229 is V
position 256 is G
position 257 is D
position 259 is L
position 260 is L
position 261 is I
position 262 is N
position 263 is S and
position 265 is G.

In a particularly preferred embodiment (“Beta”), (b) the first region of the polypeptide has the following specified amino acids at positions which correspond to the positions in SEQ ID NO:31:
position 142 is N
position 143 is W
position 144 is T
position 145 is G
position 146 is V
position 187 is N
position 188 is E
position 189 is K
position 190 is F
position 191 is D
position 192 is K
position 193 is L
position 194 is Y
position 195 is I
position 196 is W
position 197 is G
position 198 is V
position 227 is Q
position 228 is T
position 229 is V
position 256 is G
position 257 is D
position 259 is L
position 260 is L
position 261 is I
position 262 is N
position 263 is S and
position 265 is G.

Most preferably, the first region of one polypeptide comprises or consists of the amino acid sequence given in SEQ ID NO: 28.

In a particularly preferred embodiment (“Gamma”), (b) the first region of the polypeptide has the following specified amino acids at positions which correspond to the positions in SEQ ID NO:31:
position 142 is N
position 143 is W
position 144 is T
position 145 is G
position 146 is V
position 187 is N
position 188 is G
position 189 is K
position 190 is F
position 191 is D
position 192 is K
position 193 is L
position 194 is Y
position 195 is I
position 196 is W
position 197 is G
position 198 is V
position 227 is Q
position 228 is T
position 229 is V
position 256 is G
position 257 is D
position 259 is L
position 260 is L
position 261 is I
position 262 is N
position 263 is S and
position 265 is G.

Preferably, the first region of one polypeptide comprises or consists of the amino acid sequence given in SEQ ID NO: 29.

In a particularly preferred embodiment (“Delta”), (b) the first region of the polypeptide has the following specified amino acids at positions which correspond to the positions in SEQ ID NO:31:
position 142 is N
position 143 is W
position 144 is T
position 145 is G
position 146 is V
position 187 is N
position 188 is G
position 189 is N
position 190 is F
position 191 is D
position 192 is K
position 193 is L
position 194 is Y
position 195 is I
position 196 is W
position 197 is G
position 198 is V
position 227 is Q
position 228 is T
position 229 is I
position 256 is G
position 257 is D
position 259 is L
position 260 is L
position 261 is |
position 262 is N
position 263 is S and
position 265 is G.

More preferably, the first region of one polypeptide comprises or consists of the amino acid sequence given in SEQ ID NO: 30.

In yet another embodiment, the invention provides a polypeptide (“INDY-short”) which comprises a first region of contiguous amino acids, wherein:
(a) the amino acid sequence of the first region has at least 80% sequence identity to an influenza A haemagglutinin head domain; and
(b) the first region has the following specified amino acids at positions corresponding to the positions given in SEQ ID NO: 31:
position 187 is K or N
position 188 is E, D or G
position 189 is Q, N, K
position 190 is F or S, preferably F
position 191 is D
position 192 is K
position 193 is L
position 194 is Y
position 195 is I
position 196 is W
position 197 is G
position 198 is V.

In a particularly preferred embodiment (“Alpha-short”), (b) the first region of the polypeptide has the following specified amino acids at positions which correspond to the positions in SEQ ID NO:31:
position 187 is N
position 188 is E or D
position 189 is Q or N
position 190 is F or S, preferably F
position 191 is D
position 192 is K
position 193 is L
position 194 is Y
position 195 is I
position 196 is W
position 197 is G
position 198 is V.

In a particularly preferred embodiment (“Beta-short”), (b) the first region of the polypeptide has the following specified amino acids at positions which correspond to the positions in SEQ ID NO:31:
position 187 is N
position 188 is E or D
position 189 is K
position 190 is F
position 191 is D
position 192 is K
position 193 is L
position 194 is Y
position 195 is I
position 196 is W
position 197 is G
position 198 is V.

In a particularly preferred embodiment (“Gamma-short”), (b) the first region of the polypeptide has the following specified amino acids at positions which correspond to the positions in SEQ ID NO:31:
position 187 is N
position 188 is G
position 189 is K
position 190 is F
position 191 is D
position 192 is K
position 193 is L
position 194 is Y
position 195 is I
position 196 is W
position 197 is G
position 198 is V.

In a particularly preferred embodiment (“Delta-short”), (b) the first region of the polypeptide has the following specified amino acids at positions which correspond to the positions in SEQ ID NO:31:
position 187 is N
position 188 is G
position 189 is N
position 190 is F
position 191 is D
position 192 is K
position 193 is L
position 194 is Y
position 195 is I
position 196 is W
position 197 is G
position 198 is V.

In a particularly preferred embodiment (“Omicron-short”), (b) the first region of the polypeptide has the following specified amino acids at positions which correspond to the positions in SEQ ID NO:31:
position 187 is K
position 188 is E
position 189 is Q
position 190 is F
position 191 is D
position 192 is K
position 193 is L
position 194 is Y
position 195 is I
position 196 is W
position 197 is G
position 198 is V.

Preferably, (a) the amino acid sequence of the first region of the polypeptide has at least 80% sequence identity to an influenza A subtype H1, H2, H3, H4, H5, H6, H7, H8, H9, H10, H11, H12, H13, H14, H15, H16, H17 or H18 haemagglutinin head domain; more preferably to an influenza A subtype H4, H7, H10, H14 or H15 haemagglutinin head domain; and most preferably, to the head domain as given in any one of SEQ ID NOs: 1-5.

The invention also provides a composition comprising one or more (e.g. 2, 3, 4, 5, 6, 7, 8, 9 or 10) different polypeptides of the invention, optionally together with one or more pharmaceutically-acceptable carriers, adjuvants, excipients or diluents.

In some embodiments, the composition comprises:
(i) one or more or all polypeptides having amino acid sequences selected from the group consisting of SEQ ID NOs: 21-26; and/or
(ii) one or more or all polypeptides having amino acid sequences selected from the group consisting of SEQ ID NOs: 27-30.
(iii) one or more or all polypeptides having amino acid sequences selected from the group consisting of SEQ ID NOs: 21-30.

In a preferred embodiment, the invention provides an immunogenic composition comprising a plurality of polypeptides, wherein:
(a) the plurality of polypeptides are polypeptides having amino acid sequences set forth in each of SEQ ID NOs: 21-26;
(b) the plurality of polypeptides are polypeptides having amino acid sequences set forth in each of SEQ ID NOs: 27-30; or
(c) the plurality of polypeptides are polypeptides having amino acid sequences set forth in each of SEQ ID NOs: 21-30.

The composition is preferably an immunogenic composition (e.g. a vaccine composition), wherein the composition is capable of inducing antibodies in a subject against an influenza A virus.

The invention also provides nucleic acids molecules (preferably DNA molecules and mRNAs) coding for such polypeptides, preferably wherein the DNA molecule is a vector or plasmid.

LIST OF SEQUENCES

| | |
|---|---|
| SEQ ID NO: | Sequence |

Head domain sequences

| | |
|---|---|
| 1 | Influenza A subtype H4 haemagglutinin head domain, amino acid |
| 2 | Influenza A subtype H7 haemagglutinin head domain, amino acid |
| 3 | Influenza A subtype H10 haemagglutinin head domain, amino acid |
| 4 | Influenza A subtype H14 haemagglutinin head domain, amino acid |
| 5 | Influenza A subtype H15 haemagglutinin head domain, amino acid |
| 6 | Influenza A subtype H4 haemagglutinin head domain, nucleotide |
| 7 | Influenza A subtype H7 haemagglutinin head domain, nucleotide |
| 8 | Influenza A subtype H10 haemagglutinin head domain, nucleotide |
| 9 | Influenza A subtype H14 haemagglutinin head domain, nucleotide |
| 10 | Influenza A subtype H15 haemagglutinin head domain, nucleotide |

Full haemagglutinin sequences

| | |
|---|---|
| 11 | Influenza A subtype H4 full haemagglutinin sequence, amino acid |
| 12 | Influenza A subtype H7 full haemagglutinin sequence, amino acid |
| 13 | Influenza A subtype H10 full haemagglutinin sequence, amino acid |
| 14 | Influenza A subtype H14 full haemagglutinin sequence, amino acid |
| 15 | Influenza A subtype H15 full haemagglutinin sequence, amino acid |
| 16 | Influenza A subtype H4 full haemagglutinin sequence, nucleotide |
| 17 | Influenza A subtype H7 full haemagglutinin sequence, nucleotide |
| 18 | Influenza A subtype H10 full haemagglutinin sequence, nucleotide |
| 19 | Influenza A subtype H14 full haemagglutinin sequence, nucleotide |
| 20 | Influenza A subtype H15 full haemagglutinin sequence, nucleotide |

Epitope 1 (“MAIZ”) sequences in preferred scaffolds

| | |
|---|---|
| 21 | Omega epitope in influenza A subtype H4 haemagglutinin head |
| | domain, amino acid |
| 22 | Tau epitope in influenza A subtype H4 haemagglutinin head domain, |
| | amino acid |
| 23 | Sigma epitope in influenza A subtype H4 haemagglutinin head |
| | domain, amino acid |
| 24 | Rho epitope in influenza A subtype H4 haemagglutinin head domain, |
| | amino acid |
| 25 | Mu epitope in influenza A subtype H4 haemagglutinin head domain, |
| | amino acid |
| 26 | Theta epitope in influenza A subtype H4 haemagglutinin head domain, |
| | amino acid |

Epitope 2 (“INDY”) sequences in preferred scaffolds

| | |
|---|---|
| 27 | Alpha epitope in influenza A subtype H4 haemagglutinin head domain, |
| | amino acid |
| 28 | Beta epitope in influenza A subtype H4 haemagglutinin head domain, |
| | amino acid |
| 29 | Gamma epitope in influenza A subtype H4 haemagglutinin head |
| | domain, amino acid |
| 30 | Delta epitope in influenza A subtype H4 haemagglutinin head domain, |
| | amino acid |

Reference sequence

| | |
|---|---|
| 31 | Influenza A H3N2 haemagglutinin head domain, amino acid |

mRNA vaccine sequences

| | |
|---|---|
| 32 | Omega epitope in influenza A subtype H4 haemagglutinin head |
| | domain, nucleotide sequence |
| 33 | Omega epitope in influenza A subtype H7 haemagglutinin head |
| | domain, nucleotide sequence |
| 34 | Omega epitope in influenza A subtype H10 haemagglutinin head |
| | domain, nucleotide sequence |
| 35 | Omega epitope in influenza A subtype H15 haemagglutinin head |
| | domain, nucleotide sequence |
| 36 | Mu epitope in influenza A subtype H4 haemagglutinin head domain, |
| | nucleotide sequence |
| 37 | Mu epitope in influenza A subtype H7 haemagglutinin head domain, |
| | nucleotide sequence |
| 38 | Mu epitope in influenza A subtype H10 haemagglutinin head domain, |
| | nucleotide sequence |
| 39 | Mu epitope in influenza A subtype H15 haemagglutinin head domain, |
| | nucleotide sequence |
| 40 | Theta epitope in influenza A subtype H4 haemagglutinin head domain, |
| | nucleotide sequence |
| 41 | Theta epitope in influenza A subtype H7 haemagglutinin head domain, |
| | nucleotide sequence |
| 42 | Theta epitope in influenza A subtype H10 haemagglutinin head |
| | domain, nucleotide sequence |
| 43 | Theta epitope in influenza A subtype H15 haemagglutinin head |
| | domain, nucleotide sequence |
| 44 | Rho epitope in influenza A subtype H4 haemagglutinin head domain, |
| | nucleotide sequence |
| 45 | Rho epitope in influenza A subtype H7 haemagglutinin head domain, |
| | nucleotide sequence |
| 46 | Rho epitope in influenza A subtype H10 haemagglutinin head domain, |
| | nucleotide sequence |
| 47 | Rho epitope in influenza A subtype H15 haemagglutinin head domain, |
| | nucleotide sequence |
| 48 | Sigma epitope in influenza A subtype H4 haemagglutinin head |
| | domain, nucleotide sequence |
| 49 | Sigma epitope in influenza A subtype H7 haemagglutinin head |
| | domain, nucleotide sequence |
| 50 | Sigma epitope in influenza A subtype H10 haemagglutinin head |
| | domain, nucleotide sequence |
| 51 | Sigma epitope in influenza A subtype H15 haemagglutinin head |
| | domain, nucleotide sequence |
| 52 | Tau epitope in influenza A subtype H4 haemagglutinin head domain, |
| | nucleotide sequence |
| 53 | Tau epitope in influenza A subtype H7 haemagglutinin head domain, |
| | nucleotide sequence |
| 54 | Tau epitope in influenza A subtype H10 haemagglutinin head domain, |
| | nucleotide sequence |
| 55 | Tau epitope in influenza A subtype H15 haemagglutinin head domain, |
| | nucleotide sequence |
| 56 | Backbone influenza A subtype H4 haemagglutinin head domain, |
| | nucleotide sequence |
| 57 | Backbone influenza A subtype H7 haemagglutinin head domain, |
| | nucleotide sequence |
| 58 | Backbone influenza A subtype H10 haemagglutinin head domain, |
| | nucleotide sequence |
| 59 | Backbone influenza A subtype H15 haemagglutinin head domain, |
| | nucleotide sequence |

Preferred OREO polypeptides

| | |
|---|---|
| 60 | OMEGA (TOPAZ-RUBY1), amino acid sequence |
| 61 | RHO2 (OPAL-EMERALD), amino acid sequence |
| 62 | THETA (TOPAZ-SAPPHIRE2), amino acid sequence |
| 63 | RHO (TOPAZ2-EMERALD), amino acid sequence |
| 64 | MU (TOPAZ-SAPPHIRE), amino acid sequence |
| 65 | SIGMA (TOPAZ-RUBY2), amino acid sequence |

Each polypeptide of the invention independently comprises a first region of contiguous amino acids. This region is a contiguous stretch of amino acids which are covalently joined.

In one embodiment, the amino acid sequence of the first region has at least 80% sequence identity to an influenza A haemagglutinin (HA) head domain. The intention is that this first region adopts the conformation of an influenza A haemagglutinin head domain.

The haemagglutinin head domain may, for example, be from any influenza A subtype, e.g. H1, H2, H3, H4, H5, H6, H7, H8, H9, H10, H11, H12, H13, H14, H15, H16, H17 or H18. Preferably, the haemagglutinin head domain is from an influenza A H4, H7, H10, H14 or H15 subtype. In one embodiment, haemagglutinin head domain is from an influenza A H4 subtype. In one embodiment, haemagglutinin head domain is from an influenza A H7 subtype. In one embodiment, haemagglutinin head domain is from an influenza A H10 subtype. In one embodiment, haemagglutinin head domain is from an influenza A H14 subtype. In one embodiment, haemagglutinin head domain is from an influenza A H15 subtype. Preferably, the influenza A N subtype is N2.

5 Consensus amino acid sequences of the H4, H7, H10, H14 and H15 haemagglutinin polypeptides are given herein as SEQ ID NOs: 11-15, respectively. The amino acid sequence numbering used herein is based on the numbering given to the influenza A H3N2 haemagglutinin head domain as given in SEQ ID NO: 31.

The HA polypeptide comprises two regions: the HA1 region and the HA2 region. These regions are separated by a potential cleavage site. Cleavage of HA0 into HA1 and HA2 occurs between R/GLF and is performed by a protease. The cleavage sites stated above are all described as monobasic. In some H5 viruses, a polybasic cleavage site is present, and this differs from the monobasic sites by having has multiple arginine residues (R's) and/or lysine residues (K's) in the critical position basic position.

Further details of the cleavage sites may be found in Sun et al., Journal of Virology, Sept. 2010, Vol. 84, No. 17, p. 8683-8690.

The HA1 region comprises 1-60 amino acids of the stalk, followed by the head domain, and then additional stalk amino acids. The HA2 region comprises only stalk amino acids.

The head domain of haemagglutinin is defined as being between two cysteines within the HA1 region. The first cysteine is generally at position 68; the second cysteine is generally at position 293. In influenza A H4 haemagglutinins, these cysteines are at positions 64 and 291. In influenza A H7 haemagglutinins, these cysteines are at positions 63 and 293. In influenza A H10 haemagglutinins, these cysteines are at positions 64 and 292. In influenza A H14 haemagglutinins, these cysteines are at positions 59 and 291. In influenza A H15 haemagglutinins, these cysteines are at positions 67 and 303.

Consensus amino acid sequences of the H4, H7, H10, H14 and H15 haemagglutinin head domains are given herein as SEQ ID NOs: 1-5, respectively.

In some embodiments, the amino acid sequence of the first region has at least 80%, 85%, 90% or 95% sequence identity to SEQ ID NO: 1. In some embodiments, the amino acid sequence of the first region has at least 80%, 85%, 90% or 95% sequence identity to SEQ ID NO: 2. In some embodiments, the amino acid sequence of the first region has at least 80%, 85%, 90% or 95% sequence identity to SEQ ID NO: 3. In some embodiments, the amino acid sequence of the first region has at least 80%, 85%, 90% or 95% sequence identity SEQ ID NO: 4. In some embodiments, the amino acid sequence of the first region has at least 80%, 85%, 90% or 95% sequence identity SEQ ID NO: 5. In some embodiments, the amino acid sequence of the first region has less than 100% sequence identity with all of SEQ ID NOs: 1-5.

In some embodiments, the amino acid sequence of the first region has at least 65%, 70%, 75%, 80%, 85%, 90%, 95%, 98%, 99% or 100% sequence identity to an influenza A haemagglutinin head domain at positions other than those that correspond to positions 155-169 of SEQ ID NO: 31.

In some embodiments, the amino acid sequence of the first region has at least 65%, 70%, 75%, 80%, 85%, 90%, 95%, 98%, 99% or 100% sequence identity to an influenza A H4 haemagglutinin head domain (preferably of SEQ ID NO: 1) at positions other than those that correspond to positions 155-169 of SEQ ID NO: 31. In some embodiments, the amino acid sequence of the first region has at least 65%, 70%, 75%, 80%, 85%, 90%, 95%, 98%, 99% or 100% sequence identity to an influenza A H7 haemagglutinin head domain (preferably of SEQ ID NO: 2) at positions other than those that correspond to positions 155-169 of SEQ ID NO: 31. In some embodiments, the amino acid sequence of the first region has at least 65%, 70%, 75%, 80%, 85%, 90%, 95%, 98%, 99% or 100% sequence identity to an influenza A H10 haemagglutinin head domain (preferably of SEQ ID NO: 3) at positions other than those that correspond to positions 155-169 of SEQ ID NO: 31. In some embodiments, the amino acid sequence of the first region has at least 65%, 70%, 75%, 80%, 85%, 90%, 95%, 98%, 99% or 100% sequence identity to an influenza A H14 haemagglutinin head domain (preferably of SEQ ID NO: 4) at positions other than those that correspond to positions 155-169 of SEQ ID NO: 31. In some embodiments, the amino acid sequence of the first region has at least 65%, 70%, 75%, 80%, 85%, 90%, 95%, 98%, 99% or 100% sequence identity to an influenza A H15 haemagglutinin head domain (preferably of SEQ ID NO: 5) at positions other than those that correspond to positions 155-169 of SEQ ID NO: 31.

In some embodiments, the amino acid sequence of the first region has at least 65%, 70%, 75%, 80%, 85%, 90%, 95%, 98%, 99% or 100% sequence identity to an influenza A haemagglutinin head domain at positions other than those that correspond to positions 142-146, 187-198, 227-229, 256-257, 259-263 and 265 of SEQ ID NO: 31.

In some embodiments, the amino acid sequence of the first region has at least 65%, 70%, 75%, 80%, 85%, 90%, 95%, 98%, 99% or 100% sequence identity to an influenza A H4 haemagglutinin head domain (preferably of SEQ ID NO: 1) at positions other than those that correspond to positions 142-146, 187-198, 227-229, 256-257, 259-263 and 265 of SEQ ID NO: 31. In some embodiments, the amino acid sequence of the first region has at least 65%, 70%, 75%, 80%, 85%, 90%, 95%, 98%, 99% or 100% sequence identity to an influenza A H7 haemagglutinin head domain (preferably of SEQ ID NO: 2) at positions other than those that correspond to positions 142-146, 187-198, 227-229, 256-257, 259-263 and 265 of SEQ ID NO: 31. In some embodiments, the amino acid sequence of the first region has at least 65%, 70%, 75%, 80%, 85%, 90%, 95%, 98%, 99% or 100% sequence identity to an influenza A H10 haemagglutinin head domain (preferably of SEQ ID NO: 3) at positions other than those that correspond to positions 142-146, 187-198, 227-229, 256-257, 259-263 and 265 of SEQ ID NO: 31. In some embodiments, the amino acid sequence of the first region has at least 65%, 70%, 75%, 80%, 85%, 90%, 95%, 98%, 99% or 100% sequence identity to an influenza A H14 haemagglutinin head domain (preferably of SEQ ID NO: 4) at positions other than those that correspond to positions 142-146, 187-198, 227-229, 256-257, 259-263 and 265 of SEQ ID NO: 31. In some embodiments, the amino acid sequence of the first region has at least 65%, 70%, 75%, 80%, 85%, 90%, 95%, 98%, 99% or 100% sequence identity to an influenza A H15 haemagglutinin head domain (preferably of SEQ ID NO: 5) at positions other than those that correspond to positions 142-146, 187-198, 227-229, 256-257, 259-263 and 265 of SEQ ID NO: 31.

In some embodiments, each polypeptide independently additionally comprises one or more amino acids which are contiguously joined to the first region at the N- and/or C-termini.

The additional N-terminal amino acids are preferably a stretch of contiguous amino acids which are derived from a haemagglutinin N-terminal stalk region, preferably from a haemagglutinin N-terminal stalk region of an influenza A H subtype, most preferably a H4, H7, H10, H14 or H15 subtype. Preferably, 58-60 amino acids of a haemagglutinin N-terminal stalk region of an influenza A subtype are contiguously joined to the N-terminal of the first region in one or more of the polypeptides.

In some embodiments, the amino acid sequence of this stalk region has at least 65%, 70%, 75%, 80%, 85%, 90%, 95% or 100% sequence identity to:
(i) amino acids 1-59 of SEQ ID NO: 11,
(ii) amino acids 1-58 of SEQ ID NO: 12,
(iii) amino acids 1-58 of SEQ ID NO: 13, or
(iv) amino acids 1-58 of SEQ ID NO: 14.

The additional C-terminal amino acids, if present, are preferably a stretch of contiguous amino acids (e.g. 1-300, 1-200, 1-100, 1-50 or 1-10 amino acids) which are derived from the haemagglutinin C-terminal stalk region of an influenza A subtype, preferably H4, H7, H10, H14 or H15. Preferably, the stretch of contiguous amino acids is derived from the haemagglutinin stalk region of the same influenza A subtype from which the head region is derived. These C-terminal stalk amino acids are contiguously joined to the C-terminal of the first region in one or more of the polypeptides of the invention.

In some embodiments, one or more of the polypeptides of the invention do not comprise an influenza A subtype HA2 region. In some embodiments, one or more of the polypeptides of the invention do not comprise the HA2 region of SEQ ID NOs: 11-15.

In some preferred embodiments, one or more or all of the polypeptides of the invention independently comprise an influenza A HA1 domain comprising a first region as defined herein, most preferably an influenza A H4, H7, H10, H14 or H15 subtype HA1 domain comprising a first region as defined herein.

Preferably, the polypeptides are independently less than 600, more preferably less than 400 and most preferably less than 300 amino acids in length. Preferably, the polypeptides are independently 250-350, more preferably 280-300 amino acids in length, and most preferably 290-292 amino acids in length.

The first region of each polypeptide of the invention has specified amino acids at positions which correspond to the positions in SEQ ID NO: 31. The first region of the polypeptide of the invention has one or more or all amino acid substitutions at specified positions which correspond to positions in SEQ ID NO: 31. For example, the first region of the polypeptide may have 1, 2, 3, 4, 5, 6, 7, 8, 9, 10, 11, 12, 13, 14, 15, 16, 17, 18, 19, 20, 221, 22, 23, 24 or 25 of the specified amino acid substitutions. Preferably, the first region of the polypeptide has all of the specified 15 or all of the specified 28 amino acid substitutions.

As used herein, the term “positively charged amino acid” includes lysine, arginine and histidine. As used herein, the term “negatively charged amino acid” includes aspartic acid and glutamic acid. As used herein, the term “non-polar amino acid” includes alanine, valine, leucine, isoleucine, methionine, proline, phenylalanine and tryptophan. As used herein, the term “uncharged amino acid” includes asparagine, glutamine, glycine, serine, threonine, tyrosine and cysteine.

In some preferred embodiments, the amino acid sequences of the polypeptides of the invention independently comprise or consist of an amino acid sequence of SEQ ID NOS: 21-30.

The polypeptides of the invention may be produced using recombinant methodology. For example, such techniques are described in “Molecular Cloning: A Laboratory Manual” (Fourth Edition) Michael R. Green and Joseph Sambrook. Alternatively, the nucleotide sequence encoding the polypeptides may be produced by chemical synthesis. Such a nucleotide sequence may then be ligated into an appropriate vector for host cell transformation or transfection. The polypeptides may then be expressed in such host cells. For modifications of existing HA genes, CRISPR-based techniques may also be used, such as those described in “CRISPR-Cas: A Laboratory Manual” (2016), edited by Jennifer Doudna (University of California, Berkeley) and Prashant Mali (University of California, San Diego). TALENs-based techniques may also be used.

Alternatively, the polypeptides of the invention may be synthesised using standard chemical peptide synthesis techniques. Solid phase synthesis of peptides in which the C-terminal amino acid of the sequence is attached to an insoluble support followed by sequential addition of the remaining amino acids may, for example, be used.

The polypeptides of the present invention include isolated polypeptides, i.e. polypeptides that have been removed from their naturally-occurring environment, and recombinant polypeptides, chemically-synthesized polypeptides, and polypeptides which have been synthesized biologically by heterologous systems.

In some embodiments of the invention, the amino acid sequence of the polypeptide of the invention does not comprise the amino acid sequence given in any one of SEQ ID NOs: 1-5 or any other wild-type sequence. In some embodiments of the invention, the amino acid sequence of the polypeptide of the invention contains at least one modification (e.g. insertion, deletion or substitution) relative to the amino acid sequence given in any one of SEQ ID NOs: 1-5 or any other wild-type sequence.

In a further embodiment, the invention provides a nucleic acid molecule which codes for one or more polypeptides of the invention. Preferably, the nucleic acid molecule encodes one, two or more, preferably 1, 2, 3, 4, 5, 6, 7, 8, 9 or 10 of the polypeptides of the invention.

Preferred nucleotide sequences include those encoding SEQ ID NOs: 21-30 and 60-65 of the invention.

As used herein, the terms “nucleic acid sequence”, “nucleic acid molecule” and “polynucleotide” are used interchangeably and do not imply any length restriction. These include DNA (including cDNA) and RNA sequences. The nucleic acid molecules of the present invention include isolated nucleic acid molecules that have been removed from their naturally-occurring environment, recombinant or cloned DNA isolates, and chemically-synthesized analogues or analogues which have been synthesized biologically by heterologous systems.

The nucleic acid molecules of the present invention may be prepared by any means known in the art. For example, large amounts of the polynucleotides may be produced by replication in a suitable host cell. The natural or synthetic DNA fragments coding for a desired fragment may be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The nucleic acid molecules of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

The original (e.g. wild-type) codons in a nucleic acid molecule may be optimised for expression in a desired cell line, for example, using an online tool such as that available at http://genomes.urv.es/OPTIMIZER/. In one embodiment of the invention, therefore, the nucleic acid molecule is codon-optimized for expression in a host cell, preferably a human cell.

As used herein, the term “product of the invention” refers to the polypeptides of the invention, nucleic acids of the invention, vectors of the invention, particles of the invention and compositions of the invention, inter alia.

In all embodiments of this invention, the polypeptides of the invention may be replaced by mRNAs coding for those polypeptides. Hence the invention in particular provides a mRNA molecule coding for one of SEQ ID NOs: 21-30 or 60-65, preferably for one of SEQ ID NOs: 21-26 or 60-65. The person of skill in the art will readily be able to produce such mRNA molecules. Furthermore, examples of mRNA sequences of the invention are given herein in SEQ ID NOs: 32-55. The invention also extends to mRNA sequences having at least 90% or 95% sequence identity to the non-underlined region of one of SEQ ID NOs: 32-55, wherein the mRNA sequence has 100% sequence identity to the underlined region (epitope region) of the corresponding sequence.

The invention also provides a vector or plasmid comprising a nucleic acid molecule of the invention. Preferably, the vector is an expression vector. The vector and/or plasmid may comprise one or more regulatory sequences which are operably linked to the sequence which encodes the polypeptide, e.g. one or more enhancer, promoter and/or transcriptional terminator sequences.

In some embodiments, the vector is viral vector, e.g. a poxvirus vector. In other embodiments, the vector is an adenoviral vector or a Modified Vaccinia Ankara (MVA) viral vector. Preferably, the vector is a non-replicating vector.

Non-replicating poxviruses and adenoviruses represent groups of viruses which may be used as vectors for the delivery of genetic material into a target cell. Viral vectors serve as antigen delivery vehicles and also have the power to activate the innate immune system through binding cell surface molecules that recognise viral elements. A recombinant viral vector can be produced that carries nucleic acid encoding a given antigen. The viral vector can then be used to deliver the nucleic acid to a target cell, where the encoded antigen is produced by the target cell's own molecular machinery. As “non-self”, the produced antigen generates an immune response in the target subject.

The vector of the invention may be a non-replicating poxvirus vector. As used herein, a non-replicating (or replication-deficient) viral vector is a viral vector which lacks the ability to productively replicate following infection of a target cell. Thus, a non-replicating viral vector cannot produce copies of itself following infection of a target cell. Non-replicating viral vectors may therefore advantageously have an improved safety profile as compared to replication-competent viral vectors.

In one embodiment, the non-replicating poxvirus vector is selected from a Modified Vaccinia virus Ankara (MVA) vector, a NYVAC vaccinia virus vector, a canary-pox (ALVAC) vector, and a fowlpox (FPV) vector. MVA and NYVAC are both attenuated derivatives of vaccinia virus. Compared to vaccinia virus, MVA lacks approximately 26 of the approximately 200 open reading frames. In one embodiment, the non-replicating poxvirus vector is an MVA vector.

The vector of the invention may be an adenovirus vector. In one embodiment, the adenovirus vector is a non-replicating adenovirus vector (wherein non-replicating is defined as above). Adenoviruses can be rendered non-replicating by deletion of the EI or both the EI and E3 gene regions. Alternatively, an adenovirus may be rendered non-replicating by alteration of the EI or of the EI and E3 gene regions such that said gene regions are rendered non-functional. For example, a non-replicating adenovirus may lack a functional EI region or may lack functional EI and E3 gene regions. In this way the adenoviruses are rendered replication-incompetent in most mammalian cell lines and do not replicate in immunised mammals. Most preferably, both EI and E3 gene region deletions are present in the adenovirus, thus allowing a greater size of transgene to be inserted. This is particularly important to allow larger antigens to be expressed, or when multiple antigens are to be expressed in a single vector, or when a large promoter sequence, such as the CMV promoter, is used. Deletion of the E3 as well as the EI region is particularly favoured for recombinant Ad5 vectors. Optionally, the E4 region can also be engineered.

In one embodiment, the adenovirus vector is selected from a human adenovirus vector, a simian adenovirus vector, a group B adenovirus vector, a group C adenovirus vector, a group E adenovirus vector, an adenovirus 6 vector, a PanAd3 vector, an adenovirus C3 vector, a ChAdY25 vector, an AdC68 vector, and an Ad5 vector.

The viral vector of the invention, as described above, can be used to deliver a single antigen to a target cell. Advantageously, the viral vector of the invention can also be used to deliver multiple (different) antigens to a target cell.

In one embodiment, the vector of the invention further comprises a nucleic acid sequence encoding an adjuvant (for example, a cholera toxin, an E. coli lethal toxin, or a flagellin).

The nucleic acid sequence encoding a vector (as described above) may be generated by the use of any technique for manipulating and generating recombinant nucleic acid known in the art. In one aspect, the invention provides a method of making a vector (as described above), comprising providing a nucleic acid, wherein the nucleic acid comprises a nucleic acid molecule encoding a vector of the invention; transfecting a host cell with the nucleic acid molecule; culturing the host cell under conditions suitable for the propagation of the vector; and obtaining the vector from the host cell.

As used herein, “transfecting” may mean any non-viral method of introducing nucleic acid molecules into a cell. The nucleic acid molecule may be any nucleic acid molecule suitable for transfecting a host cell. Thus, in one embodiment, the nucleic acid molecule is a plasmid. The host cell may be any cell in which a vector (i.e. a non-replicating poxvirus vector or an adenovirus vector, as described above) may be grown. As used herein, “culturing the host cell under conditions suitable for the propagation of the vector” means using any cell culture conditions and techniques known in the art which are suitable for the chosen host cell, and which enable the vector to be produced in the host cell. As used herein, “obtaining the vector”, means using any technique known in the art that is suitable for separating the vector from the host cell. Thus, the host cells may be lysed to release the vector. The vector may subsequently be isolated and purified using any suitable method or methods known in the art.

The invention also provides a host cell comprising a nucleic acid molecule, vector or plasmid of the invention. Preferably, the host cell is a eukaryotic host cell. Examples of eukaryotic host cells include yeast and mammalian cells.

The host cell is preferably a cell in which a vector (e.g. a non-replicating poxvirus vector or an adenovirus vector, as described above) may be grown or propagated. The host cell may be selected from a 293 cell (also known as a HEK, or human embryonic kidney, cell), a CHO cell (Chinese Hamster Ovary), a CCL81.1 cell, a Vero cell, a HELA cell, a Per.C6 cell, a BHK cell (Baby Hamster Kidney), a primary CEF cell (Chicken Embryo Fibroblast), a duck embryo fibroblast cell, or a DF-1 cell. In other embodiments, the host cell is a human cell (e.g. an isolated human cell).

In a further embodiment, there is provided a virus-like particle (VLP) comprising one, two or more (e.g. 2, 3, 4, 5, 6, 7, 8, 9 or 10) polypeptides of the invention. The particle is preferably immunogenic. Virus-like particles resemble viruses, but are non-infectious because they do not contain any viral genetic material. The particles may also be described as multimeric lipoprotein particles. Once expressed in an appropriate system, these VLPs are able to assemble spontaneously into lipoprotein structures/particles composed of one or more monomers of said polypeptides.

The invention also provides a VLP wherein one, two or more (e.g. 3, 4, 5, 6, 7, 8, 9 or 10) polypeptides (preferably different polypeptides) of the invention are covalently attached to the VLP. For example, the polypeptides of the invention may be covalently attached to the VLP by using chemical cross-linkers, reactive unnatural amino acids or SpyTag/SpyCatcher reactions.

In a particularly preferred embodiment, there is provided an immunogenic composition comprising at least five different virus-like particles (VLPs), wherein each VLP independently comprises one or more homotrimers consisting of or comprised of polypeptides of SEQ ID NOs: 21-30, optionally together with one or more pharmaceutically-acceptable carriers, adjuvants, excipients or diluents, as a combined preparation in a form suitable for simultaneous, separate or sequential use for treating or preventing influenza A infection.

In one embodiment, the invention relates to an immunogenic composition. As used herein, the term “immunogenic” is intended to refer to the ability to elicit a specific immune response against an influenza A subtype. This response may, for example, be when a composition of the invention is administered at an appropriate dose and in an appropriate formulation which may include/require a suitable adjuvant. A booster comprising a dose similar or less than the original dose may be required to obtain the required immunogenic response. In particular, the immunogenic composition of the invention is capable of inducing antibodies (preferably neutralising antibodies) in a subject against influenza A virus. Preferably, the immunogenic composition of the invention is capable of providing protection in a subject against influenza A virus.

More preferably, the immunogenic composition of the invention is capable of inducing antibodies (preferably neutralising antibodies) in a subject against the H1N1 influenza A subtype.

The capability of a composition of the invention to induce neutralising antibodies in a subject (e.g. a human subject) may be tested by purifying sera from the blood of subjects to whom the composition has been administered.

Antibodies may be measured using ELISA or a pseudotype micro-neutralisation (pMN) assay. ELISA is the most sensitive of these two assays; it quantifies all antibodies. In contrast, the pMN is less sensitive, but it quantifies neutralising antibodies.

As used herein, the reference to “influenza” relates to influenza virus, preferably influenza A virus, more preferably influenza A virus H3 subtypes, and most preferably influenza A virus H3N2 subtypes.

The immunogenic composition comprises one, two or more polypeptides. The amino acid sequences of these two or more polypeptides are preferably different.

The composition may, for example, comprise 2, 3, 4, 5, 6, 7, 8, 9 or 10 different polypeptides.

In a preferred embodiment, the invention provides a composition comprising:
(i) a polypeptide comprising an omega epitope;
(ii) a polypeptide comprising a tau epitope;
(iii) a polypeptide comprising a sigma epitope;
(iv) a polypeptide comprising a rho epitope;
(v) a polypeptide comprising a mu epitope; and
(vi) a polypeptide comprising a theta epitope;
or mRNAs encoding the said polypeptides.

In another preferred embodiment, the invention provides a composition comprising:
(i) a polypeptide comprising an alpha epitope;
(ii) a polypeptide comprising a beta epitope;
(iii) a polypeptide comprising a gamma epitope; and
(iv) a polypeptide comprising a delta epitope;
or mRNAs encoding the said polypeptides.

The sequences of the invention are derived from or based upon the head domain of influenza A haemagglutinin proteins. The naturally-occurring haemagglutinin protein is a homo-trimer of three polypeptides. In a preferred composition of the invention, the composition comprises three polypeptides as defined herein which form a homo-trimer. The composition may comprise more than one (e.g. 2, 3, 4, 5 or 6) different homo-trimers of the polypeptides defined herein.

In another preferred embodiment, three polypeptides of the invention form a hetero-trimer in the composition. The composition may comprise more than one (e.g. 2, 3, 4, 5 or 6) different hetero-trimers of the polypeptides defined herein.

The invention also provides a composition comprising one, two or more polypeptides of the invention, one or more nucleic acid molecules of the invention, one or more vectors of the invention or a VLP of the invention, optionally together with one or more pharmaceutically-acceptable carriers, excipients or diluents.

The composition is preferably an immunogenic composition.

Substances suitable for use as pharmaceutically-acceptable carriers are known in the art. Non-limiting examples of pharmaceutically-acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long term storage.

Examples of buffering agents include, but are not limited to, sodium succinate (pH 6.5), and phosphate buffered saline (PBS; pH 7.4).

In addition to a pharmaceutically-acceptable carrier, the composition of the invention can be further combined with one or more of a salt, excipient, diluent, adjuvant, immunoregulatory agent and/or antimicrobial compound.

In one embodiment, the products of the invention may contain 5% to 95% of active ingredient (i.e. polypeptide, nucleic acid, vectors or VLPs), such as at least 10% or 25% of active ingredient, or at least 40% of active ingredient or at least 50%, 55%, 60%, 70% or 75% active ingredient.

The products of the invention may be administered in a manner compatible with the dosage formulation, and in such amount as will be prophylactically and/or therapeutically effective.

Administration of the products of the invention is generally by conventional routes, e.g. intravenous, subcutaneous, intraperitoneal, or mucosal routes. The administration may be by parenteral administration; for example, a subcutaneous or intramuscular injection.

Accordingly, the products of the invention may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules. The active ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the products of the invention may also contain minor amounts of auxiliary substances such as wetting or emulsifying agents, and/or pH buffering agents.

Additional formulations which are suitable for other modes of administration include oral formulations or formulations suitable for distribution as aerosols. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

It may be desired to direct the products of the present invention (as described above) to the respiratory system of a subject. Efficient transmission of a therapeutic/prophylactic composition or medicament to the site of infection in the lungs may be achieved by oral or intra-nasal administration.

Formulations for intranasal administration may be in the form of nasal droplets or a nasal spray. An intranasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

Alternatively, the therapeutic/prophylactic formulation or medicament may be an aerosol formulation. The aerosol formulation may take the form of a powder, suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

Preferably, the composition of the invention is a vaccine composition, e.g. suitable for parenteral administration, optionally together with one or more adjuvants.

As used herein, a vaccine is a formulation that, when administered to an animal subject such as a mammal (e.g. a human, bovine, porcine, ovine, caprine, equine, cervine, canine or feline subject; in particular a human subject), stimulates a protective immune response against an infectious disease. The immune response may be a humoral and/or a cell-mediated immune response. Thus, the vaccine may stimulate B cells and/or T cells.

Examples of suitable adjuvants include those which are selected from the group consisting of:
metal salts such as aluminium hydroxide or aluminium phosphate,
oil in water emulsions,
toll like receptors agonist, (such as toll like receptor 2 agonist, toll like receptor 3 agonist, toll like receptor 4 agonist, toll like receptor 7 agonist, toll like receptor 8 agonist and toll like receptor 9 agonist),
saponins, for example Quil A and its derivatives such as QS7 and/or QS21,
CpG containing oligonucleotides,
3D-MPL,
(2-deoxy-6-O-[2-deoxy-2-[(R)-3-dodecanoyloxytetra-decanoylamino]-4-o-phosphono-β-D-glucopyranosy]]-2-[(R)-3-hydroxytetradecanoylamino]-α-D-glucopyranosyldihydrogenphosphate),
DP (3S, 9 R)-3-[(R)-dodecanoyloxytetradecanoylamino]-4-oxo-5-aza-9 (R)-[(R)-3-hydroxytetradecanoylamino] decan-1, 10-diol, 1, 10-bis(dihydrogenophosphate), and
MP-Ac DP (3S-, 9R)-3-[(R)-dodecanoyloxytetradecanoylamino]-4-oxo-5-aza-9-[(R)-3-hydroxytetradecanoylamino] decan-1, 10-diol, 1-dihydrogenophosphate 10-(6-aminohexanoate),
or combinations thereof.

Preferably, the adjuvant is selected from the group comprising:
a saponin associated with a metallic salt, such as aluminium hydroxide or aluminium phosphate
3D-MPL, QS21 and a CpG oligonucleotide, for example as an oil in water formulation,
saponin in the form of a liposome, for example further comprise a sterol such as QS21 and sterol, and
ISCOM.

In some particularly preferred embodiments, the adjuvant comprises a saponin. Saponins are steroid or triterpenoid glycosides, which occur in many plant species. Saponin-based adjuvants act in part by stimulating the entry of antigen-presenting cells into the injection site and enhancing antigen presentation in the local lymph nodes. Preferably, the adjuvant comprises saponin, cholesterol and a phospholipid, e.g. ISCOM Matrix-M™ (Isconova, Novavax).

In Matrix-M, purified saponin fractions are mixed with synthetic cholesterol and a phospholipid to form stable particles than can be readily formulated with a variety of vaccine antigens. Matrix-M™ induces both a cell-mediated and an antibody mediated immune response.

In some other preferred embodiments, the adjuvant comprises a squalene-oil-in-water nano-emulsion emulsion, e.g. AddaVax™ (InvivoGen).

Squalene is an oil which is more readily metabolized than the paraffin oil used in Freund's adjuvants. Squalene oil-in-water emulsions are known to elicit both cellular (Th1) and humoral (Th2) immune responses. This class of adjuvants is believed to act through recruitment and activation of APC and stimulation of cytokines and chemokines production by macrophages and granulocytes.

The composition may further comprise a surfactant. Examples of suitable surfactants include Tween (such as Tween 20), briji and polyethylene glycol.

Vaccine preparation is generally described in New Trends and Developments in Vaccines, edited by Voller et al., University Park Press, Baltimore, Maryland, U.S.A., 1978. Encapsulation within liposomes is described, for example, by Fullerton, U.S. Pat. No. 4,235,877.

The amount of the polypeptide, nucleic acid molecule, vector, or particle of the present invention present in each vaccine dose is selected as an amount which induces an immunoprotective response without significant, adverse side effects in typical vaccines. Such amount will vary depending upon which specific immunogen is employed and whether or not the vaccine is adjuvanted. Generally, it is expected that each does will comprise 1-1000 μg of protein, for example 1-200 μg, such as 10-100 μg, and more particularly 10-40 μg. An optimal amount for a particular vaccine can be ascertained by standard studies involving observation of antibody titres and other responses in subjects. Following an initial vaccination, subjects will preferably receive a boost in about 4 weeks, followed by repeated boosts every six months for as long as a risk of infection exists. The immune response to the products of this invention is enhanced by the use of adjuvant and or an immunostimulant.

The amount of saponin for use in the adjuvants of the present invention may be in the region of 1-1000 μg per dose, generally 1-500 μg per dose, more such as 1-250 μg per dose, and more specifically between 1 to 100 μg per dose (e.g. 10, 20, 30, 40, 50, 60, 70, 80 or 90 μg per dose).

The invention also provides a combined preparation comprising two or more components selected from two or more polypeptides of the invention, two or more particles of the invention, two or more nucleic acids of the invention, two or more vectors of the invention and two or more compositions of the invention as a combined preparation in a form suitable for simultaneous, separate or sequential use, preferably for treating or preventing influenza A infection.

In yet another aspect, the invention provides an antibody against a polypeptide of the invention.

In yet further embodiments, the invention provides a polypeptide of the invention, a particle of the invention, a nucleic acid of the invention, a vector of the invention or a composition of the invention for use in therapy or for use as a medicament.

In a further aspect, the invention provides a polypeptide of the invention, a particle of the invention, a nucleic acid of the invention, a vector of the invention or a composition of the invention for use in a method of preventing or treating influenza infection in a subject. In a further aspect, the invention provides a polypeptide of the invention, a particle of the invention, a nucleic acid of the invention, a vector of the invention or a composition of the invention for use in a method of inducing a T-cell or B-cell response to an influenza antigen in a subject. In particular, a non-replicating poxvirus vector of the invention can be used to stimulate a protective immune response via the cell-mediated immune system. In one embodiment, the T-cell is a T-helper cell (Th-cell). In one embodiment, the T-cell is a Tₕ17-cell. In further embodiments, the invention provides the use of a polypeptide of the invention, a particle of the invention, a nucleic acid of the invention, a vector of the invention or a composition of the invention in the manufacture of a medicament for use in a method of preventing or treating an influenza infection in a subject. In further embodiments, the invention provides the use of a polypeptide of the invention, a particle of the invention, a nucleic acid of the invention, a vector of the invention or a composition of the invention in the manufacture of a medicament for use in a method of inducing a T cell or B-cell response to an influenza antigen in a subject.

The invention also provides a method of treating a subject susceptible to influenza infection comprising administering an effective amount of a polypeptide of the invention, a particle of the invention, a nucleic acid of the invention, a vector of the invention or a composition of the invention to the subject in need thereof. The invention also provides a method of inducing a T-cell or B-cell response to an influenza antigen in a subject comprising administering an effective amount of a polypeptide of the invention, a particle of the invention, a nucleic acid of the invention, a vector of the invention or a composition of the invention to the subject in need thereof.

A polypeptide of the invention, a particle of the invention, a nucleic acid of the invention, a vector of the invention or a composition of the invention may also be used in similar uses and methods to produce neutralising antibodies in vivo against influenza antigens.

Preferably, the influenza antigen is the haemagglutinin protein, more preferably, the HA1 or head domain of a haemagglutinin protein. Preferably, the influenza is an influenza A.

The efficacy of the uses and methods to treat/prevent influenza infection may be tested (e.g. by ELISA) by establishing the presence or absence of neutralising antibodies against influenza virus in the subject's blood.

Also provided is an immunogenic composition comprising two or more polypeptides, two or more nucleic acid molecules or two or more vectors or plasmids as defined herein as a combined preparation in a form suitable for simultaneous, separate or sequential use for the treatment or prevention of influenza, preferably influenza A, or for inducing a T-cell or B-cell response in a subject against an influenza virus, preferably an influenza A virus. The subject is preferably a mammal, more preferably a human.

As used herein, the term “preventing” includes preventing the initiation of influenza infection and/or reducing the severity of intensity of an influenza infection. Thus, “preventing” encompasses vaccination.

As used herein, the term “treating” embraces therapeutic and preventative/prophylactic measures (including post-exposure prophylaxis) and includes post-infection therapy and amelioration of an influenza infection. Each of the above-described methods and uses can comprise the step of administering to a subject an effective amount, such as a therapeutically effective amount, of a polypeptide of the invention, a particle of the invention, a nucleic acid of the invention, a vector of the invention or a composition of the invention.

As used herein, an effective amount is a dosage or amount that is sufficient to achieve a desired biological outcome. As used herein, a therapeutically effective amount is an amount which is effective, upon single or multiple dose administration to a subject (such as a mammalian subject, in particular a human subject) for treating, preventing, curing, delaying, reducing the severity of, ameliorating at least one symptom of a disorder or recurring disorder, or prolonging the survival of the subject beyond that expected in the absence of such treatment.

Accordingly, the quantity of active ingredient to be administered depends on the subject to be treated, capacity of the subject's immune system to generate a protective immune response, and the degree of protection required. Precise amounts of active ingredient required to be administered may depend on the judgement of the practitioner and may be particular to each subject. Administration to the subject can comprise administering to the subject a polypeptide of the invention, a particle of the invention, a nucleic acid of the invention, a vector of the invention or a composition of the invention (i.e. a product of the invention) wherein the product of the invention is sequentially administered multiple times (for example, wherein the composition is administered two, three or four times). Thus, in one embodiment, the subject is administered a polypeptide of the invention, a particle of the invention, a nucleic acid of the invention, a vector of the invention or a composition of the invention and is then administered the same product of the invention (or a substantially similar product) again at a different time.

In one embodiment, administration to a subject comprises administering a polypeptide of the invention, a particle of the invention, a nucleic acid of the invention, a vector of the invention or a composition of the invention to a subject, wherein said product of the invention is administered substantially prior to, simultaneously with, or subsequent to, another immunogenic composition.

The invention also extends to prime-boost regimes. For example, priming and/or boosting may be effected using one or more products of the invention. The products may be administered to a subject sequentially, simultaneously or separately.

A preferred prime-boost strategy of the invention provides a method of preventing or treating an influenza infection in a subject or of inducing a T-cell or B-cell response to an influenza antigen in a subject, the method comprising the steps of: (i) simultaneously, separately or sequentially administering an effective amount of one, two, three, four, five or more different polypeptides to a subject in need thereof,

Preferred influenza A haemagglutinin head domain sequences and first region substitutions are disclosed herein, mutatis mutandis.

The polypeptides may be in the form of a pharmaceutical composition, preferably a vaccine composition, optionally together with one or more pharmaceutically-acceptable carriers, diluents, excipients and adjuvants. Preferably, one or more of the polypeptides (as defined above) are in the form of one or more trimers. In some embodiments, the trimers are homo-trimers. In other embodiments, the trimers are hetero-trimers.

Preferably, the method comprises the additional steps of:
(ii) administering a boost with a second polypeptide to the subject; and optionally also
(iii) administering a boost with a third polypeptide to the subject, wherein the second and third polypeptides (as defined above) are preferably different to each other and preferably different to the first polypeptide.

Preferably, the method comprises the additional steps of:
(ii) administering a boost with a second trimer to the subject; and optionally also
(iii) administering a boost with a third trimer to the subject, wherein the second and third trimers are preferably different to each other and preferably different to the first trimer.

Preferably, the first, second and third polypeptides are independently selected from the group consisting of polypeptides comprising or consisting of SEQ ID NOs: 13-17.

Preferably, the first, second and third trimers independently consist of polypeptides comprising or consisting of SEQ ID NOs: 21-30.

In another preferred embodiment, the polypeptides or trimers are administered in the form of a VLP, i.e. a VLP is administered which comprises the polypeptide(s) trimer(s).

In another preferred embodiment, a nucleic acid molecule (preferably a vector) is administered to the subject, wherein the nucleic acid molecule encodes one or more of the polypeptides as defined above. Preferred vectors are discussed herein.

In one embodiment, the first and second products are administered as part of a prime-boost administration protocol. Thus, the first product may be administered to a subject as the “prime” and the second product subsequently administered to the same subject as the “boost”.

In one embodiment, the first product is an adenovirus vector of the invention prime, and the second product is a non-replicating poxvirus vector of the invention boost.

In one embodiment, each of the above-described methods further comprises the step of administration to the subject of a product of the invention.

In one embodiment, the polypeptide of the invention is administered separately from the administration of a viral vector of the invention. Preferably the polypeptide and a viral vector are administered sequentially, in any order. Thus, in one embodiment, the viral vector (“V”) and the polypeptide (“P”) may be administered in the order V-P, or in the order P-V.

In certain embodiments, the above-described methods further comprise the administration to the subject of an adjuvant. Adjuvant may be administered with any of the products of the invention.

The products of the invention may be given in a single dose schedule (i.e. the full dose is given at substantially one time). Alternatively, the products of the invention may be given in a multiple dose schedule. A multiple dose schedule is one in which a primary course of treatment (e.g. vaccination) may be with 1-6 separate doses, followed by other doses given at subsequent time intervals required to maintain and or reinforce the immune response, for example (for human subjects), at 1-4 months for a second dose, and if needed, a subsequent dose(s) after a further 1-4 months.

The dosage regimen will be determined, at least in part, by the need of the individual and be dependent upon the judgment of the practitioner (e.g. doctor or veterinarian).

Simultaneous administration means administration at (substantially) the same time.

Sequential administration of two or more products of the invention means that the products are administered at (substantially) different times, one after the other.

For example, sequential administration may encompass administration of two or more products of the invention at different times, wherein the different times are separated by a number of days (for example, 1, 2, 5, 10, 15, 20, 30, 60, 90, 100, 150 or 200 days).

For example, in one embodiment, the vaccine of the present invention may be administered as part of a ‘prime-boost’ vaccination regime.

In one embodiment, the products of the invention can be administered to a subject such as a mammal (e.g. a human, bovine, porcine, ovine, caprine, equine, cervine, canine or feline subject) in conjunction with (simultaneously or sequentially) one or more immuno-regulatory agents selected from, for example, immunoglobulins, antibiotics, interleukins (e.g. IL-2, IL-12), and/or cytokines (e.g. IFN-γ).

In yet further embodiments, the invention provides a process for the production of a one or more of polypeptides of the invention, which process comprises expressing one or more nucleic acid molecules coding for one, two or more of said polypeptides in a suitable host, and recovering the polypeptide product(s). Preferably, the host is a human cell.

There are many established algorithms available to align two amino acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid sequences for comparison may be conducted, for example, by computer-implemented algorithms (e.g. GAP BESTFIT, FASTA or TFASTA), or BLAST and BLAST 2.0 algorithms.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. (See Altschul et al. (1997) supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, score=100, wordlength=12.

One of the important parameters governing the sensitivity of BLAST searches is the word size. The most important reason that blastn is more sensitive than MEGABLAST is that it uses a shorter default word size (11). Because of this, blastn is better than MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsltr/FallWinter02/blastlab.html). This page uses an algorithm which is similar to that reported by Ma et al. (Bioinformatics. 2002 March; 18 (3): 440-5). Rather than requiring exact word matches as seeds for alignment extension, discontiguous megablast uses non-contiguous word within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

In some embodiments, the BLASTP 2.5.0+ algorithm may be used (such as that available from the NCBI) using the default parameters. In other embodiments, a BLAST Global Alignment program may be used (such as that available from the NCBI) using a Needleman-Wunsch alignment of two protein sequences with the gap costs: Existence 11 and Extension 1.

The disclosure of each reference set forth herein is specifically incorporated herein by reference in its entirety.

### BRIEF DESCRIPTION OF THE FIGURES

FIG. 1: A multi-locus representation of epitopes on a monomer of haemagglutinin (HA). Each influenza strain is assumed to contain specific epitopes of high variability as well as epitopes of low variability shared with other strains.

FIG. 2: Cyclical dynamics with strong single strain dominance generated by the antigenic thrift model based on the assumption outlined in FIG. 1.

FIG. 3A: Potential B-cell epitopes of diameter 1000A and accessibility cut-off of 10 in the head region of HA in structure 4we4, aligned in sequence space. Each column represents a residue, while each row corresponds to a potential epitope. MAIZ is picked out in yellow within this landscape; the variable residues can be read within the zoomed in box (note our numbering=standard+2).

FIG. 3B: We used available data on crystal structures of H3 haemagglutinin, along with extensive experiments using the Google CoLab alphafold platform to predict structures from known sequences, to analyse variation in the MAIZ epitope of H3. These exercises reveal that MAIZ has cycled through a set of conformations and returned to its Mu form, once again providing further empirical confirmation of the fundamental model predictions upon which this vaccine is based. The primary discriminant here is the fourth residue which flips between G and R (or occasionally K, also positively charged). The variants may be further differentiated on the basis of amino acid combinations in positions 6 and 7.

FIG. 3C: Potential B-cell epitopes of diameter 1000A and accessibility cut-off of 10 in the head region of HA in structure 4we4, aligned in sequence space. Each column represents a residue, while each row corresponds to a potential epitope. INDY is picked out in yellow within this landscape; the variable residues can be read within the zoomed in box (note our numbering=standard+2).

FIG. 3D: We used available data on crystal structures of H3 haemagglutinin, along with extensive experiments using the Google CoLab alphafold platform to predict structures from known sequences, to analyse variation in the INDY epitope of H3, shown in dark grey. These exercises confirm that INDY cycled through a set of conformations between 1968 and 2020, returned to its Alpha form in 2009. However, since 2021, a fifth conformation (Omicron) has dominated which contains a positively charged (K) residue in the 1st position. The MAIZ variant is shown in light grey, for reference.

FIG. 4A: Maximum likelihood phylogenetic tree of the haemagglutinin protein of Influenza A virus (HA3) inferred with the software IQTree. It presents the variants of epitope MAIZ (symbols) according to observed genetic variation over the decades as described in Example 4. The tree includes N=670 sequences selected randomly from all available human HA3 sequences on Genbank.

FIG. 4B: Maximum likelihood phylogenetic tree of the haemagglutinin protein of Influenza A virus (HA3) inferred with the software IQTree. It presents the variants of epitope INDY (symbols) according to observed genetic variation over the decades as described in Example 4. The tree includes N=670 sequences selected randomly from all available human HA3 sequences on Genbank.

FIG. 5: Sequential vaccination using chimeric HA constructs. Five groups of mice are sequentially vaccinated with the epitope sequences, substituted into H4, H7 and H10 HAs. Two further groups are sequentially vaccinated with H4, H7 and H10 constructs without any sequence substituted into them. A further two groups are mock vaccinated. Pseudotype microneutralisation assays using 0.5 μl of sera from the bleeds at 21 weeks are performed to detect neutralising activity. This is followed by challenges with 10³ Pfu of H3 1968 (X31) or 10⁴ Pfu H3 1978 (X79). After 3 weeks recovery, the mice undergo a terminal cardiac bleed to collect blood for analysis. Daily weight loss and percentage survival of the mice are monitored during the challenge.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Antigenic Thrift Model

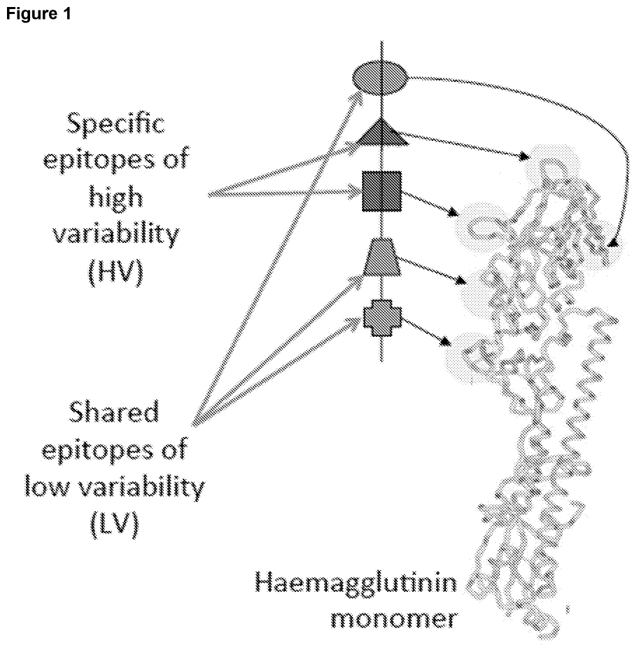

The existence of protective epitopes of low variability is consistent with the population dynamics of influenza B under the “antigenic thrift model”. This model is based on a multi-locus representation of the virus with each locus corresponding to an epitope region and presents an alternative to the more widely accepted “antigenic drift” model in having the potential to contain protective epitopes of limited variability as well as those of high variability. FIG. 1 shows how these may locate to the known antigenic sites on a monomer of haemagglutinin (HA). The epidemic behaviour of influenza can be readily explained within the antigenic thrift framework by assuming that most influenza strains are in competition with each other because they share epitopes in regions of low variability (Recker et al. 2007; Wikramaratna et al. 2013). Thus although new strains may be generated constantly through mutation, most of these cannot expand in the host population due to pre-existing immune responses against their less variable epitopes.

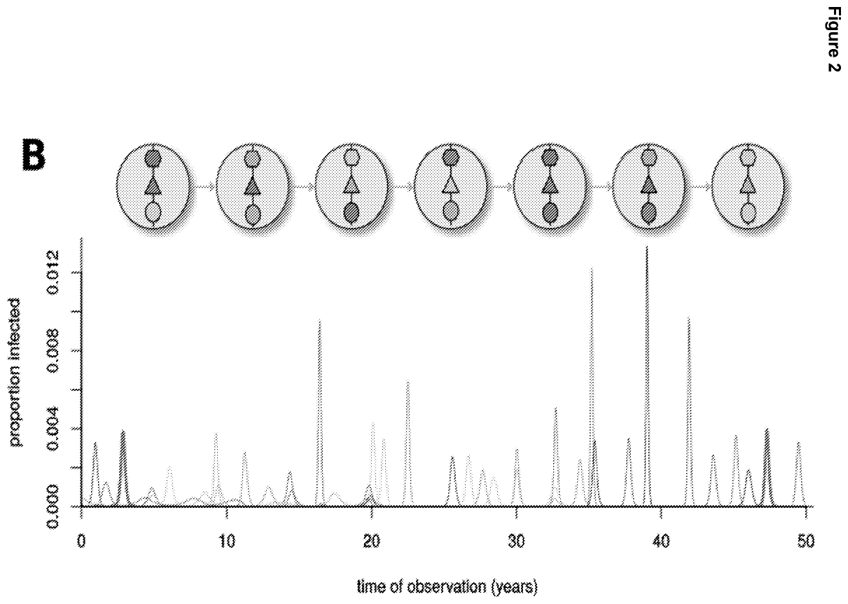

This leads to cyclical dominance of antigenic types (FIG. 2). By contrast with the “antigenic drift” model, antigenic distance between epidemic strains does not necessarily accumulate with time; instead it periodically expands and contracts.

### Example 2: Cyclical Cross-Reactivity of Infant Plasma Against Chronologically-Dispersed H3 Influenza Strains

Plasma from children aged 12 to 17 months, collected in 2009 and 2012-13, cross-reacted with HA1 domains from H3N2 influenza strains obtained in 1968 and 1972.

The fact that this plasma reacted with a panel of historical H3N2 strains in a cyclical manner leads us to infer that epitopes of limited variability are present in the head domain of H1 HA and that they cycle through a limited number of conformations as host population immunity changes.

Concordant serological studies
IC50

| | | |
|---|---|---|
| 1992 | 1972 | 1968 |

12-18 months taken in 2009

| | | |
|---|---|---|
| 0 | 20.78 | 0 |
| 0 | 10.72 | 0 |
| 0 | 4.963 | 0 |
| 0 | 74.88 | 26.66 |
| 0 | 12.86 | 24.82 |
| 0 | 18.24 | 0 |
| 0 | 21.83 | 0 |
| 0 | 42.96 | 0 |
| 0 | 81.6 | 0 |
| 0 | 28.19 | 0 |
| 0 | 18713 | 377.3 |
| 0 | 26222 | 567.3 |
| 0 | 0 | 551.6 |
| 0 | 15720 | 446.7 |
| 0 | 22700 | 434.4 |
| 0 | 8026 | 29.1 |
| 0 | 14651 | 24.34 |
| 0 | 0 | 0 |
| 0 | 9904 | 26.66 |
| 0 | 9953 | 30.63 |
| 0 | 11051 | 0 |
| 0 | 18076 | 26.03 |
| 0 | 0 | 31.21 |
| 0 | 11630 | 27.23 |
| 0 | 13581 | 33.7 |
| 0 | 1032 | 0 |
| 0 | 3385 | 0 |

12-18 months taken in 2012/2013

| | | |
|---|---|---|
| 0 | 5427 | 438.7 |
| 0 | 3089 | 617.4 |
| 0 | 1315 | 811.6 |
| 0 | 3411 | 1609 |
| 0 | 8054 | 3981 |
| 0 | 0 | 0 |
| 0 | 6157 | 525.8 |
| 0 | 4304 | 457.5 |
| 0 | 2031 | 520.9 |
| 0 | 5562 | 451.8 |
| 0 | 2851 | 483.5 |
| 0 | 4586 | 1634 |
| 0 | 2581 | 2701 |
| 0 | 5990 | 1852 |
| 0 | 11958 | 857.6 |
| 0 | 7093 | 589.3 |
| 0 | 8429 | 327.3 |
| 0 | 7479 | 648.6 |
| 0 | 6270 | 524.3 |
| 0 | 12346 | 349.7 |
| 0 | 3459 | 17169 |
| 0 | 6646 | 1427 |
| 0 | 7820 | 1261 |
| 0 | 1896 | 786.9 |
| 0 | 5298 | 849 |
| 0 | 3459 | 810.8 |
| 0 | 4413 | 534.2 |
| 0 | 2414 | 879.1 |
| 0 | 1201 | 919.9 |
| 0 | 3788 | 488.5 |

These data show cross-reactivity between strains circulating in periods when the alpha variant of INDY was dominant.

### Example 3: Identification of Epitopes of Limited Variability

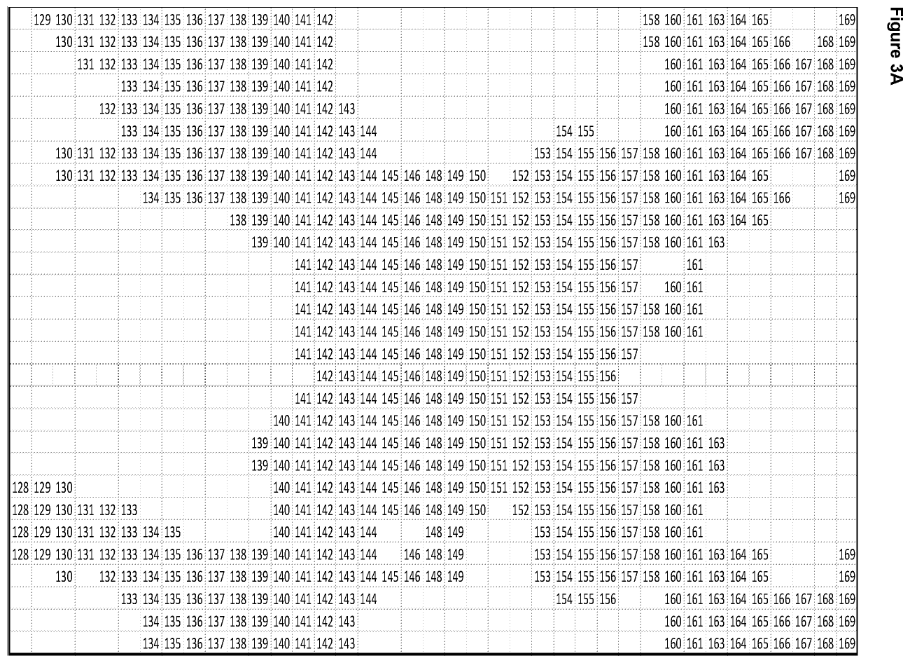

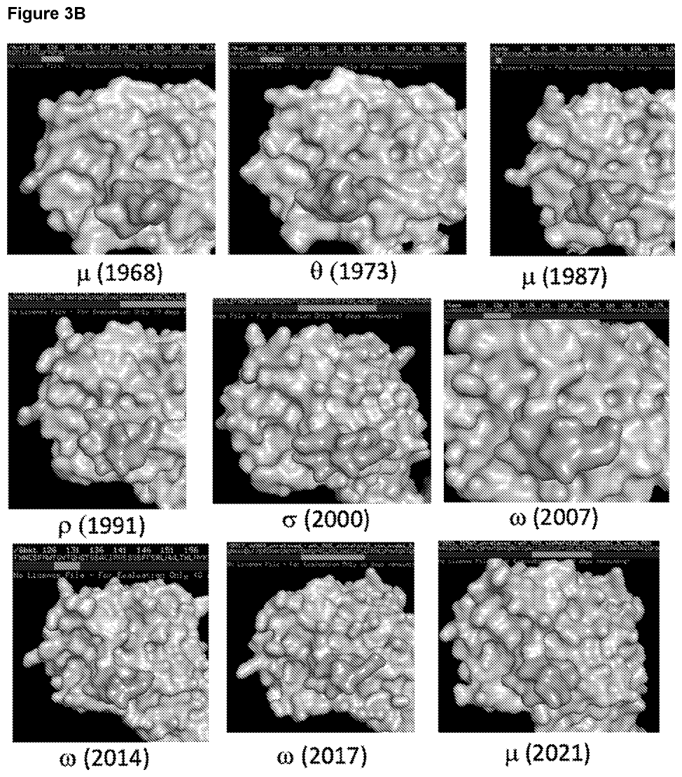

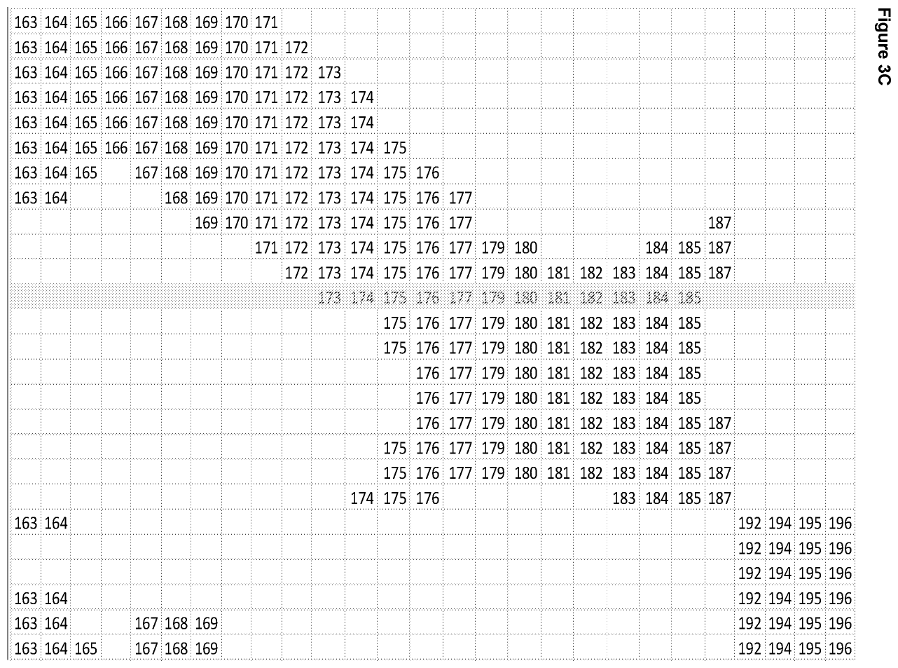

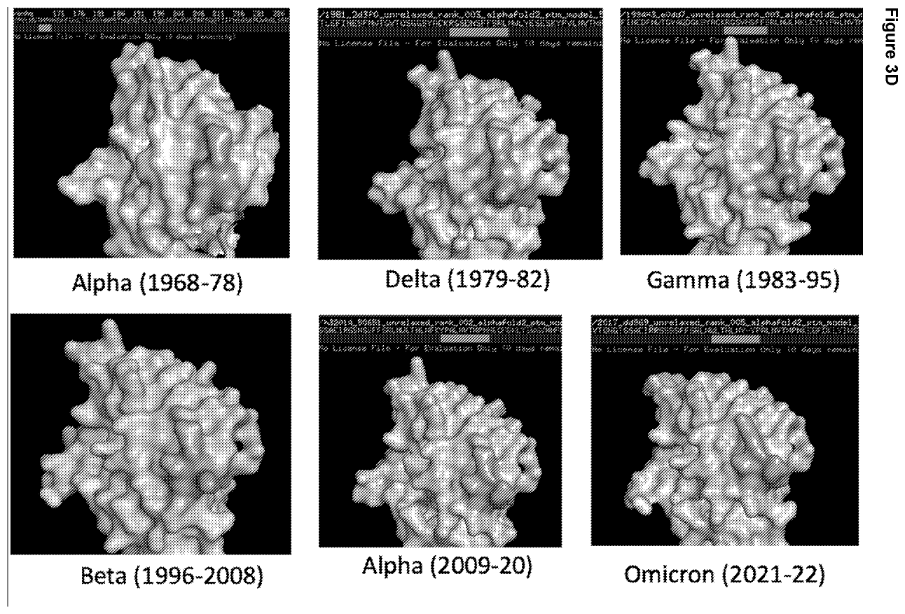

To identify epitopes of limited variability, the following steps were performed:
1. Genetic sequences of existing protein structures (EPS) were aligned to thousands of available H3 genetic sequences in Genbank to match residues, allowing us to quantify historical genetic diversity per residue
2. Accessibility of residues on the surface of the protein was calculated under various thresholds (e.g. 1%, 10%, 30%) using standard tools like DSSP (https://swift.cmbi.umcn.nl/gv/dssp/) and FreeSASA (https://freesasa.github.io/), allowing us to determine if a residue is accessible/visible to an antibody targeting the surface region that includes that residue
3. For each accessible residue, various binding site areas (e.g. 600 A², 800 A² or 1000 A²) were considered for an antibody targeting a region centred on such residue, allowing us to determine what other accessible residues would that potential antibody be able to access
4. Steps 1-3 resulted in a list of potential antibodies centred at each of the accessible residues of each EPS, allowing us to determine which antibodies would be universal (UAB) in the sense that they would potentially access the same range of residues across all EPS (FIGS. 3A-3K).
5. Considering the UABs and the involved residues, we then mined historical genetic data of thousands of available H3 genetic sequences in Genbank, verifying if their genetic signatures cycled/varied in time, allowing us to subset all possible UABs to those that match theoretical expectations from the theory of influenza genetic thrift
6. The epitopes were categorised by properties of the key variable amino acids with respect to charge and size.

### Example 4: Cycling of Epitopes

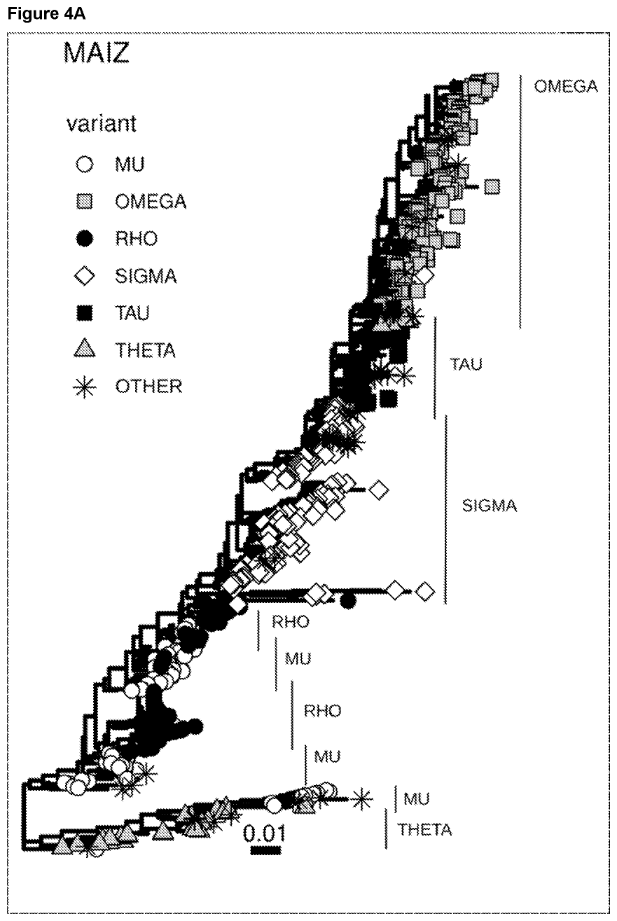

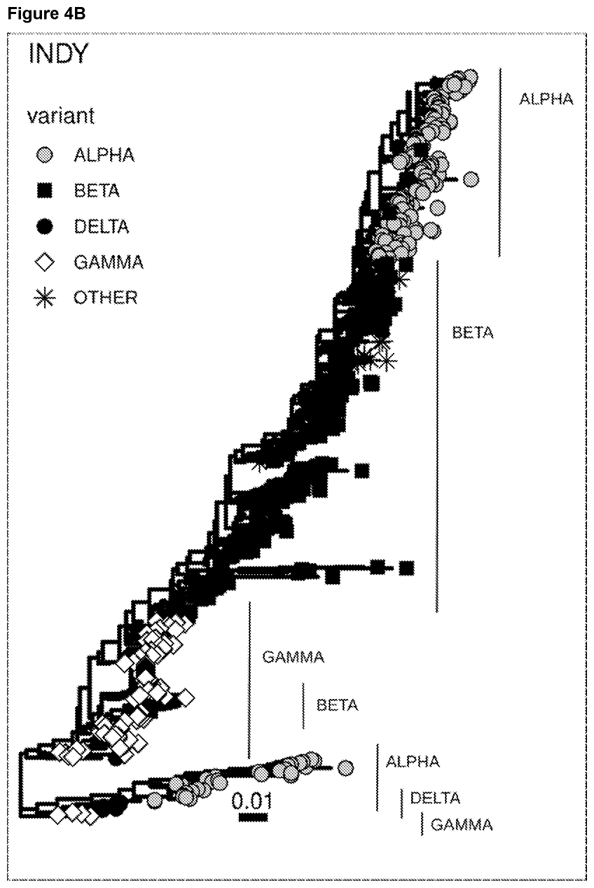

The cycling of epitopes is evident upon the inspection of the tables below showing the years in which each was in circulation. FIGS. 4A and 4B locate these to the phylogenetic tree of H3N2, underscoring that these epitopes have re-emerged at different points in time.

Epitope 1 (“MAIZ”)

| STANDARD | | | | | | | | | | | | | | |
|---|---|---|---|---|---|---|---|---|---|---|---|---|---|---|
| 141 | 142 | 143 | 144 | 145 | 146 | 147 | 148 | 149 | 150 | 151 | 152 | 153 | 154 | 155 |
| LINEAR | | | | | | | | | | | | | | |
| 155 | 156 | 157 | 158 | 159 | 160 | 161 | 162 | 163 | 164 | 165 | 166 | 167 | 168 | 169 |

OMEGA

| | | | | | | | | | | | | | | | |
|---|---|---|---|---|---|---|---|---|---|---|---|---|---|---|---|
| >2017 | C | I | R | R | S | S | S | S | F | F | S | R | L | N | W |
| >2016 | C | I | R | R | S | S | S | S | F | F | S | R | L | N | W |
| >2015 | C | I | R | R | S | S | S | S | F | F | S | R | L | N | W |
| >2014 | C | I | R | R | S | S | S | S | F | F | S | R | L | N | W |
| >2013 | C | I | R | G | S | N | S | S | F | F | S | R | L | N | W |
| >2012 | C | I | R | R | S | N | S | S | F | F | S | R | L | N | W |
| >2011 | C | I | R | R | S | N | N | S | F | F | S | R | L | N | W |
| >2010 | C | I | R | R | S | N | N | S | F | F | S | R | L | N | W |
| >2009 | C | I | R | R | S | K | N | S | F | F | S | R | L | N | W |
| >2008 | C | I | R | R | S | N | N | S | F | F | S | R | L | N | W |
| >2007 | C | I | R | R | S | N | N | S | F | F | S | R | L | N | W |

TAU

| | | | | | | | | | | | | | | | |
|---|---|---|---|---|---|---|---|---|---|---|---|---|---|---|---|
| >2006 | C | K | R | R | S | N | N | S | F | F | S | R | L | N | W |
| >2005 | C | K | R | R | S | N | N | S | F | F | S | R | L | N | W |
| >2004 | C | K | R | R | S | N | N | S | F | F | S | R | L | N | W |

SIGMA

| | | | | | | | | | | | | | | | |
|---|---|---|---|---|---|---|---|---|---|---|---|---|---|---|---|
| >2003 | C | K | R | R | S | N | K | S | F | F | S | R | L | N | W |
| >2002 | C | K | R | R | S | D | K | S | F | F | S | R | L | N | W |
| >2001 | C | K | R | R | S | N | K | S | F | F | S | R | L | N | W |
| >2000 | C | K | R | R | S | N | K | S | F | F | S | R | L | N | W |
| >1999 | C | K | R | R | S | I | K | S | F | F | S | R | L | N | W |
| >1998 | C | K | R | R | S | I | K | S | F | F | S | R | L | N | W |
| >1997 | C | K | R | R | S | V | K | S | F | F | S | R | L | N | W |

RHO

| | | | | | | | | | | | | | | | |
|---|---|---|---|---|---|---|---|---|---|---|---|---|---|---|---|
| >1996 | C | K | R | G | S | V | K | S | F | F | S | R | L | N | W |
| >1992 | C | K | R | G | S | V | K | S | F | F | S | R | L | N | W |
| >1991 | C | K | R | G | S | V | K | S | F | F | S | R | L | N | W |

MU

| | | | | | | | | | | | | | | | |
|---|---|---|---|---|---|---|---|---|---|---|---|---|---|---|---|
| >1995 | C | K | R | G | S | V | N | S | F | F | S | R | L | N | W |
| >1994 | C | K | R | G | S | V | N | S | F | F | S | R | L | N | W |
| >1993 | C | K | R | G | S | V | N | S | F | F | S | R | L | N | W |
| >1990 | C | K | R | G | S | V | N | S | F | F | S | R | L | N | W |
| >1989 | C | K | R | G | S | V | N | S | F | F | S | R | L | N | W |
| >1988 | C | K | R | G | S | V | N | S | F | F | S | R | L | N | W |
| >1987 | C | K | R | G | S | V | N | S | F | F | S | R | L | N | W |
| >1986 | C | K | R | G | S | V | N | S | F | F | S | R | L | N | W |
| >1985 | C | K | R | G | S | V | N | S | F | F | S | R | L | N | W |
| >1984 | C | K | R | G | S | V | N | S | F | F | S | R | L | N | W |
| >1983 | C | K | R | G | S | V | N | S | F | F | S | R | L | N | W |
| >1971 | C | K | R | G | P | G | S | G | F | F | S | R | L | N | W |
| >1970 | C | K | R | G | P | G | S | G | F | F | S | R | L | N | W |
| >1969 | C | K | R | G | P | G | S | G | F | F | S | R | L | N | W |
| >1968 | C | K | R | G | P | G | S | G | F | F | S | R | L | N | W |

THETA

| | | | | | | | | | | | | | | | |
|---|---|---|---|---|---|---|---|---|---|---|---|---|---|---|---|
| >1982 | C | K | R | G | S | D | N | S | F | F | S | R | L | N | W |
| >1981 | C | K | R | G | S | D | N | S | F | F | S | R | L | N | W |
| >1980 | C | K | R | G | S | D | N | S | F | F | S | R | L | N | W |
| >1979 | C | K | R | G | S | D | K | S | F | F | S | R | L | N | W |
| >1978 | C | K | R | G | P | D | N | S | F | F | S | R | L | N | W |
| >1977 | C | K | R | G | P | D | N | S | F | F | S | R | L | N | W |
| >1976 | C | K | R | G | P | D | N | G | F | F | S | R | L | N | W |
| >1975 | C | K | R | G | P | D | S | G | F | F | S | R | L | N | W |
| >1974 | C | K | R | G | P | D | S | G | F | F | S | R | L | N | W |
| >1973 | C | K | R | G | P | D | S | G | F | F | S | R | L | N | W |
| >1972 | C | K | R | G | P | D | S | G | F | F | S | R | L | N | W |

Epitope 2 (“INDY”)

| | STANDARD | | | | | | | | | | | | | |
|---|---|---|---|---|---|---|---|---|---|---|---|---|---|---|
| | 128 | 129 | 130 | 131 | 132 | 173 | 174 | 175 | 176 | 177 | 178 | 179 | 180 | 181 |
| | LINEAR | | | | | | | | | | | | | |
| | 142 | 143 | 144 | 145 | 146 | 187 | 188 | 189 | 190 | 191 | 192 | 193 | 194 | 195 |
| >2017 | N | W | T | G | V | K | E | Q | F | D | K | L | Y | I |
| >2016 | N | W | T | G | V | N | E | Q | F | D | K | L | Y | I |
| >2015 | N | W | T | G | V | N | E | Q | F | D | K | L | Y | I |
| >2014 | N | W | T | G | V | N | E | Q | F | D | K | L | Y | I |
| >2013 | N | W | A | G | V | N | E | Q | F | D | K | L | Y | I |
| >2012 | N | W | T | G | V | N | E | Q | F | D | K | L | Y | I |
| >2011 | N | W | T | G | V | N | E | Q | F | D | K | L | Y | I |
| >2010 | N | W | T | G | V | N | E | Q | F | D | K | L | Y | I |
| >2009 | N | W | T | G | V | N | E | Q | F | D | K | L | Y | I |
| >2008 | N | W | T | G | V | N | E | Q | F | D | K | L | Y | I |
| >1978 | N | W | T | G | V | N | D | N | F | D | K | L | Y | I |
| >1977 | N | W | T | G | V | N | D | N | F | D | K | L | Y | I |
| >1976 | N | W | T | G | V | N | D | N | S | D | K | L | Y | I |
| >1975 | N | W | T | G | V | N | D | N | F | D | K | L | Y | I |
| >1974 | N | W | T | G | V | N | D | N | F | D | K | L | Y | I |
| >1973 | T | W | T | G | V | N | D | N | F | D | K | L | Y | I |
| >1972 | T | W | T | G | V | N | D | N | F | D | K | L | Y | I |
| >1971 | T | W | T | G | V | N | D | N | F | D | K | L | Y | I |
| >1970 | T | W | T | G | V | N | D | N | F | D | K | L | Y | I |
| >1969 | T | W | T | G | V | N | D | N | F | D | K | L | Y | I |
| >1968 | T | W | T | G | V | N | D | N | F | D | K | L | Y | I |

BETA

| | | | | | | | | | | | | | | |
|---|---|---|---|---|---|---|---|---|---|---|---|---|---|---|
| >2007 | N | W | T | G | V | N | E | K | F | D | K | L | Y | I |
| >2006 | N | W | T | G | V | N | E | K | F | D | K | L | Y | I |
| >2005 | N | W | T | G | V | N | E | K | F | D | K | L | Y | I |
| >2004 | N | W | T | G | V | N | E | K | F | D | K | L | Y | I |
| >2003 | D | W | T | G | V | N | E | K | F | D | K | L | Y | I |
| >2002 | N | W | T | G | V | N | E | K | F | D | K | L | Y | I |
| >2001 | N | W | T | G | V | N | E | K | F | D | K | L | Y | I |
| >2000 | N | W | T | G | V | N | E | K | F | D | K | L | Y | I |
| >1999 | N | W | T | G | V | N | E | K | F | D | K | L | Y | I |
| >1998 | N | W | T | G | V | N | D | K | F | D | K | L | Y | I |
| >1997 | N | W | T | G | V | N | D | K | F | D | K | L | Y | I |
| >1996 | N | W | T | G | V | N | D | K | F | D | K | L | Y | I |
| >1995 | N | W | T | G | V | N | D | K | F | D | K | L | Y | I |
| >1994 | N | W | T | G | V | N | G | K | F | D | K | L | Y | I |
| >1993 | N | W | T | G | V | N | G | K | F | D | K | L | Y | I |
| >1992 | N | W | T | G | V | N | G | K | F | D | K | L | Y | I |
| >1991 | N | W | T | G | V | N | G | K | F | D | K | L | Y | I |
| >1990 | N | W | T | G | V | N | G | K | F | D | K | L | Y | I |
| >1989 | N | W | T | G | V | N | G | K | F | D | K | L | Y | I |
| >1988 | N | W | T | G | V | N | G | K | F | D | K | L | Y | I |
| >1987 | N | W | T | G | V | N | G | K | F | D | K | L | Y | I |
| >1986 | N | W | T | G | V | N | G | K | F | D | K | L | Y | I |
| >1985 | N | W | T | G | V | N | G | K | F | D | K | L | Y | I |
| >1984 | N | W | T | G | V | N | G | K | F | D | K | L | Y | I |
| >1983 | N | W | T | G | V | N | G | K | F | D | K | L | Y | I |
| >1982 | N | W | T | G | V | N | G | K | F | D | K | L | Y | I |

DELTA

| >1981 | N | W | T | G | V | N | G | N | F | D | K | L | Y | I |
|---|---|---|---|---|---|---|---|---|---|---|---|---|---|---|
| >1980 | N | W | T | G | V | N | G | N | F | D | K | L | Y | I |
| >1979 | N | W | T | G | V | N | G | N | F | D | K | L | Y | I |
| | STANDARD | | | | | | | | | | | | | |
| | 182 | 183 | 184 | 213 | 214 | 215 | 242 | 243 | 245 | 246 | 247 | 248 | 249 | 251 |
| | LINEAR | | | | | | | | | | | | | |
| | 196 | 197 | 198 | 227 | 228 | 229 | 256 | 257 | 259 | 260 | 261 | 262 | 263 | 265 |
| >2017 | W | G | V | Q | A | V | G | D | L | L | I | N | S | G |
| >2016 | W | G | V | Q | A | V | G | D | L | L | I | N | S | G |
| >2015 | W | G | V | Q | A | V | G | D | L | L | I | N | S | G |
| >2014 | W | G | V | Q | A | V | G | D | L | L | I | N | S | G |
| >2013 | W | G | V | Q | A | V | G | D | L | L | I | N | S | G |
| >2012 | W | G | V | Q | A | V | G | D | L | L | I | N | S | G |
| >2011 | W | G | V | Q | A | V | G | D | L | L | I | N | S | G |
| >2010 | W | G | V | Q | A | V | G | D | L | L | I | N | S | G |
| >2009 | W | G | V | Q | T | V | G | D | L | L | I | N | S | G |
| >2008 | W | G | V | Q | T | V | G | D | L | L | I | N | S | G |
| >1978 | W | G | V | Q | T | V | G | D | L | L | I | N | S | G |
| >1977 | W | G | V | Q | T | V | G | D | L | L | I | N | S | G |
| >1976 | W | G | V | Q | T | V | G | D | L | V | I | N | S | G |
| >1975 | W | G | V | Q | T | I | G | D | L | V | I | N | S | G |
| >1974 | W | G | V | Q | T | I | G | D | L | V | I | N | S | G |
| >1973 | W | G | V | Q | T | I | G | D | L | V | I | N | S | G |
| >1972 | W | G | V | Q | T | I | G | D | L | V | I | N | S | G |
| >1971 | W | G | V | Q | T | I | G | D | L | V | I | N | S | G |
| >1970 | W | G | V | Q | T | I | G | D | L | V | I | N | S | G |
| >1969 | W | G | V | Q | T | I | G | D | L | V | I | N | S | G |
| >1968 | W | G | V | Q | T | I | G | D | L | V | I | N | S | G |
| | BETA | | | | | | | | | | | | | |
| >2007 | W | G | V | Q | T | V | G | D | L | L | I | N | S | G |
| >2006 | W | G | V | Q | T | V | G | D | L | L | I | N | S | G |
| >2005 | W | G | V | Q | T | V | G | D | L | L | I | N | S | G |
| >2004 | W | G | V | Q | T | V | G | D | L | L | I | N | S | G |
| >2003 | W | G | V | Q | T | V | G | D | L | L | I | N | S | G |
| >2002 | W | G | V | Q | T | V | G | D | L | L | I | N | S | G |
| >2001 | W | G | V | Q | T | V | G | D | L | L | I | N | S | G |
| >2000 | W | G | V | Q | T | V | G | D | L | L | I | N | S | G |
| >1999 | W | G | V | Q | T | V | G | D | L | L | I | N | S | G |
| >1998 | W | G | V | Q | T | V | G | D | L | L | I | N | S | G |
| >1997 | W | G | V | Q | T | V | G | D | L | L | I | N | S | G |
| >1996 | W | G | V | Q | T | V | G | D | L | L | I | N | S | G |
| >1995 | W | G | V | Q | T | V | G | D | L | L | I | N | S | G |
| >1994 | W | G | V | Q | T | V | G | D | L | L | I | N | S | G |
| >1993 | W | G | V | Q | T | V | G | D | L | L | I | N | S | G |
| >1992 | W | G | V | Q | T | V | G | D | L | L | I | N | S | G |
| >1991 | W | G | V | Q | T | V | G | D | L | L | I | N | S | G |
| >1990 | W | G | V | Q | T | V | G | D | L | L | I | N | S | G |
| >1989 | W | G | V | Q | T | V | G | D | L | L | I | N | S | G |
| >1988 | W | G | V | Q | T | V | G | D | L | L | I | N | S | G |
| >1987 | W | G | V | Q | T | V | G | D | L | L | I | N | S | G |
| >1986 | W | G | V | Q | T | V | G | D | L | L | I | N | S | G |
| >1985 | W | G | V | Q | T | V | G | D | L | L | I | N | S | G |
| >1984 | W | G | V | Q | T | V | G | D | L | L | I | N | S | G |
| >1983 | W | G | V | Q | T | V | G | D | L | L | I | N | S | G |
| >1982 | W | G | V | Q | T | V | G | D | L | L | I | N | S | G |
| | DELTA | | | | | | | | | | | | | |
| >1981 | W | G | V | Q | T | I | G | D | L | L | I | N | S | G |
| >1980 | W | G | V | Q | T | I | G | D | L | L | I | N | S | G |
| >1979 | W | G | V | Q | T | I | G | D | L | L | I | N | S | G |

OMICRON

| | STANDARD | | | | | | | | | | | |
|---|---|---|---|---|---|---|---|---|---|---|---|---|
| | 171 | 172 | 173 | 174 | 175 | 176 | 177 | 178 | 179 | 180 | 181 | 182 |
| | LINEAR | | | | | | | | | | | |
| | 187 | 188 | 189 | 190 | 191 | 192 | 193 | 194 | 195 | 196 | 197 | 198 |
| 2022 | K | E | Q | F | D | K | L | Y | I | W | G | V |
| 2021 | K | E | Q | F | D | K | L | Y | I | W | G | V |

### Example 5: Synthesis of Polypeptides

Invitrogen® GeneArt Strings are used to synthesise the chimeric HA molecules consisting of the epitope of limited variability substituted into the HA1 domain of the Victoria and Yamagata strains. Chimeric influenza A H4 head domains comprising the following epitopes are synthesised:

Epitope 1 (“MAIZ”)

| | STANDARD | | | | | | | | | | | | | | |
|---|---|---|---|---|---|---|---|---|---|---|---|---|---|---|---|
| | 141 | 142 | 143 | 144 | 145 | 146 | 147 | 148 | 149 | 150 | 151 | 152 | 153 | 154 | 155 |
| | LINEAR | | | | | | | | | | | | | | |
| | 155 | 156 | 157 | 158 | 159 | 160 | 161 | 162 | 163 | 164 | 165 | 166 | 167 | 168 | 169 |
| OMEGA | C | I | R | R | S | S | S | S | F | F | S | R | L | N | W |
| TAU | C | K | R | R | S | N | N | S | F | F | S | R | L | N | W |
| SIGMA | C | K | R | R | S | N | K | S | F | F | S | R | L | N | W |
| RHO | C | K | R | G | S | V | K | S | F | F | S | R | L | N | W |
| MU | C | K | R | G | S | V | N | S | F | F | S | R | L | N | W |
| THETA | C | K | R | G | S | D | N | S | F | F | S | R | L | N | W |

Epitope 2 (“INDY”)

| | STANDARD | | | | | | | | | | | | | |
|---|---|---|---|---|---|---|---|---|---|---|---|---|---|---|
| | 128 | 129 | 130 | 131 | 132 | 173 | 174 | 175 | 176 | 177 | 178 | 179 | 180 | 181 |
| | LINEAR | | | | | | | | | | | | | |
| | 142 | 143 | 144 | 145 | 146 | 187 | 188 | 189 | 190 | 191 | 192 | 193 | 194 | 195 |
| ALPHA | N | W | T | G | V | K | E | Q | F | D | K | L | Y | I |
| BETA | N | W | T | G | V | N | E | K | F | D | K | L | Y | I |
| GAMMA | N | W | T | G | V | N | G | K | F | D | K | L | Y | I |
| DELTA | N | W | T | G | V | N | G | N | F | D | K | L | Y | I |
| | STANDARD | | | | | | | | | | | | | |
| | 182 | 183 | 184 | 213 | 214 | 215 | 242 | 243 | 245 | 246 | 247 | 248 | 249 | 251 |
| | LINEAR | | | | | | | | | | | | | |
| | 196 | 197 | 198 | 227 | 228 | 229 | 256 | 257 | 259 | 260 | 261 | 262 | 263 | 265 |
| ALPHA | W | G | V | Q | A | V | G | D | L | L | I | N | S | G |
| BETA | W | G | V | Q | T | V | G | D | L | L | I | N | S | G |
| GAMMA | W | G | V | Q | T | V | G | D | L | L | I | N | S | G |
| DELTA | W | G | V | Q | T | I | G | D | L | L | I | N | S | G |

The Omicron sequence (KEQFDKLYIWGV) is also made.

The chimeric HA1 domain sequences are then cloned into DNA expression constructs and lentiviral glycoprotein expression vectors. The DNA expression vectors are grown up in E. coli and purified using a Qiagen Giga Prep Kit. Lentiviruses are produced displaying the chimeric HAs via the protocol outlined in Carnell et al. (2015) before being purified by sucrose cushion centrifugation.

### Example 6: Mouse Challenges

Mouse Influenza Challenges are Performed with Influenza Strains:

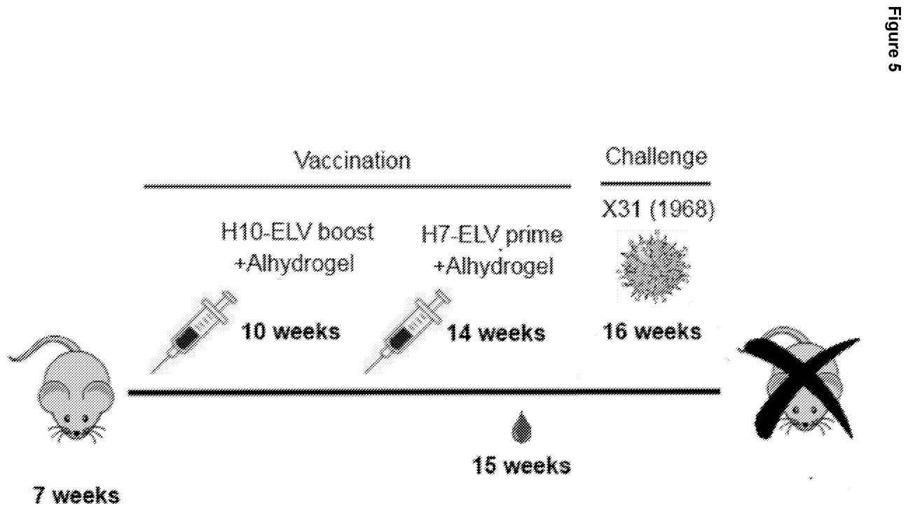

Five groups of mice are sequentially vaccinated with the sequences outlined above, substituted into H4, H7 and H10 HAs. Two further groups are sequentially vaccinated with H4, H7 and H10 constructs without any sequence substituted into them. A further two groups are mock vaccinated. Pseudotype micro-neutralisation assays using 0.5 μl of sera from the bleeds at 21 weeks are performed to detect neutralising activity. This is followed by challenge with 10³ Pfu of H3 1968 (X31) or 10⁴ Pfu H3 1978 (X79). After 3 weeks recovery, the mice undergo a terminal cardiac bleed to collect blood for analysis. Daily weight loss and percentage survival of the mice are monitored during the challenge. The basic vaccination protocol is shown in FIG. 5.

### REFERENCES

Belongia, E. A. et al., 2009. Effectiveness of Inactivated Influenza Vaccines Varied Substantially with Antigenic Match from the 2004-2005 Season to the 2006-2007 Season Linked references are available on JSTOR for this article: Effectiveness of Inactivated Influenza Vaccines Varied. The Journal of Infectious Disease, 199 (2), pp. 159-167.
Carnell et al., (2015) Pseudotype-based neutralization assays for influenza: a systematic analysis. Front Immunol. 2015 Apr. 29; 6:161. doi: 10.3389/fimmu.2015.00161. eCollection 2015.
Carter et al., (2013) Sequential seasonal H1N1 influenza virus infections protect ferrets against novel 2009 H1N1 influenza virus. J Virol. 2013 Feb.;87 (3): 1400-10.
Caton et al., 1982. The antigenic structure of the influenza virus A/PR/8/34 hemagglutinin (H1 subtype). Cell, 31 (2 Pt 1), pp. 417-427.
Gupta S. 2016 Immune Driven Pathogen Evolution, Encyclopaedia of Immunology (Ed. Kaye, P.) Elsevier.
Krammer, F. et al., 2013. Broadly Protective Stalk-Specific Antibodies., 87 (12), pp. 6542-6550.
Li, Y. et al., 2013. Immune history shapes specificity of pandemic H1N1 influenza antibody responses. 210 (8), pp. 1493-1500.
Lozano, R. et al., 2012. Global and regional mortality from 235 causes of death for 20 age groups in 1990 and 2010: a systematic analysis for the Global Burden of Disease Study 2010. Lancet, 380, pp. 2095-2128.
Manicassamy, B. et al., 2010. Protection of mice against lethal challenge with 2009 H1N1 influenza A virus by 1918-like and classical swine H1N1 based vaccines. PLOS Pathogens, 6 (1).
Matsuzaki, Y. et al., 2014. Epitope Mapping of the Hemagglutinin Molecule of A/(H1N1) pdm09 Influenza Virus by Using Monoclonal Antibody Escape Mutants. Journal of Virology, 88 (21), pp. 12364-12373.
Mertz, D., Hyong, T. & Johnstone, J., 2013. Populations at risk for severe or complicated influenza illness: systematic review and meta-analysis. British Medical Journal, 5061 (August), pp. 1-15.
Miura et al. 2008 Vaccine 26:193.
Presanis, A. M. et al., 2011. Changes in severity of 2009 pandemic A/H1N1 influenza in England: a Bayesian evidence synthesis. British Medical Journal, (343), pp. 1-14.
Recker, M. et al., 2007. The generation of influenza outbreaks by a network of host immune responses against a limited set of antigenic types. PNAS 104:7711
Taubenberger, J. K. & Morens, D. M., 2006. 1918 Influenza: the Mother of All Pandemics. Lancet, 12 (1), pp. 15-22.
Treanor, J. J. et al., 2012. Effectiveness of Seasonal Influenza Vaccines in the United States During a Season With Circulation of All Three Vaccine Strains., pp. 1-9. WHO 2016. Recommended composition of influenza virus vaccines for use in the 2016-2017 northern hemisphere influenza season.
Wikramaratna, P. S. et al., 2013. The antigenic evolution of influenza: drift or thrift? Philosophical transactions of the Royal Society of London. Series B, Biological sciences, 368 (1614), p.20120200. Available at: http://www.pubmedcentral.nih.gov/articlerender.fcgi?artid=3678325&tool=pmcentrez&re ndertype=abstract.

H3 SEQUENCES

| | |
|---|---|
| SEQ | |
| ID | |
| NO: | Sequence |

Head domain sequences

| | |
|---|---|
| 1 | Influenza A subtype H4 haemagglutinin head domain, amino acid |
| | CPSPLRLIDGQACDIINGALGSPGCDHLNGAEWDVFIERPNAVDTCYPFDIPDYQSLR |
| | SILANNGKFEFIAEEFSWTTVKQNGKSGACKRANVNDFFNRLNWLTKSDGNAYPLQN |
| | LTKVNNGDYARLYIWGVHHPSTDAEQTNLYKNNPGRVTVSTKTDQTSVVPNIGSRPW |
| | VRGQSGRISFYWTIVEPGDLIVFNTIGNLIAPRGHYKLNSQKKSTILNTAIPIGSC |
| 2 | Influenza A subtype H7 haemagglutinin head domain, amino acid |
| | CTQGKRSTDLGQCGLLGTLIGPPQCDQFLEFNADLIIERREGTDVCYPGKFTNEESLR |
| | QILRGSGGIDKESMGFTYSGIRTNGATSACRRSGSSFYAEMKWLLSNSDNAAFPQMT |
| | KSYRNPRNKPALIIWGVHHSGSTTEQTKLYGSGNKLITVGSSKYQQSFTPSPGARPQ |
| | VNGQSGRIDFHWLLLDPNDTVTFTFNGAFIAPDRASFFRGESLGVQSDVPLDSGCEG |
| | DC |
| 3 | Influenza A subtype H10 haemagglutinin head domain, amino acid |
| | CMKSRNYKDLGNCHPIGMIIGTPACDLHLTGTWDTLIERNNSIAYCYPGATVNEEALR |
| | QKIMESGGIDKISTGFTYGSSINSAGTTAACMRNGGNSFYAELKWLVSKSKGQNFPQ |
| | TTNTYRNTDSAEHLIIWGIHHPSSTQEKNDLYGTQSLSISVGSSTYQNNFVPVVGARP |
| | QVNGQSGRIDFHWTMVQPGDNITFSHNGGLIAPSRVSKLKGRGLGIQSGASVDNDC |
| 4 | Influenza A subtype H14 haemagglutinin head domain, amino acid |
| | CRLKNXLRQTTLMNYAQVLXSLSMDKTATLSTVHWEAQAVTTYKTLLGMFSLKGQRQ |
| | WTRAIHSMFQITRALEASWQAVGVWNSLPNNSLGMVSQLTDQAVLVXGAAAMASSP |
| | DXTGXPEXKTGTMGLLMSQKKIQGLTSGSISGECTIHQVILSKRIFTKLQREEXRCLLA |
| | QIKSALFPIXEVDQGXGIRAAEXASTGLXXTQGIPSSLTALETXLHQEATTKXINLQRAL |
| | C |
| 5 | Influenza A subtype H15 haemagglutinin head domain, amino acid |
| | CTKGKKAVDLGSCGILGTIIGPPQCDLHLEFKADLIIERRNSSDICYPGRFTNEEALRQII |
| | RESGGIDKESMGFRYSGIRTDGATSACKRTVSSFYSEMKWLSSSMNNQVFPQLNQT |
| | YRNTRKEPALIVWGVHHSSSLDEQNKLYGTGNKLITVGSSKYQQSFSPSPGARPKVN |
| | GQAGRIDFHWMLLDPGDTVTFTFNGAFIAPDRATFLRSNAPSGIEYNGKSLGIQSDAQ |
| | IDESC |
| 6 | Influenza A subtype H4 haemagglutinin head domain, nucleotide |
| | TGCCCGAGTCCTCTGAGATTGATTGACGGCCAGGCTTGCGACATCATAAATGGGG |
| | CATTGGGAAGCCCAGGGTGTGACCATCTGAATGGTGCAGAATGGGACGTGTTCA |
| | TAGAAAGGCCCAATGCAGTGGACACCTGTTACCCCTTCGATATTCCAGACTATCA |
| | GAGTTTGAGAAGCATACTTGCTAACAATGGGAAGTTTGAATTCATTGCTGAAGAAT |
| | TTTCATGGACCACTGTAAAACAAAATGGAAAGTCTGGAGCCTGCAAAAGGGCGAA |
| | TGTGAATGATTTCTTTAATAGGCTGAATTGGCTGACAAAGTCAGACGGAAATGCCT |
| | ATCCTCTTCAAAATTTGACGAAAGTAAATAATGGTGATTACGCAAGACTTTACATCT |
| | GGGGAGTTCATCACCCTTCAACAGACGCAGAGCAAACCAACCTATACAAGAATAA |
| | CCCTGGGAGAGTCACTGTGTCTACCAAAACTGACCAAACAAGTGTAGTCCCTAAT |
| | ATTGGCAGTAGGCCCTGGGTAAGAGGGCAAAGCGGGAGAATAAGCTTCTACTGG |
| | ACCATTGTGGAGCCCGGGGACCTGATAGTCTTCAACACAATAGGAAACCTAATTG |
| | CCCCAAGAGGGCATTACAAACTGAATAGCCAGAAAAAAAGCACAATTCTAAATACT |
| | GCAATACCAATAGGCTCATGT |
| 7 | Influenza A subtype H7 haemagglutinin head domain, nucleotide |
| | TGCACTCAGGGGAAAAGATCAACAGATCTGGGACAATGCGGACTTCTAGGAACCC |
| | TAATAGGACCTCCCCAATGCGATCAATTTTTGGAGTTTAACGCTGATTTAATAATTG |
| | AACGAAGAGAAGGAACCGATGTGTGTTATCCCGGGAAATTCACAAATGAAGAATC |
| | ACTGAGGCAGATCCTTCGAGGATCAGGAGGAATTGATAAGGAGTCAATGGGCTTC |
| | ACCTATAGTGGAATAAGAACCAATGGAGCGACGAGTGCTTGCAGAAGATCAGGTT |
| | CTTCCTTCTATGCGGAGATGAAGTGGTTGCTGTCGAATTCAGACAATGCGGCTTTT |
| | CCCCAAATGACCAAATCGTATAGAAATCCCAGGAACAAACCAGCTCTAATAATTTG |
| | GGGAGTGCATCACTCTGGATCGACTACTGAGCAAACTAAACTCTATGGGAGTGGA |
| | AACAAGTTGATAACAGTGGGAAGCTCGAAATACCAGCAGTCATTCACCCCAAGCC |
| | CGGGGGCACGACCACAAGTGAATGGGCAATCAGGAAGGATTGATTTTCATTGGCT |
| | ACTCCTTGATCCCAATGACACAGTGACTTTCACTTTCAATGGGGCATTCATAGCTC |
| | CTGACAGAGCAAGTTTCTTTAGAGGAGAATCACTAGGAGTTCAGAGTGATGTTCC |
| | TTTGGATTCTGGTTGTGAGGGGGATTGC |
| | Influenza A subtype H10 haemagglutinin head domain, nucleotide |
| | TGCATGAAAAGTCGGAATTACAAGGACTTAGGTAATTGTCACCCAATAGGGATGAT |
| | AATAGGGACTCCTGCTTGTGATCTACACCTCACCGGAACATGGGACACTTTGATA |
| | GAGAGAAACAATTCTATTGCCTACTGTTACCCGGGTGCCACTGTGAATGAAGAAG |
| | CATTAAGGCAGAAAATTATGGAAAGTGGAGGGATTGACAAGATAAGCACCGGGTT |
| | TACATATGGATCATCTATCAATTCGGCTGGAACCACTGCAGCATGCATGAGAAATG |
| | GAGGAAATAGTTTCTATGCGGAGCTAAAGTGGCTAGTATCTAAGAGCAAAGGACA |
| | AAACTTCCCACAAACAACAAACACATACAGAAATACAGATTCAGCAGAACACCTTA |
| | TAATCTGGGGAATTCATCACCCTTCAAGCACACAAGAAAAGAATGATCTGTATGGA |
| | ACACAATCACTTTCCATTTCAGTGGGGAGTTCTACTTATCAAAACAACTTTGTGCC |
| | TGTGGTGGGAGCAAGACCACAAGTGAATGGCCAAAGTGGGGGGATTGATTTCCA |
| | TTGGACGATGGTACAACCGGGTGATAACATCACTTTTTCGCATAATGGTGGATTAA |
| | TAGCACCTAGCAGAGTGAGTAAGCTAAAGGGAAGAGGCCTCGGCATTCAATCAG |
| | GAGCTTCAGTAGACAATGACTGT |
| 9 | Influenza A subtype H14 haemagglutinin head domain, nucleotide |
| | TGTCGGCTAAAGAATTAGTTGAGACAAACCACATTAATGAACTATGCCCAAGTCCT |
| | CTGAAGCTTGTCGATGGACAAGACTGCGACCTTATCAACGGTGCATTGGGAAGCC |
| | CAGGCTGTGACCACTTACAAGACACTACTTGGGATGTTTTCATTGAAAGGCCAAC |
| | GGCAATGGACACGTGCTATCCATTCGATGTTCCAGATTACCAGAGCCTTAGAAGC |
| | ATCTTGGCAAGCAGTGGGAGTCTGGAATTCATTGCCGAACAATTCACTTGGAATG |
| | GTGTCACAGTTGACGGATCAAGCAGTGCTTGTTTGAGGGGCGGCCGCAATGGCT |
| | TCTTCACCCGACTGAACTGGCTAACCAGAGTAAAAAACGGGAACTATGGGCCTAT |
| | TAATGTCACAAAAGAAAATACAGGGTCTTACGTCAGGCTCTATCTCTGGGGAGTG |
| | CACCATCCATCAAGTGATACTGAGCAAACGGATCTTTACAAAGTTGCAACGGGAA |
| | GAGTAACGGTGTCTACTCGCTCAGATCAAATCAGCATTATTCCCAATATAGGAAGT |
| | AGACCAAGGGTGAGGAATCAGAGCGGCAGAATAAGCATCTACTGGACTCTAGTAA |
| | ACCCAGGGGATTCCATCATCTTTAACAGCATTGGAAACCTAATTGCACCAAGAGG |
| | CCACTACAAAATAAATAAATCTACAAAGGGCACTGTGC |
| 10 | Influenza A subtype H15 haemagglutinin head domain, nucleotide |
| | TGCACAAAAGGGAAGAAAGCAGTGGACCTGGGATCTTGTGGAATACTGGGAACTA |
| | TCATTGGGCCTCCACAATGTGATCTTCATCTTGAATTCAAAGCTGATCTGATAATA |
| | GAAAGAAGAAATTCAAGTGACATCTGTTACCCAGGAAGATTCACTAATGAGGAAG |
| | CACTGAGACAAATAATCAGAGAATCTGGAGGAATTGACAAAGAGTCAATGGGCTT |
| | TAGATATTCAGGAATAAGAACAGACGGGGCAACCAGTGCGTGTAAGAGAACAGTG |
| | TCCTCTTTCTACTCAGAAATGAAATGGCTTTCATCCAGCATGAATAACCAGGTGTT |
| | CCCACAACTGAATCAGACATACAGGAACACCAGAAAAGAACCAGCCCTAATTGTC |
| | TGGGGAGTACATCATTCAAGTTCCTTGGATGAGCAAAATAAGCTATATGGAACTG |
| | GGAACAAGCTGATAACAGTAGGAAGCTCAAAGTACCAACAATCGTTTTCACCAAG |
| | TCCAGGGGCCAGGCCCAAAGTGAATGGTCAGGCCGGGAGGATCGACTTTCATTG |
| | GATGCTATTGGACCCAGGGGATACAGTCACTTTTACCTTCAATGGTGCATTCATAG |
| | CCCCAGATAGAGCCACCTTTCTCCGCTCTAATGCCCCTTCAGGAATTGAGTACAA |
| | TGGGAAGTCACTGGGAATACAGAGTGATGCACAAATCGATGAATCATGT |

Full haemagglutinin sequences

| | |
|---|---|
| 11 | Influenza A subtype H4 full haemagglutinin sequence, amino acid |
| | MLSIVILFLLAAESSSQNYTGNPVICMGHHAVANGTMVKTLTDDQVEVVTAQELVESQ |
| | NLPELCPSPLRLIDGQACDIINGALGSPGCDHLNGAEWDVFIERPNAVDTCYPFDIPDY |
| | QSLRSILANNGKFEFIAEEFSWTTVKQNGKSGACKRANVNDFFNRLNWLTKSDGNAY |
| | PLQNLTKVNNGDYARLYIWGVHHPSTDAEQTNLYKNNPGRVTVSTKTDQTSVVPNIG |
| | SRPWVRGQSGRISFYWTIVEPGDLIVFNTIGNLIAPRGHYKLNSQKKSTILNTAIPIGSC |
| | VSKCHTDKGSLSTTKPFQNISRIAIGDCPKYVKQGSLKLATGMRNIPEKASRGLFGAIA |
| | GFIENGWQGLIDGWYGFRHQNAEGTGTAADLKSTQAAIDQINGKLNRLIEKTNEKYH |
| | QIEKEFEQVEGRIQDLEKYVEDTKVDLWSYNAELLVALENQHTIDVTDSEMNKLFERV |
| | RRQLRENAEDKGNGCFEIFHKCDNNCIESIRNGTYDHNIYRDEAINNRFQIQGVKLTQ |
| | GYKDIILWISFSISCFLIVALLLAFVLWACQNGNIRCQICIXRX |
| 12 | Influenza A subtype H7 full haemagglutinin sequence, amino acid |
| | GYKMNTQILALVACMLIGAKGDKICLGHHAVANGTKVNTLTERGIEVVNATETVETANI |
| | KKICTQGKRSTDLGQCGLLGTLIGPPQCDQFLEFNADLIIERREGTDVCYPGKFTNEE |
| | SLRQILRGSGGIDKESMGFTYSGIRTNGATSACRRSGSSFYAEMKWLLSNSDNAAFP |
| | QMTKSYRNPRNKPALIIWGVHHSGSTTEQTKLYGSGNKLITVGSSKYQQSFTPSPGA |
| | RPQVNGQSGRIDFHWLLLDPNDTVTFTFNGAFIAPDRASFFRGESLGVQSDVPLDSG |
| | CEGDCFHNGGTIVSSLPFQNVNPRTVGKCPRYVKQTSLLLATGMRNVPENPKTRGLF |
| | GAIAGFIENGWEGLIDGWYGFRHQNAQGEGTAADYKSTQSAIDQITGKLNRLIDKTNQ |
| | QFELIDNEFSEIEQQIGNVINWTRDSMTEVWSYNAELLVAMENQHTIDLADSEMNKLY |
| | ERVRKQLRENAEEDGTGCFEIFHKCDDQCMESIRNNTYDHTQYRAESLQNRIQIDPV |
| | KLSSGYKDIILWFSFGASCFLLLAIAMGLVFICIKNGNMRCTICIXFEKNX |
| 13 | Influenza A subtype H10 full haemagglutinin sequence, amino acid |
| | KAGVTMYKIVLVLALFGAVHGLDKICLGHHAVSNGTIVKTLTNEKEEVTNATETVESKS |
| | LDKLCMKSRNYKDLGNCHPIGMIIGTPACDLHLTGTWDTLIERNNSIAYCYPGATVNEE |
| | ALRQKIMESGGIDKISTGFTYGSSINSAGTTAACMRNGGNSFYAELKWLVSKSKGQNF |
| | PQTTNTYRNTDSAEHLIIWGIHHPSSTQEKNDLYGTQSLSISVGSSTYQNNFVPVVGA |
| | RPQVNGQSGRIDFHWTMVQPGDNITFSHNGGLIAPSRVSKLKGRGLGIQSGASVDND |
| | CESKCFWKGGSINTKLPFQNLSPRTVGQCPKYVNKKSLLLATGMRNVPEVVQGRGL |
| | FGAIAGFIENGWEGMVDGWYGFRHQNAQGTGQAADYKSTQAAIDQITGKLNRLIEKT |
| | NTEFESIESEFSEIEHQIGNVINWTKDSITDIWTYQAELLVAMENQHTIDMADSEMLNLY |
| | ERVRKQLRQNAEEDGKGCFEIYHKCDDNCMESIRNNTYDHTQYREEALLNRLNINPV |
| | KLSSGYKDVILWFSFGASCFVLLAVIMGLVFFCLKNGNMRCTICIXLKTPLFLX |
| 14 | Influenza A subtype H14 full haemagglutinin sequence, amino acid |
| | SKSRGKXLHSXXLHWLXATLLTPRSQMETQETLLYAWVTMQWRMAHLLKHXQTIIXR |
| | LCRLKNXLRQTTLMNYAQVLXSLSMDKTATLSTVHWEAQAVTTYKTLLGMFSLKGQR |
| | QWTRAIHSMFQITRALEASWQAVGVWNSLPNNSLGMVSQLTDQAVLVXGAAAMASS |
| | PDXTGXPEXKTGTMGLLMSQKKIQGLTSGSISGECTIHQVILSKRIFTKLQREEXRCLL |
| | AQIKSALFPIXEVDQGXGIRAAEXASTGLXXTQGIPSSLTALETXLHQEATTKXINLQRA |
| | LCLKVTRRLDHAQALVXPIKVQSKVTNLFRMCPESLXETARSMXSKAHXCWQLECVT |
| | SLTSRRRAYLEQLLDSLKMVGKAXLMGGMDSGTKMLREQELLRIXNQPRQPLTKXMA |
| | NXTDXXRRQMKNITKSKKNSSKWKEEYRTLRNTLRTLKLICGHTMLSYWWPXKISTQ |
| | XMLQTPRXTNSLRGXEDNLGKMRKIKATDVSRYSISVTTTVXKVSEMEPMITTSTGMK |
| | PLTIGLKXIPSIXRWVTRTXYCGFLSPCHALSLWHXFWDLFFGLVRTEISDAKFVYKEK |
| | TPLFLX |
| 15 | Influenza A subtype H15 full haemagglutinin sequence, amino acid |
| | SKSRGYKMNTQIIVILVLGLSMVKSDKICLGHHAVANGTKVNTLTERGVEVVNATETVE |
| | ITGIDKVCTKGKKAVDLGSCGILGTIIGPPQCDLHLEFKADLIIERRNSSDICYPGRFTNE |
| | EALRQIIRESGGIDKESMGFRYSGIRTDGATSACKRTVSSFYSEMKWLSSSMNNQVF |
| | PQLNQTYRNTRKEPALIVWGVHHSSSLDEQNKLYGTGNKLITVGSSKYQQSFSPSPG |
| | ARPKVNGQAGRIDFHWMLLDPGDTVTFTFNGAFIAPDRATFLRSNAPSGIEYNGKSL |
| | GIQSDAQIDESCEGECFYSGGTINSPLPFQNIDSRAVGKCPRYVKQSSLPLALGMKNV |
| | PEKIRTRGLFGAIAGFIENGWEGLIDGWYGFRHQNAQGQGTAADYKSTQAAIDQITGK |
| | LNRLIEKTNKQFELIDNEFTEVEQQIGNVINWTRDSLTEIWSYNAELLVAMENQHTIDLA |
| | DSEMNKLYERVRRQLRENAEEDGTGCFEIFHRCDDQCMESIRNNTYNHTEYRQEAL |
| | QNRIMINPVKLSSGYKDVILWFSFGASCVMLLAIAMGLIFMCVKNGNLRCTICIXLFEKH |
| | PCFYX |
| 16 | Influenza A subtype H4 full haemagglutinin sequence, nucleotide |
| | ATGCTATCAATTGTAATTCTGTTTCTGCTCGCGGCAGAGAGCTCCTCCCAAAACTA |
| | CACAGGAAACCCTGTGATATGCATGGGGCATCATGCCGTTGCCAATGGAACCATG |
| | GTAAAGACACTAACCGATGACCAAGTGGAGGTGGTCACTGCACAGGAATTGGTAG |
| | AATCACAGAATCTCCCGGAATTGTGCCCGAGTCCTCTGAGATTGATTGACGGCCA |
| | GGCTTGCGACATCATAAATGGGGCATTGGGAAGCCCAGGGTGTGACCATCTGAA |
| | TGGTGCAGAATGGGACGTGTTCATAGAAAGGCCCAATGCAGTGGACACCTGTTAC |
| | CCCTTCGATATTCCAGACTATCAGAGTTTGAGAAGCATACTTGCTAACAATGGGAA |
| | GTTTGAATTCATTGCTGAAGAATTTTCATGGACCACTGTAAAACAAAATGGAAAGT |
| | CTGGAGCCTGCAAAAGGGCGAATGTGAATGATTTCTTTAATAGGCTGAATTGGCT |
| | GACAAAGTCAGACGGAAATGCCTATCCTCTTCAAAATTTGACGAAAGTAAATAATG |
| | GTGATTACGCAAGACTTTACATCTGGGGAGTTCATCACCCTTCAACAGACGCAGA |
| | GACCAAACAAGTGTAGTCCCTAATATTGGCAGTAGGCCCTGGGTAAGAGGGCAAA |
| | GCGGGAGAATAAGCTTCTACTGGACCATTGTGGAGCCCGGGGACCTGATAGTCT |
| | TCAACACAATAGGAAACCTAATTGCCCCAAGAGGGCATTACAAACTGAATAGCCA |
| | GAAAAAAAGCACAATTCTAAATACTGCAATACCAATAGGCTCATGTGTTAGCAAAT |
| | GTCACACAGACAAAGGCTCTCTATCCACAACCAAACCTTTCCAGAATATCTCGAGA |
| | ATAGCAATTGGAGATTGCCCCAAATATGTTAAGCAGGGCTCTTTAAAACTTGCCAC |
| | AGGAATGAGAAATATTCCGGAGAAGGCATCAAGAGGGCTCTTTGGTGCAATAGCT |
| | GGGTTCATAGAGAATGGGTGGCAAGGTCTAATTGATGGATGGTATGGGTTCAGGC |
| | ACCAGAATGCAGAAGGAACAGGGACAGCAGCAGACCTGAAATCCACTCAAGCAG |
| | CCATTGACCAGATCAATGGGAAGTTGAACCGTCTTATCGAGAAGACAAACGAAAA |
| | ATACCATCAAATCGAAAAGGAGTTCGAACAGGTTGAAGGGAGAATTCAAGATCTG |
| | GAGAAGTATGTAGAGGATACCAAGGTTGATTTATGGTCATATAATGCAGAGTTGTT |
| | GGTCGCACTGGAGAACCAGCACACTATTGATGTAACTGACTCAGAGATGAATAAA |
| | CTTTTTGAGAGAGTAAGGCGCCAACTCAGAGAAAATGCTGAAGACAAAGGAAATG |
| | GGTGTTTCGAGATATTCCACAAGTGTGATAACAATTGCATTGAAAGCATCCGGAAT |
| | GGAACTTACGATCATAACATTTATCGAGATGAGGCAATCAACAACCGATTCCAAAT |
| | TCAGGGAGTTAAATTGACTCAGGGGTACAAGGACATTATTCTCTGGATTTCATTCT |
| | CCATATCATGCTTTTTGATCGTCGCACTACTTTTGGCCTTCGTTTTGTGGGCTTGT |
| | CAAAACGGAAACATCCGGTGCCAGATTTGCATTTGAAGAA |
| 17 | Influenza A subtype H7 full haemagglutinin sequence, nucleotide |
| | GGATACAAAATGAACACTCAAATTTTGGCACTCGTTGCTTGTATGCTGATTGGAGC |
| | AAAAGGAGATAAAATATGTCTTGGGCATCATGCTGTGGCAAATGGAACAAAGGTA |
| | AACACATTAACAGAGAGAGGGATCGAAGTAGTAAATGCCACAGAAACGGTGGAAA |
| | CCGCGAATATTAAGAAAATATGCACTCAGGGGAAAAGATCAACAGATCTGGGACA |
| | ATGCGGACTTCTAGGAACCCTAATAGGACCTCCCCAATGCGATCAATTTTTGGAG |
| | TTTAACGCTGATTTAATAATTGAACGAAGAGAAGGAACCGATGTGTGTTATCCCGG |
| | GAAATTCACAAATGAAGAATCACTGAGGCAGATCCTTCGAGGATCAGGAGGAATT |
| | GATAAGGAGTCAATGGGCTTCACCTATAGTGGAATAAGAACCAATGGAGCGACGA |
| | GTGCTTGCAGAAGATCAGGTTCTTCCTTCTATGCGGAGATGAAGTGGTTGCTGTC |
| | GAATTCAGACAATGCGGCTTTTCCCCAAATGACCAAATCGTATAGAAATCCCAGGA |
| | ACAAACCAGCTCTAATAATTTGGGGAGTGCATCACTCTGGATCGACTACTGAGCA |
| | AACTAAACTCTATGGGAGTGGAAACAAGTTGATAACAGTGGGAAGCTCGAAATAC |
| | CAGCAGTCATTCACCCCAAGCCCGGGGGCACGACCACAAGTGAATGGGCAATCA |
| | GGAAGGATTGATTTTCATTGGCTACTCCTTGATCCCAATGACACAGTGACTTTCAC |
| | TTTCAATGGGGCATTCATAGCTCCTGACAGAGCAAGTTTCTTTAGAGGAGAATCAC |
| | TAGGAGTTCAGAGTGATGTTCCTTTGGATTCTGGTTGTGAGGGGGATTGCTTCCA |
| | CAATGGGGGTACGATAGTGAGCTCCCTGCCATTCCAGAATGTCAACCCTAGAACA |
| | GTGGGAAAATGCCCTCGATATGTCAAACAGACAAGCCTCCTTTTGGCTACAGGGA |
| | TGAGAAACGTCCCAGAGAACCCCAAGACCAGAGGCCTTTTTGGAGCGATCGCTG |
| | GATTCATAGAGAATGGATGGGAAGGTCTCATTGATGGATGGTATGGTTTCAGACA |
| | TCAAAATGCACAAGGAGAAGGAACAGCAGCTGATTACAAAAGCACTCAATCCGCT |
| | ATAGATCAGATAACAGGCAAATTGAATCGTCTAATTGACAAAACAAATCAGCAGTT |
| | TGAACTGATAGACAACGAATTCAGTGAAATAGAACAACAAATTGGGAATGTCATTA |
| | ACTGGACAAGAGATTCAATGACTGAGGTATGGTCGTACAATGCTGAATTGCTGGT |
| | AGCTATGGAAAATCAGCACACAATAGATCTTGCAGACTCAGAAATGAACAAACTTT |
| | ATGAGCGTGTAAGGAAACAACTGAGGGAAAATGCTGAAGAGGATGGGACTGGAT |
| | GCTTTGAGATATTCCATAAGTGTGATGATCAGTGCATGGAGAGCATAAGGAACAA |
| | CACTTATGATCACACTCAATACAGAGCAGAGTCATTGCAGAATAGAATACAGATAG |
| | ACCCAGTGAAATTGAGTAGTGGATACAAAGACATAATCTTATGGTTTAGCTTCGGG |
| | GCATCATGTTTTCTTCTTCTAGCCATTGCAATGGGATTGGTTTTCATTTGCATAAAG |
| | AATGGAAACATGCGGTGCACTATTTGTATATAGTTTGAGAAAAACAC |
| 18 | Influenza A subtype H10 full haemagglutinin sequence, nucleotide |
| | AAAGCAGGGGTCACAATGTACAAAATAGTACTAGTACTCGCGCTCTTTGGAGCGG |
| | TGCATGGTCTTGACAAAATATGTCTTGGACATCATGCAGTCTCCAATGGCACCATC |
| | GTAAAGACTCTCACAAACGAAAAGGAAGAGGTGACCAATGCTACTGAAACAGTGG |
| | AGAGCAAAAGCCTGGACAAACTTTGCATGAAAAGTCGGAATTACAAGGACTTAGG |
| | TAATTGTCACCCAATAGGGATGATAATAGGGACTCCTGCTTGTGATCTACACCTCA |
| | CCGGAACATGGGACACTTTGATAGAGAGAAACAATTCTATTGCCTACTGTTACCC |
| | GGGTGCCACTGTGAATGAAGAAGCATTAAGGCAGAAAATTATGGAAAGTGGAGG |
| | GATTGACAAGATAAGCACCGGGTTTACATATGGATCATCTATCAATTCGGCTGGAA |
| | CCACTGCAGCATGCATGAGAAATGGAGGAAATAGTTTCTATGCGGAGCTAAAGTG |
| | GCTAGTATCTAAGAGCAAAGGACAAAACTTCCCACAAACAACAAACACATACAGAA |
| | ATACAGATTCAGCAGAACACCTTATAATCTGGGGAATTCATCACCCTTCAAGCACA |
| | CAAGAAAAGAATGATCTGTATGGAACACAATCACTTTCCATTTCAGTGGGGAGTTC |
| | TACTTATCAAAACAACTTTGTGCCTGTGGTGGGAGCAAGACCACAAGTGAATGGC |
| | CAAAGTGGGGGGATTGATTTCCATTGGACGATGGTACAACCGGGTGATAACATCA |
| | CTTTTTCGCATAATGGTGGATTAATAGCACCTAGCAGAGTGAGTAAGCTAAAGGG |
| | AAGAGGCCTCGGCATTCAATCAGGAGCTTCAGTAGACAATGACTGTGAGTCAAAA |
| | TGTTTTTGGAAAGGTGGATCCATCAACACCAAACTCCCTTTCCAGAATCTTTCTCC |
| | AAGAACTGTGGGTCAATGCCCCAAGTATGTGAACAAAAAGAGCCTGTTGCTTGCT |
| | ACTGGAATGAGGAATGTGCCAGAGGTTGTCCAAGGAAGAGGCCTGTTTGGAGCA |
| | ATAGCTGGATTCATAGAAAATGGATGGGAAGGAATGGTAGATGGTTGGTATGGTT |
| | TCCGGCATCAAAATGCCCAAGGCACTGGCCAGGCCGCGGATTACAAAAGTACTC |
| | AGGCAGCTATAGATCAAATAACCGGGAAATTGAACAGGCTGATAGAGAAGACAAA |
| | CACAGAGTTCGAATCCATAGAATCTGAATTCAGTGAAATAGAACATCAAATCGGCA |
| | ATGTAATAAACTGGACTAAGGATTCCATAACAGACATCTGGACGTATCAAGCTGAA |
| | TTGCTGGTAGCAATGGAAAATCAGCATACAATTGACATGGCTGATTCAGAAATGCT |
| | GAATCTATATGAGAGGGTGAGGAAGCAACTAAGGCAGAATGCTGAAGAAGATGG |
| | GAAAGGGTGCTTTGAAATATATCACAAATGCGATGACAACTGTATGGAAAGCATCA |
| | GAAACAACACTTATGACCATACACAATACAGAGAAGAAGCACTCTTGAACAGACTC |
| | AACATCAATCCTGTGAAACTCTCTTCTGGGTACAAAGATGTTATACTGTGGTTTAG |
| | CTTCGGGGCGTCATGCTTCGTACTTTTGGCTGTCATCATGGGGCTTGTTTTCTTCT |
| | GCTTGAAAAATGGAAACATGCGATGCACAATCTGTATTTAGTTAAAAACACCCTTG |
| | TTTCTAC |
| 19 | Influenza A subtype H14 full haemagglutinin sequence, nucleotide |
| | AGCAAAAGCAGGGGAAAATGATTGCATTCATAGTGATTGCACTGGCTCTGAGCCA |
| | CACTACTTACTCCCAGATCACAAATGGAAACACAGGAAACCCTGTTATATGCTTGG |
| | GTCACCATGCAGTGGAGAATGGCACATCTGTTAAAACATTAACAGACAATCATATA |
| | GAGGTTGTGTCGGCTAAAGAATTAGTTGAGACAAACCACATTAATGAACTATGCCC |
| | AAGTCCTCTGAAGCTTGTCGATGGACAAGACTGCGACCTTATCAACGGTGCATTG |
| | GGAAGCCCAGGCTGTGACCACTTACAAGACACTACTTGGGATGTTTTCATTGAAA |
| | GGCCAACGGCAATGGACACGTGCTATCCATTCGATGTTCCAGATTACCAGAGCCT |
| | TAGAAGCATCTTGGCAAGCAGTGGGAGTCTGGAATTCATTGCCGAACAATTCACT |
| | TGGAATGGTGTCACAGTTGACGGATCAAGCAGTGCTTGTTTGAGGGGCGGCCGC |
| | AATGGCTTCTTCACCCGACTGAACTGGCTAACCAGAGTAAAAAACGGGAACTATG |
| | GGCCTATTAATGTCACAAAAGAAAATACAGGGTCTTACGTCAGGCTCTATCTCTGG |
| | GGAGTGCACCATCCATCAAGTGATACTGAGCAAACGGATCTTTACAAAGTTGCAA |
| | CGGGAAGAGTAACGGTGTCTACTCGCTCAGATCAAATCAGCATTATTCCCAATATA |
| | GGAAGTAGACCAAGGGTGAGGAATCAGAGCGGCAGAATAAGCATCTACTGGACT |
| | CTAGTAAACCCAGGGGATTCCATCATCTTTAACAGCATTGGAAACCTAATTGCACC |
| | AAGAGGCCACTACAAAATAAATAAATCTACAAAGGGCACTGTGCTTAAAAGTGACA |
| | AGAAGATTGGATCATGCACAAGCCCTTGTTTAACCGATAAAGGTTCAATCCAAAGT |
| | GACAAACCTTTTCAGAATGTGTCCAGAATCGCTATAGGAAACTGCCCGAAGTATGT |
| | GAAGCAAGGCTCACTGATGTTGGCAACTGGAATGCGTAACATCCCTGACAAGCAG |
| | ACGAAGGGCTTATTTGGAGCAATTGCTGGATTCATTGAAAATGGTTGGCAAGGCC |
| | TGATTGATGGGTGGTATGGATTCAGGCACCAAAATGCTGAGGGAACAGGAACTGC |
| | TGCGGATTTGAAATCAACCCAGGCAGCCATTGACCAAATAAATGGCAAACTAAAC |
| | AGATTGATAGAGAAGACAAATGAAAAATATCACCAAATCGAAAAAGAATTCGAGCA |
| | AGTGGAAGGAAGAATACAGGACCTTGAGAAATACGTTGAGGACACTAAAATTGAT |
| | CTGTGGTCATACAATGCTGAGCTATTGGTGGCCCTAGAAAATCAGCACACAATAG |
| | ATGTTACAGACTCCGAGATGAACAAACTCTTTGAGAGGGTGAGAAGACAACTTAG |
| | GGAAAATGCGGAAGATCAAGGCAACGGATGTTTCGAGATATTCCATCAGTGTGAC |
| | AACAACTGTATAGAAAGTATCAGAAATGGAACCTATGATCACAACATCTACAGGGA |
| | TGAAGCCATTAACAATCGGATTAAAATAAATCCCGTCAATTTGACGATGGGTTACA |
| | AGGACATAATACTGTGGATTTCTTTCTCCATGTCATGCTTTGTCTTTGTGGCACTG |
| | ATTTTGGGATTTGTTCTTTGGGCTTGTAAGAACGGAAATATCCGATGCCAAATTTG |
| | TATATAAAGAAAAAACACCCTTGTTTCTACT |
| 20 | Influenza A subtype H15 full haemagglutinin sequence, nucleotide |
| | AGCAAAAGCAGGGGATACAAAATGAACACTCAAATCATTGTCATTCTAGTCCTCGG |
| | ACTGTCAATGGTGAAATCTGACAAGATTTGTCTCGGGCACCATGCCGTAGCAAAT |
| | GGGACAAAAGTCAACACACTAACTGAGAGAGGAGTGGAAGTGGTCAATGCCACG |
| | GAGACAGTGGAGATTACCGGAATAGATAAAGTGTGCACAAAAGGGAAGAAAGCA |
| | GTGGACCTGGGATCTTGTGGAATACTGGGAACTATCATTGGGCCTCCACAATGTG |
| | ATCTTCATCTTGAATTCAAAGCTGATCTGATAATAGAAAGAAGAAATTCAAGTGACA |
| | TCTGTTACCCAGGAAGATTCACTAATGAGGAAGCACTGAGACAAATAATCAGAGA |
| | ATCTGGAGGAATTGACAAAGAGTCAATGGGCTTTAGATATTCAGGAATAAGAACA |
| | GACGGGGCAACCAGTGCGTGTAAGAGAACAGTGTCCTCTTTCTACTCAGAAATGA |
| | AATGGCTTTCATCCAGCATGAATAACCAGGTGTTCCCACAACTGAATCAGACATAC |
| | AGGAACACCAGAAAAGAACCAGCCCTAATTGTCTGGGGAGTACATCATTCAAGTT |
| | CCTTGGATGAGCAAAATAAGCTATATGGAACTGGGAACAAGCTGATAACAGTAGG |
| | AAGCTCAAAGTACCAACAATCGTTTTCACCAAGTCCAGGGGCCAGGCCCAAAGTG |
| | AATGGTCAGGCCGGGAGGATCGACTTTCATTGGATGCTATTGGACCCAGGGGAT |
| | ACAGTCACTTTTACCTTCAATGGTGCATTCATAGCCCCAGATAGAGCCACCTTTCT |
| | CCGCTCTAATGCCCCTTCAGGAATTGAGTACAATGGGAAGTCACTGGGAATACAG |
| | AGTGATGCACAAATCGATGAATCATGTGAAGGGGAATGCTTCTACAGTGGAGGGA |
| | CAATAAACAGCCCTTTACCATTTCAAAACATCGATAGTAGGGCTGTCGGAAAGTGC |
| | CCCAGATATGTGAAGCAATCAAGCTTGCCGCTGGCCTTAGGAATGAAAAATGTAC |
| | CAGAGAAAATACGTACTAGGGGACTGTTCGGTGCAATTGCAGGATTCATCGAAAA |
| | TGGATGGGAAGGGCTCATTGATGGATGGTATGGATTTAGGCATCAGAATGCACAA |
| | GGGCAGGGAACAGCTGCTGACTACAAGAGTACTCAGGCTGCAATTGACCAGATA |
| | ACAGGGAAACTTAATAGGTTAATTGAAAAAACCAACAAACAGTTTGAACTCATAGA |
| | CAATGAGTTCACTGAAGTGGAGCAGCAGATAGGCAATGTAATAAACTGGACAAGG |
| | GACTCCTTGACTGAGATCTGGTCATACAATGCCGAACTGCTAGTAGCAATGGAGA |
| | ATCAGCATACAATTGACCTTGCAGATTCTGAAATGAACAAACTCTATGAGAGAGTG |
| | AGAAGACAGCTAAGGGAGAATGCCGAGGAGGATGGAACTGGATGTTTTGAGATTT |
| | TCCACCGATGTGACGATCAATGTATGGAGAGCATACGGAATAATACTTACAATCAC |
| | ACTGAATATCGACAGGAAGCCTTACAAAATAGGATAATGATCAATCCGGTAAAGCT |
| | TAGTAGTGGGTACAAAGATGTGATACTATGGTTTAGCTTCGGGGCATCATGTGTAA |
| | TGCTTCTAGCCATTGCTATGGGTCTTATTTTCATGTGTGTGAAAAACGGGAATCTG |
| | CGGTGCACTATCTGTATATAATTATTTGAAAAACACCCTTGTTTCTACT |

Epitope 1 (MAIZ) sequences in preferred scaffolds
(specified amino acids are bold/underlined)

| | |
|---|---|
| 21 | Omega epitope in influenza A subtype H4 haemagglutinin head domain, amino |
| | acid |
| | CPSPLRLIDGQACDIINGALGSPGCDHLNGAEWDVFIERPNAVDTCYPFDIPDYQSLR |
| | SILANNGKFEFIAEEFSWTTVKQNGKSGACIRRSSSSFFSRLNWLTKSDGNAYPLQNL |
| | TKVNNGDYARLYIWGVHHPSTDAEQTNLYKNNPGRVTVSTKTDQTSVVPNIGSRPW |
| | VRGQSGRISFYWTIVEPGDLIVFNTIGNLIAPRGHYKLNSQKKSTILNTAIPIGSC |
| 22 | Tau epitope in influenza A subtype H4 haemagglutinin head domain, amino acid |
| | CPSPLRLIDGQACDIINGALGSPGCDHLNGAEWDVFIERPNAVDTCYPFDIPDYQSLR |
| | SILANNGKFEFIAEEFSWTTVKQNGKSGACKRRSNNSFFSRLNWLTKSDGNAYPLQN |
| | LTKVNNGDYARLYIWGVHHPSTDAEQTNLYKNNPGRVTVSTKTDQTSVVPNIGSRPW |
| | VRGQSGRISFYWTIVEPGDLIVFNTIGNLIAPRGHYKLNSQKKSTILNTAIPIGSC |
| 23 | Sigma epitope in influenza A subtype H4 haemagglutinin head domain, amino |
| | acid |
| | CPSPLRLIDGQACDIINGALGSPGCDHLNGAEWDVFIERPNAVDTCYPFDIPDYQSLR |
| | SILANNGKFEFIAEEFSWTTVKQNGKSGACKRRSNKSFFSRLNWLTKSDGNAYPLQN |
| | LTKVNNGDYARLYIWGVHHPSTDAEQTNLYKNNPGRVTVSTKTDQTSVVPNIGSRPW |
| | VRGQSGRISFYWTIVEPGDLIVFNTIGNLIAPRGHYKLNSQKKSTILNTAIPIGSC |
| 24 | Rho epitope in influenza A subtype H4 haemagglutinin head domain, amino acid |
| | CPSPLRLIDGQACDIINGALGSPGCDHLNGAEWDVFIERPNAVDTCYPFDIPDYQSLR |
| | SILANNGKFEFIAEEFSWTTVKQNGKSGACKRGSVKSFFSRLNWLTKSDGNAYPLQN |
| | LTKVNNGDYARLYIWGVHHPSTDAEQTNLYKNNPGRVTVSTKTDQTSVVPNIGSRPW |
| | VRGQSGRISFYWTIVEPGDLIVFNTIGNLIAPRGHYKLNSQKKSTILNTAIPIGSC |
| 25 | Mu epitope in influenza A subtype H4 haemagglutinin head domain, amino acid |
| | CPSPLRLIDGQACDIINGALGSPGCDHLNGAEWDVFIERPNAVDTCYPFDIPDYQSLR |
| | SILANNGKFEFIAEEFSWTTVKQNGKSGACKRGSVNSFFSRLNWLTKSDGNAYPLQN |
| | LTKVNNGDYARLYIWGVHHPSTDAEQTNLYKNNPGRVTVSTKTDQTSVVPNIGSRPW |
| | VRGQSGRISFYWTIVEPGDLIVFNTIGNLIAPRGHYKLNSQKKSTILNTAIPIGSC |
| 26 | Theta epitope in influenza A subtype H4 haemagglutinin head domain, amino |
| | acid |
| | CPSPLRLIDGQACDIINGALGSPGCDHLNGAEWDVFIERPNAVDTCYPFDIPDYQSLR |
| | SILANNGKFEFIAEEFSWTTVKQNGKSGACKRGSDNSFFSRLNWLTKSDGNAYPLQN |
| | LTKVNNGDYARLYIWGVHHPSTDAEQTNLYKNNPGRVTVSTKTDQTSVVPNIGSRPW |
| | VRGQSGRISFYWTIVEPGDLIVFNTIGNLIAPRGHYKLNSQKKSTILNTAIPIGSC |

Epitope 2 (INDY) sequences in preferred scaffolds
(specified amino acids are bold/underlined)

| | |
|---|---|
| 27 | Alpha epitope in influenza A subtype H4 haemagglutinin head domain, amino |
| | acid |
| | CPSPLRLIDGQACDIINGALGSPGCDHLNGAEWDVFIERPNAVDTCYPFDIPDYQSLR |
| | SILANNGKFEFIAEEFNWTGVKQNGKSGACKRANVNDFFNRLNWLTKSDGNAYPLQN |
| | LTKVNKEQFDKLYIWGVHHPSTDAEQAVLYKNNPGRVTVSTKTDQTSVVPNIGSRPW |
| | VRGQSGRISFYWTIVEPGDLLINSGNLIAPRGHYKLNSQKKSTILNTAIPIGSC |
| 28 | Beta epitope in influenza A subtype H4 haemagglutinin head domain, amino acid |
| | CPSPLRLIDGQACDIINGALGSPGCDHLNGAEWDVFIERPNAVDTCYPFDIPDYQSLR |
| | SILANNGKFEFIAEEFNWTGVKQNGKSGACKRANVNDFFNRLNWLTKSDGNAYPLQN |
| | LTKVNNEKFDKLYIWGVHHPSTDAEQTVLYKNNPGRVTVSTKTDQTSVVPNIGSRPW |
| | VRGQSGRISFYWTIVEPGDLLINSGNLIAPRGHYKLNSQKKSTILNTAIPIGSC |
| 29 | Gamma epitope in influenza A subtype H4 haemagglutinin head domain, amino |
| | acid |
| | CPSPLRLIDGQACDIINGALGSPGCDHLNGAEWDVFIERPNAVDTCYPFDIPDYQSLR |
| | SILANNGKFEFIAEEFNWTGVKQNGKSGACKRANVNDFFNRLNWLTKSDGNAYPLQN |
| | LTKVNNGKFDKLYIWGVHHPSTDAEQTVLYKNNPGRVTVSTKTDQTSVVPNIGSRPW |
| | VRGQSGRISFYWTIVEPGDLLINSGNLIAPRGHYKLNSQKKSTILNTAIPIGSC |
| 30 | Delta epitope in influenza A subtype H4 haemagglutinin head domain, amino acid |
| | CPSPLRLIDGQACDIINGALGSPGCDHLNGAEWDVFIERPNAVDTCYPFDIPDYQSLR |
| | SILANNGKFEFIAEEFNWTGVKQNGKSGACKRANVNDFFNRLNWLTKSDGNAYPLQN |
| | LTKVNNGNFDKLYIWGVHHPSTDAEQTILYKNNPGRVTVSTKTDQTSVVPNIGSRPW |
| | VRGQSGRISFYWTIVEPGDLLINSGNLIAPRGHYKLNSQKKSTILNTAIPIGSC |
| 31 | Reference sequence |
| | NCBI Accession AY661004.1; Influenza A virus; A/Bilthoven/3517/1973(H3N2)) |
| | 1 mktiialsyi fclvlgqdfp gndnstatlc lghhavpngt lvktitndqi evtnatelvq |
| | 61 ssstgkicnn phrildgidc tlidallgdp hcdgfqnetw dlfverskaf sncypydvpd |
| | 121 yaslrslvas sgtlefineg ftwtgvtqng gsnackrgtd sgffsrlnwl yksgstypvl |
| | 181 nvtmpnndnf dklyiwgvhh pstdqeqtsl yvqasgrvtv stkrsqqtii pnigsrpwvr |
| | 241 glssrisiyw tivkpgdilv insngnliap rgyfkmrtgk ssimrsdapi gtcisecitp |
| | 301 ngsipndkpf qnvnkityga cpkyvkqntl klatgmrnvp ekqtrglfga iagfiengwe |
| | 361 gmi |

mRNA vaccine sequences (epitope sequences are underlined)

| | |
|---|---|
| 32 | Omega epitope in influenza A subtype H4 haemagglutinin head domain, |
| | nucleotide sequence |
| | UGCCCCAGCCCCCUGAGGCUGAUCGACGGCCAGGCCUGCGACAUCAUCAACGGCGCCC |
| | UGGGCAGCCCCGGCUGCGACCACCUGAACGGCGCCGAGUGGGACGUGUUCAUCGAGAG |
| | GCCCAACGCCGUGGACACCUGCUACCCCUUCGACAUCCCCGACUACCAGAGCCUGAGG |
| | AGCAUCCUGGCCAACAACGGCAAGUUCGAGUUCAUCGCCGAGGAGUUCAGCUGGACCA |
| | CCGUGAAGCAGAACGGCAACGGCACCAGCAGCGCCUGCAUCAGGAGGAGCAGCAGCAG |
| | CUUCUUCAGCAGGCUGAACUGGCUGACCAAGAGCGACGGCAACGCCUACCCCCUGCAG |
| | AACCUGACCAAGGUGAACAACGGCGACUACGCCAGGCUGUACAUCUGGGGCGUGCACC |
| | ACCCCAGCACCGACGCCGAGCAGACCAACCUGUACAAGAACAACCCCGGCAGGGUGAC |
| | CGUGAGCACCAAGACCGACCAGACCAGCGUGGUGCCCAACAUCGGCAGCAGGCCCUGG |
| | GUGAGGGGCCAGAGCGGCAGGAUCAGCUUCUACUGGACCAUCGUGGAGCCCGGCGACC |
| | UGAUCGUGUUCAACACCAUCGGCAACCUGAUCGCCCCCAGGGGCCACUACAAGCUGAA |
| | CAGCCAGAAGAAGAGCACCAUCCUGAACACCGCCAUCCCCAUCGGCAGCUGC |
| 33 | Omega epitope in influenza A subtype H7 haemagglutinin head domain, |
| | nucleotide sequence |
| | UGCACCCAGGGCAAGAGGAGCACCGACCUGGGCCAGUGCGGCCUGCUGGGCACCCUGA |
| | UCGGCCCCCCCCAGUGCGACCAGUUCCUGGAGUUCAACGCCGACCUGAUCAUCGAGAG |
| | GAGGGAGGGCACCGACGUGUGCUACCCCGGCAAGUUCACCAACGAGGAGAGCCUGAGG |
| | CAGAUCCUGAGGGGCAGCGGCGGCAUCGACAAGGAGAGCAUGGGCUUCACCUACAGCG |
| | GCAUCAGGACCAACGGCACCAGCAGCGCCUGCAUCAGGAGGAGCAGCAGCAGCUUCUU |
| | CAGCAGGCUGAACUGGCUGCUGAGCAACAGCGACAACGCCGCCUUCCCCCAGAUGACC |
| | AAGAGCUACAGGAACCCCAGGAACAAGCCCGCCCUGAUCAUCUGGGGCGUGCACCACA |
| | GCGGCAGCACCACCGAGCAGACCAAGCUGUACGGCAGCGGCAACAAGCUGAUCACCGU |
| | GGGCAGCAGCAAGUACCAGCAGAGCUUCACCCCCAGCCCCGGCGCCAGGCCCCAGGUG |
| | AACGGCCAGAGCGGCAGGAUCGACUUCCACUGGCUGCUGCUGGACCCCAACGACACCG |
| | UGACCUUCACCUUCAACGGCGCCUUCAUCGCCCCCGACAGGGCCAGCUUCUUCAGGGG |
| | CGAGAGCCUGGGCGUGCAGAGCGACGUGCCCCUGGACAGCGGCUGCGAGGGCGACUGC |
| 34 | Omega epitope in influenza A subtype H10 haemagglutinin head domain, |
| | nucleotide sequence |
| | UGCAUGAAGAGCAGGAACUACAAGGACCUGGGCAACUGCCACCCCAUCGGCAUGAUCA |
| | UCGGCACCCCCGCCUGCGACCUGCACCUGACCGGCACCUGGGACACCCUGAUCGAGAG |
| | GAACAACAGCAUCGCCUACUGCUACCCCGGCGCCACCGUGAACGAGGAGGCCCUGAGG |
| | CAGAAGAUCAUGGAGAGCGGCGGCAUCGACAAGAUCAGCACCGGCUUCACCUACGGCA |
| | GCAGCAUCAACAGCAACGGCACCAGCAGCGCCUGCAUCAGGAGGAGCAGCAGCAGCUU |
| | CUUCAGCAGGCUGAACUGGCUGGUGAGCAAGAGCAAGGGCCAGAACUUCCCCCAGACC |
| | ACCAACACCUACAGGAACACCGACAGCGCCGAGCACCUGAUCAUCUGGGGCAUCCACC |
| | ACCCCAGCAGCACCCAGGAGAAGAACGACCUGUACGGCACCCAGAGCCUGAGCAUCAG |
| | CGUGGGCAGCAGCACCUACCAGAACAACUUCGUGCCCGUGGUGGGCGCCAGGCCCCAG |
| | GUGAACGGCCAGAGCGGCAGGAUCGACUUCCACUGGACCAUGGUGCAGCCCGGCGACA |
| | ACAUCACCUUCAGCCACAACGGCGGCCUGAUCGCCCCCAGCAGGGUGAGCAAGCUGAA |
| | GGGCAGGGGCCUGGGCAUCCAGAGCGGCGCCAGCGUGGACAACGACUGC |
| 35 | Omega epitope in influenza A subtype H15 haemagglutinin head domain, |
| | nucleotide sequence |
| | UGCACCAAGGGCAAGAAGGCCGUGGACCUGGGCAGCUGCGGCAUCCUGGGCACCAUCA |
| | UCGGCCCCCCCCAGUGCGACCUGCACCUGGAGUUCAAGGCCGACCUGAUCAUCGAGAG |
| | GAGGAACAGCAGCGACAUCUGCUACCCCGGCAGGUUCACCAACGAGGAGGCCCUGAGG |
| | CAGAUCAUCAGGGAGAGCGGCGGCAUCGACAAGGAGAGCAUGGGCUUCAGGUACAGCG |
| | GCAUCAGGACCAACGGCACCAGCAGCGCCUGCAUCAGGAGGAGCAGCAGCAGCUUCUU |
| | CAGCAGGCUGAACUGGCUGAGCAGCAGCAUGAACAACCAGGUGUUCCCCCAGCUGAAC |
| | CAGACCUACAGGAACACCAGGAAGGAGCCCGCCCUGAUCGUGUGGGGCGUGCACCACA |
| | GCAGCAGCCUGGACGAGCAGAACAAGCUGUACGGCACCGGCAACAAGCUGAUCACCGU |
| | GGGCAGCAGCAAGUACCAGCAGAGCUUCAGCCCCAGCCCCGGCGCCAGGCCCAAGGUG |
| | AACGGCCAGGCCGGCAGGAUCGACUUCCACUGGAUGCUGCUGGACCCCGGCGACACCG |
| | UGACCUUCACCUUCAACGGCGCCUUCAUCGCCCCCGACAGGGCCACCUUCCUGAGGAG |
| | CAACGCCCCCAGCGGCAUCGAGUACAACGGCAAGAGCCUGGGCAUCCAGAGCGACGCC |
| | CAGAUCGACGAGAGCUGC |
| 36 | Mu epitope in influenza A subtype H4 haemagglutinin head domain, nucleotide |
| | sequence |
| | UGCCCCAGCCCCCUGAGGCUGAUCGACGGCCAGGCCUGCGACAUCAUCAACGGCGCCC |
| | UGGGCAGCCCCGGCUGCGACCACCUGAACGGCGCCGAGUGGGACGUGUUCAUCGAGAG |
| | GCCCAACGCCGUGGACACCUGCUACCCCUUCGACAUCCCCGACUACCAGAGCCUGAGG |
| | AGCAUCCUGGCCAACAACGGCAAGUUCGAGUUCAUCGCCGAGGAGUUCAGCUGGACCA |
| | CCGUGAAGCAGAACGGCAACGGGGGCAGCAACGCCUGCAAGAGGGGCCCCGGCAGCGG |
| | CUUCUUCAGCAGGCUGAACUGGCUGACCAAGAGCGACGGCAACGCCUACCCCCUGCAG |
| | AACCUGACCAAGGUGAACAACGGCGACUACGCCAGGCUGUACAUCUGGGGCGUGCACC |
| | ACCCCAGCACCGACGCCGAGCAGACCAACCUGUACAAGAACAACCCCGGCAGGGUGAC |
| | CGUGAGCACCAAGACCGACCAGACCAGCGUGGUGCCCAACAUCGGCAGCAGGCCCUGG |
| | GUGAGGGGCCAGAGCGGCAGGAUCAGCUUCUACUGGACCAUCGUGGAGCCCGGCGACC |
| | UGAUCGUGUUCAACACCAUCGGCAACCUGAUCGCCCCCAGGGGCCACUACAAGCUGAA |
| | CAGCCAGAAGAAGAGCACCAUCCUGAACACCGCCAUCCCCAUCGGCAGCUGC |
| 37 | Mu epitope in influenza A subtype H7 haemagglutinin head domain, nucleotide |
| | sequence |
| | UGCACCCAGGGCAAGAGGAGCACCGACCUGGGCCAGUGCGGCCUGCUGGGCACCCUGA |
| | UCGGCCCCCCCCAGUGCGACCAGUUCCUGGAGUUCAACGCCGACCUGAUCAUCGAGAG |
| | GAGGGAGGGCACCGACGUGUGCUACCCCGGCAAGUUCACCAACGAGGAGAGCCUGAGG |
| | CAGAUCCUGAGGGGCAGCGGCGGCAUCGACAAGGAGAGCAUGGGCUUCACCUACAGCG |
| | GCAUCAGGACCAACGGCGGCAGCAACGCCUGCAAGAGGGGCCCCGGCAGCGGCUUCUU |
| | CAGCAGGCUGAACUGGCUGCUGAGCAACAGCGACAACGCCGCCUUCCCCCAGAUGACC |
| | AAGAGCUACAGGAACCCCAGGAACAAGCCCGCCCUGAUCAUCUGGGGCGUGCACCACA |
| | GCGGCAGCACCACCGAGCAGACCAAGCUGUACGGCAGCGGCAACAAGCUGAUCACCGU |
| | GGGCAGCAGCAAGUACCAGCAGAGCUUCACCCCCAGCCCCGGCGCCAGGCCCCAGGUG |
| | AACGGCCAGAGCGGCAGGAUCGACUUCCACUGGCUGCUGCUGGACCCCAACGACACCG |
| | UGACCUUCACCUUCAACGGCGCCUUCAUCGCCCCCGACAGGGCCAGCUUCUUCAGGGG |
| | CGAGAGCCUGGGCGUGCAGAGCGACGUGCCCCUGGACAGCGGCUGCGAGGGCGACUGC |
| 38 | Mu epitope in influenza A subtype H10 haemagglutinin head domain, nucleotide |
| | sequence |
| | UGCAUGAAGAGCAGGAACUACAAGGACCUGGGCAACUGCCACCCCAUCGGCAUGAUCA |
| | UCGGCACCCCCGCCUGCGACCUGCACCUGACCGGCACCUGGGACACCCUGAUCGAGAG |
| | GAACAACAGCAUCGCCUACUGCUACCCCGGCGCCACCGUGAACGAGGAGGCCCUGAGG |
| | CAGAAGAUCAUGGAGAGCGGCGGCAUCGACAAGAUCAGCACCGGCUUCACCUACGGCA |
| | GCAGCAUCAACAGCAACGGCGGCAGCAACGCCUGCAAGAGGGGCCCCGGCAGCGGCUU |
| | CUUCAGCAGGCUGAACUGGCUGGUGAGCAAGAGCAAGGGCCAGAACUUCCCCCAGACC |
| | ACCAACACCUACAGGAACACCGACAGCGCCGAGCACCUGAUCAUCUGGGGCAUCCACC |
| | ACCCCAGCAGCACCCAGGAGAAGAACGACCUGUACGGCACCCAGAGCCUGAGCAUCAG |
| | CGUGGGCAGCAGCACCUACCAGAACAACUUCGUGCCCGUGGUGGGCGCCAGGCCCCAG |
| | GUGAACGGCCAGAGCGGCAGGAUCGACUUCCACUGGACCAUGGUGCAGCCCGGCGACA |
| | ACAUCACCUUCAGCCACAACGGCGGCCUGAUCGCCCCCAGCAGGGUGAGCAAGCUGAA |
| | GGGCAGGGGCCUGGGCAUCCAGAGCGGCGCCAGCGUGGACAACGACUGC |
| 39 | Mu epitope in influenza A subtype H15 haemagglutinin head domain, nucleotide |
| | sequence |
| | UGCACCAAGGGCAAGAAGGCCGUGGACCUGGGCAGCUGCGGCAUCCUGGGCACCAUCA |
| | UCGGCCCCCCCCAGUGCGACCUGCACCUGGAGUUCAAGGCCGACCUGAUCAUCGAGAG |
| | GAGGAACAGCAGCGACAUCUGCUACCCCGGCAGGUUCACCAACGAGGAGGCCCUGAGG |
| | CAGAUCAUCAGGGAGAGCGGCGGCAUCGACAAGGAGAGCAUGGGCUUCAGGUACAGCG |
| | GCAUCAGGACCAACGGCGGCAGCAACGCCUGCAAGAGGGGCCCCGGCAGCGGCUUCUU |
| | CAGCAGGCUGAACUGGCUGAGCAGCAGCAUGAACAACCAGGUGUUCCCCCAGCUGAAC |
| | CAGACCUACAGGAACACCAGGAAGGAGCCCGCCCUGAUCGUGUGGGGCGUGCACCACA |
| | GCAGCAGCCUGGACGAGCAGAACAAGCUGUACGGCACCGGCAACAAGCUGAUCACCGU |
| | GGGCAGCAGCAAGUACCAGCAGAGCUUCAGCCCCAGCCCCGGCGCCAGGCCCAAGGUG |
| | AACGGCCAGGCCGGCAGGAUCGACUUCCACUGGAUGCUGCUGGACCCCGGCGACACCG |
| | UGACCUUCACCUUCAACGGCGCCUUCAUCGCCCCCGACAGGGCCACCUUCCUGAGGAG |
| | CAACGCCCCCAGCGGCAUCGAGUACAACGGCAAGAGCCUGGGCAUCCAGAGCGACGCC |
| | CAGAUCGACGAGAGCUGC |
| 40 | Theta epitope in influenza A subtype H4 haemagglutinin head domain, |
| | nucleotide sequence |
| | UGCCCCAGCCCCCUGAGGCUGAUCGACGGCCAGGCCUGCGACAUCAUCAACGGCGCCC |
| | UGGGCAGCCCCGGCUGCGACCACCUGAACGGCGCCGAGUGGGACGUGUUCAUCGAGAG |
| | GCCCAACGCCGUGGACACCUGCUACCCCUUCGACAUCCCCGACUACCAGAGCCUGAGG |
| | AGCAUCCUGGCCAACAACGGCAAGUUCGAGUUCAUCGCCGAGGAGUUCAGCUGGACCA |
| | CCGUGAAGCAGAACGGCAGCGGCGGCAGCUACGCCUGCAAGAGGGGCAGCGACAACAG |
| | CUUCUUCAGCAGGCUGAACUGGCUGACCAAGAGCGACGGCAACGCCUACCCCCUGCAG |
| | AACCUGACCAAGGUGAACAACGGCGACUACGCCAGGCUGUACAUCUGGGGCGUGCACC |
| | ACCCCAGCACCGACGCCGAGCAGACCAACCUGUACAAGAACAACCCCGGCAGGGUGAC |
| | CGUGAGCACCAAGACCGACCAGACCAGCGUGGUGCCCAACAUCGGCAGCAGGCCCUGG |
| | GUGAGGGGCCAGAGCGGCAGGAUCAGCUUCUACUGGACCAUCGUGGAGCCCGGCGACC |
| | UGAUCGUGUUCAACACCAUCGGCAACCUGAUCGCCCCCAGGGGCCACUACAAGCUGAA |
| | CAGCCAGAAGAAGAGCACCAUCCUGAACACCGCCAUCCCCAUCGGCAGCUGC |
| 41 | Theta epitope in influenza A subtype H7 haemagglutinin head domain, |
| | nucleotide sequence |
| | UGCACCCAGGGCAAGAGGAGCACCGACCUGGGCCAGUGCGGCCUGCUGGGCACCCUGA |
| | UCGGCCCCCCCCAGUGCGACCAGUUCCUGGAGUUCAACGCCGACCUGAUCAUCGAGAG |
| | GAGGGAGGGCACCGACGUGUGCUACCCCGGCAAGUUCACCAACGAGGAGAGCCUGAGG |
| | CAGAUCCUGAGGGGCAGCGGCGGCAUCGACAAGGAGAGCAUGGGCUUCACCUACAGCG |
| | GCAUCAGGACCAGCGGCGGCAGCUACGCCUGCAAGAGGGGCAGCGACAACAGCUUCUU |
| | CAGCAGGCUGAACUGGCUGCUGAGCAACAGCGACAACGCCGCCUUCCCCCAGAUGACC |
| | AAGAGCUACAGGAACCCCAGGAACAAGCCCGCCCUGAUCAUCUGGGGCGUGCACCACA |
| | GCGGCAGCACCACCGAGCAGACCAAGCUGUACGGCAGCGGCAACAAGCUGAUCACCGU |
| | GGGCAGCAGCAAGUACCAGCAGAGCUUCACCCCCAGCCCCGGCGCCAGGCCCCAGGUG |
| | AACGGCCAGAGCGGCAGGAUCGACUUCCACUGGCUGCUGCUGGACCCCAACGACACCG |
| | UGACCUUCACCUUCAACGGCGCCUUCAUCGCCCCCGACAGGGCCAGCUUCUUCAGGGG |
| | CGAGAGCCUGGGCGUGCAGAGCGACGUGCCCCUGGACAGCGGCUGCGAGGGCGACUGC |
| 42 | Theta epitope in influenza A subtype H10 haemagglutinin head domain, |
| | nucleotide sequence |
| | UGCAUGAAGAGCAGGAACUACAAGGACCUGGGCAACUGCCACCCCAUCGGCAUGAUCA |
| | UCGGCACCCCCGCCUGCGACCUGCACCUGACCGGCACCUGGGACACCCUGAUCGAGAG |
| | GAACAACAGCAUCGCCUACUGCUACCCCGGCGCCACCGUGAACGAGGAGGCCCUGAGG |
| | CAGAAGAUCAUGGAGAGCGGCGGCAUCGACAAGAUCAGCACCGGCUUCACCUACGGCA |
| | GCAGCAUCAACAGCAGCGGCGGCAGCUACGCCUGCAAGAGGGGCAGCGACAACAGCUU |
| | CUUCAGCAGGCUGAACUGGCUGGUGAGCAAGAGCAAGGGCCAGAACUUCCCCCAGACC |
| | ACCAACACCUACAGGAACACCGACAGCGCCGAGCACCUGAUCAUCUGGGGCAUCCACC |
| | ACCCCAGCAGCACCCAGGAGAAGAACGACCUGUACGGCACCCAGAGCCUGAGCAUCAG |
| | CGUGGGCAGCAGCACCUACCAGAACAACUUCGUGCCCGUGGUGGGCGCCAGGCCCCAG |
| | GUGAACGGCCAGAGCGGCAGGAUCGACUUCCACUGGACCAUGGUGCAGCCCGGCGACA |
| | ACAUCACCUUCAGCCACAACGGCGGCCUGAUCGCCCCCAGCAGGGUGAGCAAGCUGAA |
| | GGGCAGGGGCCUGGGCAUCCAGAGCGGCGCCAGCGUGGACAACGACUGC |
| 43 | Theta epitope in influenza A subtype H15 haemagglutinin head domain, |
| | nucleotide sequence |
| | UGCACCAAGGGCAAGAAGGCCGUGGACCUGGGCAGCUGCGGCAUCCUGGGCACCAUCA |
| | UCGGCCCCCCCCAGUGCGACCUGCACCUGGAGUUCAAGGCCGACCUGAUCAUCGAGAG |
| | GAGGAACAGCAGCGACAUCUGCUACCCCGGCAGGUUCACCAACGAGGAGGCCCUGAGG |
| | CAGAUCAUCAGGGAGAGCGGCGGCAUCGACAAGGAGAGCAUGGGCUUCAGGUACAGCG |
| | GCAUCAGGACCAGCGGCGGCAGCUACGCCUGCAAGAGGGGCAGCGACAACAGCUUCUU |
| | CAGCAGGCUGAACUGGCUGAGCAGCAGCAUGAACAACCAGGUGUUCCCCCAGCUGAAC |
| | CAGACCUACAGGAACACCAGGAAGGAGCCCGCCCUGAUCGUGUGGGGCGUGCACCACA |
| | GCAGCAGCCUGGACGAGCAGAACAAGCUGUACGGCACCGGCAACAAGCUGAUCACCGU |
| | GGGCAGCAGCAAGUACCAGCAGAGCUUCAGCCCCAGCCCCGGCGCCAGGCCCAAGGUG |
| | AACGGCCAGGCCGGCAGGAUCGACUUCCACUGGAUGCUGCUGGACCCCGGCGACACCG |
| | UGACCUUCACCUUCAACGGCGCCUUCAUCGCCCCCGACAGGGCCACCUUCCUGAGGAG |
| | CAACGCCCCCAGCGGCAUCGAGUACAACGGCAAGAGCCUGGGCAUCCAGAGCGACGCC |
| | CAGAUCGACGAGAGCUGC |
| 44 | Rho epitope in influenza A subtype H4 haemagglutinin head |
| | domain, nucleotide sequence |
| | UGCCCCAGCCCCCUGAGGCUGAUCGACGGCCAGGCCUGCGACAUCAUCAACGGCGCCC |
| | UGGGCAGCCCCGGCUGCGACCACCUGAACGGCGCCGAGUGGGACGUGUUCAUCGAGAG |
| | GCCCAACGCCGUGGACACCUGCUACCCCUUCGACAUCCCCGACUACCAGAGCCUGAGG |
| | AGCAUCCUGGCCAACAACGGCAAGUUCGAGUUCAUCGCCGAGGAGUUCAGCUGGACCA |
| | CCGUGAAGCAGAACGGCAGCGGCGAGAGCUACGCCUGCAAGAGGGGCAGCGUGAAGAG |
| | CUUCUUCAGCAGGCUGAACUGGCUGACCAAGAGCGACGGCAACGCCUACCCCCUGCAG |
| | AACCUGACCAAGGUGAACAACGGCGACUACGCCAGGCUGUACAUCUGGGGCGUGCACC |
| | ACCCCAGCACCGACGCCGAGCAGACCAACCUGUACAAGAACAACCCCGGCAGGGUGAC |
| | CGUGAGCACCAAGACCGACCAGACCAGCGUGGUGCCCAACAUCGGCAGCAGGCCCUGG |
| | GUGAGGGGCCAGAGCGGCAGGAUCAGCUUCUACUGGACCAUCGUGGAGCCCGGCGACC |
| | UGAUCGUGUUCAACACCAUCGGCAACCUGAUCGCCCCCAGGGGCCACUACAAGCUGAA |
| | CAGCCAGAAGAAGAGCACCAUCCUGAACACCGCCAUCCCCAUCGGCAGCUGC |
| 45 | Rho epitope in influenza A subtype H7 haemagglutinin head |
| | domain, nucleotide sequence |
| | UGCACCCAGGGCAAGAGGAGCACCGACCUGGGCCAGUGCGGCCUGCUGGGCACCCUGA |
| | UCGGCCCCCCCCAGUGCGACCAGUUCCUGGAGUUCAACGCCGACCUGAUCAUCGAGAG |
| | GAGGGAGGGCACCGACGUGUGCUACCCCGGCAAGUUCACCAACGAGGAGAGCCUGAGG |
| | CAGAUCCUGAGGGGCAGCGGCGGCAUCGACAAGGAGAGCAUGGGCUUCACCUACAGCG |
| | GCAUCAGGACCAGCGGCGAGAGCUACGCCUGCAAGAGGGGCAGCGUGAAGAGCUUCUU |
| | CAGCAGGCUGAACUGGCUGCUGAGCAACAGCGACAACGCCGCCUUCCCCCAGAUGACC |
| | AAGAGCUACAGGAACCCCAGGAACAAGCCCGCCCUGAUCAUCUGGGGCGUGCACCACA |
| | GCGGCAGCACCACCGAGCAGACCAAGCUGUACGGCAGCGGCAACAAGCUGAUCACCGU |
| | GGGCAGCAGCAAGUACCAGCAGAGCUUCACCCCCAGCCCCGGCGCCAGGCCCCAGGUG |
| | AACGGCCAGAGCGGCAGGAUCGACUUCCACUGGCUGCUGCUGGACCCCAACGACACCG |
| | UGACCUUCACCUUCAACGGCGCCUUCAUCGCCCCCGACAGGGCCAGCUUCUUCAGGGG |
| | CGAGAGCCUGGGCGUGCAGAGCGACGUGCCCCUGGACAGCGGCUGCGAGGGCGACUGC |
| 46 | Rho epitope in influenza A subtype H10 haemagglutinin head |
| | domain, nucleotide sequence |
| | UGCAUGAAGAGCAGGAACUACAAGGACCUGGGCAACUGCCACCCCAUCGGCAUGAUCA |
| | UCGGCACCCCCGCCUGCGACCUGCACCUGACCGGCACCUGGGACACCCUGAUCGAGAG |
| | GAACAACAGCAUCGCCUACUGCUACCCCGGCGCCACCGUGAACGAGGAGGCCCUGAGG |
| | CAGAAGAUCAUGGAGAGCGGCGGCAUCGACAAGAUCAGCACCGGCUUCACCUACGGCA |
| | GCAGCAUCAACAGCAGCGGCGAGAGCUACGCCUGCAAGAGGGGCAGCGUGAAGAGCUU |
| | CUUCAGCAGGCUGAACUGGCUGGUGAGCAAGAGCAAGGGCCAGAACUUCCCCCAGACC |
| | ACCAACACCUACAGGAACACCGACAGCGCCGAGCACCUGAUCAUCUGGGGCAUCCACC |
| | ACCCCAGCAGCACCCAGGAGAAGAACGACCUGUACGGCACCCAGAGCCUGAGCAUCAG |
| | CGUGGGCAGCAGCACCUACCAGAACAACUUCGUGCCCGUGGUGGGCGCCAGGCCCCAG |
| | GUGAACGGCCAGAGCGGCAGGAUCGACUUCCACUGGACCAUGGUGCAGCCCGGCGACA |
| | ACAUCACCUUCAGCCACAACGGCGGCCUGAUCGCCCCCAGCAGGGUGAGCAAGCUGAA |
| | GGGCAGGGGCCUGGGCAUCCAGAGCGGCGCCAGCGUGGACAACGACUGC |
| 47 | Rho epitope in influenza A subtype H15 haemagglutinin head |
| | domain, nucleotide sequence |
| | UGCACCAAGGGCAAGAAGGCCGUGGACCUGGGCAGCUGCGGCAUCCUGGGCACCAUCA |
| | UCGGCCCCCCCCAGUGCGACCUGCACCUGGAGUUCAAGGCCGACCUGAUCAUCGAGAG |
| | GAGGAACAGCAGCGACAUCUGCUACCCCGGCAGGUUCACCAACGAGGAGGCCCUGAGG |
| | CAGAUCAUCAGGGAGAGCGGCGGCAUCGACAAGGAGAGCAUGGGCUUCAGGUACAGCG |
| | GCAUCAGGACCAGCGGCGAGAGCUACGCCUGCAAGAGGGGCAGCGUGAAGAGCUUCUU |
| | CAGCAGGCUGAACUGGCUGAGCAGCAGCAUGAACAACCAGGUGUUCCCCCAGCUGAAC |
| | CAGACCUACAGGAACACCAGGAAGGAGCCCGCCCUGAUCGUGUGGGGCGUGCACCACA |
| | GCAGCAGCCUGGACGAGCAGAACAAGCUGUACGGCACCGGCAACAAGCUGAUCACCGU |
| | GGGCAGCAGCAAGUACCAGCAGAGCUUCAGCCCCAGCCCCGGCGCCAGGCCCAAGGUG |
| | AACGGCCAGGCCGGCAGGAUCGACUUCCACUGGAUGCUGCUGGACCCCGGCGACACCG |
| | UGACCUUCACCUUCAACGGCGCCUUCAUCGCCCCCGACAGGGCCACCUUCCUGAGGAG |
| | CAACGCCCCCAGCGGCAUCGAGUACAACGGCAAGAGCCUGGGCAUCCAGAGCGACGCC |
| | CAGAUCGACGAGAGCUGC |
| 48 | Sigma epitope in influenza A subtype H4 haemagglutinin head domain, |
| | nucleotide sequence |
| | UGCCCCAGCCCCCUGAGGCUGAUCGACGGCCAGGCCUGCGACAUCAUCAACGGCGCCC |
| | UGGGCAGCCCCGGCUGCGACCACCUGAACGGCGCCGAGUGGGACGUGUUCAUCGAGAG |
| | GCCCAACGCCGUGGACACCUGCUACCCCUUCGACAUCCCCGACUACCAGAGCCUGAGG |
| | AGCAUCCUGGCCAACAACGGCAAGUUCGAGUUCAUCGCCGAGGAGUUCAGCUGGACCA |
| | CCGUGAAGCAGAACGGCAACGGCACCAGCAGCGCCUGCAAGAGGAGGAGCAACAAGAG |
| | CUUCUUCAGCAGGCUGAACUGGCUGACCAAGAGCGACGGCAACGCCUACCCCCUGCAG |
| | AACCUGACCAAGGUGAACAACGGCGACUACGCCAGGCUGUACAUCUGGGGCGUGCACC |
| | ACCCCAGCACCGACGCCGAGCAGACCAACCUGUACAAGAACAACCCCGGCAGGGUGAC |
| | CGUGAGCACCAAGACCGACCAGACCAGCGUGGUGCCCAACAUCGGCAGCAGGCCCUGG |
| | GUGAGGGGCCAGAGCGGCAGGAUCAGCUUCUACUGGACCAUCGUGGAGCCCGGCGACC |
| | UGAUCGUGUUCAACACCAUCGGCAACCUGAUCGCCCCCAGGGGCCACUACAAGCUGAA |
| | CAGCCAGAAGAAGAGCACCAUCCUGAACACCGCCAUCCCCAUCGGCAGCUGC |
| 49 | Sigma epitope in influenza A subtype H7 haemagglutinin head domain, |
| | nucleotide sequence |
| | UGCACCCAGGGCAAGAGGAGCACCGACCUGGGCCAGUGCGGCCUGCUGGGCACCCUGA |
| | UCGGCCCCCCCCAGUGCGACCAGUUCCUGGAGUUCAACGCCGACCUGAUCAUCGAGAG |
| | GAGGGAGGGCACCGACGUGUGCUACCCCGGCAAGUUCACCAACGAGGAGAGCCUGAGG |
| | CAGAUCCUGAGGGGCAGCGGCGGCAUCGACAAGGAGAGCAUGGGCUUCACCUACAGCG |
| | GCAUCAGGACCAACGGCACCAGCAGCGCCUGCAAGAGGAGGAGCAACAAGAGCUUCUU |
| | CAGCAGGCUGAACUGGCUGCUGAGCAACAGCGACAACGCCGCCUUCCCCCAGAUGACC |
| | AAGAGCUACAGGAACCCCAGGAACAAGCCCGCCCUGAUCAUCUGGGGCGUGCACCACA |
| | GCGGCAGCACCACCGAGCAGACCAAGCUGUACGGCAGCGGCAACAAGCUGAUCACCGU |
| | GGGCAGCAGCAAGUACCAGCAGAGCUUCACCCCCAGCCCCGGCGCCAGGCCCCAGGUG |
| | AACGGCCAGAGCGGCAGGAUCGACUUCCACUGGCUGCUGCUGGACCCCAACGACACCG |
| | UGACCUUCACCUUCAACGGCGCCUUCAUCGCCCCCGACAGGGCCAGCUUCUUCAGGGG |
| | CGAGAGCCUGGGCGUGCAGAGCGACGUGCCCCUGGACAGCGGCUGCGAGGGCGACUGC |
| 50 | Sigma epitope in influenza A subtype H10 haemagglutinin head domain, |
| | nucleotide sequence |
| | UGCAUGAAGAGCAGGAACUACAAGGACCUGGGCAACUGCCACCCCAUCGGCAUGAUCA |
| | UCGGCACCCCCGCCUGCGACCUGCACCUGACCGGCACCUGGGACACCCUGAUCGAGAG |
| | GAACAACAGCAUCGCCUACUGCUACCCCGGCGCCACCGUGAACGAGGAGGCCCUGAGG |
| | CAGAAGAUCAUGGAGAGCGGCGGCAUCGACAAGAUCAGCACCGGCUUCACCUACGGCA |
| | GCAGCAUCAACAGCAACGGCACCAGCAGCGCCUGCAAGAGGAGGAGCAACAAGAGCUU |
| | CUUCAGCAGGCUGAACUGGCUGGUGAGCAAGAGCAAGGGCCAGAACUUCCCCCAGACC |
| | ACCAACACCUACAGGAACACCGACAGCGCCGAGCACCUGAUCAUCUGGGGCAUCCACC |
| | ACCCCAGCAGCACCCAGGAGAAGAACGACCUGUACGGCACCCAGAGCCUGAGCAUCAG |
| | CGUGGGCAGCAGCACCUACCAGAACAACUUCGUGCCCGUGGUGGGCGCCAGGCCCCAG |
| | GUGAACGGCCAGAGCGGCAGGAUCGACUUCCACUGGACCAUGGUGCAGCCCGGCGACA |
| | ACAUCACCUUCAGCCACAACGGCGGCCUGAUCGCCCCCAGCAGGGUGAGCAAGCUGAA |
| | GGGCAGGGGCCUGGGCAUCCAGAGCGGCGCCAGCGUGGACAACGACUGC |
| 51 | Sigma epitope in influenza A subtype H15 haemagglutinin head domain, |
| | nucleotide sequence |
| | UGCACCAAGGGCAAGAAGGCCGUGGACCUGGGCAGCUGCGGCAUCCUGGGCACCAUCA |
| | UCGGCCCCCCCCAGUGCGACCUGCACCUGGAGUUCAAGGCCGACCUGAUCAUCGAGAG |
| | GAGGAACAGCAGCGACAUCUGCUACCCCGGCAGGUUCACCAACGAGGAGGCCCUGAGG |
| | CAGAUCAUCAGGGAGAGCGGCGGCAUCGACAAGGAGAGCAUGGGCUUCAGGUACAGCG |
| | GCAUCAGGACCAACGGCACCAGCAGCGCCUGCAAGAGGAGGAGCAACAAGAGCUUCUU |
| | CAGCAGGCUGAACUGGCUGAGCAGCAGCAUGAACAACCAGGUGUUCCCCCAGCUGAAC |
| | CAGACCUACAGGAACACCAGGAAGGAGCCCGCCCUGAUCGUGUGGGGCGUGCACCACA |
| | GCAGCAGCCUGGACGAGCAGAACAAGCUGUACGGCACCGGCAACAAGCUGAUCACCGU |
| | GGGCAGCAGCAAGUACCAGCAGAGCUUCAGCCCCAGCCCCGGCGCCAGGCCCAAGGUG |
| | AACGGCCAGGCCGGCAGGAUCGACUUCCACUGGAUGCUGCUGGACCCCGGCGACACCG |
| | UGACCUUCACCUUCAACGGCGCCUUCAUCGCCCCCGACAGGGCCACCUUCCUGAGGAG |
| | CAACGCCCCCAGCGGCAUCGAGUACAACGGCAAGAGCCUGGGCAUCCAGAGCGACGCC |
| | CAGAUCGACGAGAGCUGC |
| 52 | Tau epitope in influenza A subtype H4 haemagglutinin head |
| | domain, nucleotide sequence |
| | UGCCCCAGCCCCCUGAGGCUGAUCGACGGCCAGGCCUGCGACAUCAUCAACGGCGCCC |
| | UGGGCAGCCCCGGCUGCGACCACCUGAACGGCGCCGAGUGGGACGUGUUCAUCGAGAG |
| | GCCCAACGCCGUGGACACCUGCUACCCCUUCGACAUCCCCGACUACCAGAGCCUGAGG |
| | AGCAUCCUGGCCAACAACGGCAAGUUCGAGUUCAUCGCCGAGGAGUUCAGCUGGACCA |
| | CCGUGAAGCAGAACGGCAACGGCACCAGCAGCGCCUGCAAGAGGAGGAGCAACAACAG |
| | CUUCUUCAGCAGGCUGAACUGGCUGACCAAGAGCGACGGCAACGCCUACCCCCUGCAG |
| | AACCUGACCAAGGUGAACAACGGCGACUACGCCAGGCUGUACAUCUGGGGCGUGCACC |
| | ACCCCAGCACCGACGCCGAGCAGACCAACCUGUACAAGAACAACCCCGGCAGGGUGAC |
| | CGUGAGCACCAAGACCGACCAGACCAGCGUGGUGCCCAACAUCGGCAGCAGGCCCUGG |
| | GUGAGGGGCCAGAGCGGCAGGAUCAGCUUCUACUGGACCAUCGUGGAGCCCGGCGACC |
| | UGAUCGUGUUCAACACCAUCGGCAACCUGAUCGCCCCCAGGGGCCACUACAAGCUGAA |
| | CAGCCAGAAGAAGAGCACCAUCCUGAACACCGCCAUCCCCAUCGGCAGCUGC |
| 53 | Tau epitope in influenza A subtype H7 haemagglutinin head |
| | domain, nucleotide sequence |
| | UGCACCCAGGGCAAGAGGAGCACCGACCUGGGCCAGUGCGGCCUGCUGGGCACCCUGA |
| | UCGGCCCCCCCCAGUGCGACCAGUUCCUGGAGUUCAACGCCGACCUGAUCAUCGAGAG |
| | GAGGGAGGGCACCGACGUGUGCUACCCCGGCAAGUUCACCAACGAGGAGAGCCUGAGG |
| | CAGAUCCUGAGGGGCAGCGGCGGCAUCGACAAGGAGAGCAUGGGCUUCACCUACAGCG |
| | GCAUCAGGACCAACGGCACCAGCAGCGCCUGCAAGAGGAGGAGCAACAACAGCUUCUU |
| | CAGCAGGCUGAACUGGCUGCUGAGCAACAGCGACAACGCCGCCUUCCCCCAGAUGACC |
| | AAGAGCUACAGGAACCCCAGGAACAAGCCCGCCCUGAUCAUCUGGGGCGUGCACCACA |
| | GCGGCAGCACCACCGAGCAGACCAAGCUGUACGGCAGCGGCAACAAGCUGAUCACCGU |
| | GGGCAGCAGCAAGUACCAGCAGAGCUUCACCCCCAGCCCCGGCGCCAGGCCCCAGGUG |
| | AACGGCCAGAGCGGCAGGAUCGACUUCCACUGGCUGCUGCUGGACCCCAACGACACCG |
| | UGACCUUCACCUUCAACGGCGCCUUCAUCGCCCCCGACAGGGCCAGCUUCUUCAGGGG |
| | CGAGAGCCUGGGCGUGCAGAGCGACGUGCCCCUGGACAGCGGCUGCGAGGGCGACUGC |
| 54 | Tau epitope in influenza A subtype H10 haemagglutinin head |
| | domain, nucleotide sequence |
| | UGCAUGAAGAGCAGGAACUACAAGGACCUGGGCAACUGCCACCCCAUCGGCAUGAUCA |
| | UCGGCACCCCCGCCUGCGACCUGCACCUGACCGGCACCUGGGACACCCUGAUCGAGAG |
| | GAACAACAGCAUCGCCUACUGCUACCCCGGCGCCACCGUGAACGAGGAGGCCCUGAGG |
| | CAGAAGAUCAUGGAGAGCGGCGGCAUCGACAAGAUCAGCACCGGCUUCACCUACGGCA |
| | GCAGCAUCAACAGCAACGGCACCAGCAGCGCCUGCAAGAGGAGGAGCAACAACAGCUU |
| | CUUCAGCAGGCUGAACUGGCUGGUGAGCAAGAGCAAGGGCCAGAACUUCCCCCAGACC |
| | ACCAACACCUACAGGAACACCGACAGCGCCGAGCACCUGAUCAUCUGGGGCAUCCACC |
| | ACCCCAGCAGCACCCAGGAGAAGAACGACCUGUACGGCACCCAGAGCCUGAGCAUCAG |
| | CGUGGGCAGCAGCACCUACCAGAACAACUUCGUGCCCGUGGUGGGCGCCAGGCCCCAG |
| | GUGAACGGCCAGAGCGGCAGGAUCGACUUCCACUGGACCAUGGUGCAGCCCGGCGACA |
| | ACAUCACCUUCAGCCACAACGGCGGCCUGAUCGCCCCCAGCAGGGUGAGCAAGCUGAA |
| | GGGCAGGGGCCUGGGCAUCCAGAGCGGCGCCAGCGUGGACAACGACUGC |
| 55 | Tau epitope in influenza A subtype H15 haemagglutinin head |
| | domain, nucleotide sequence |
| | UGCACCAAGGGCAAGAAGGCCGUGGACCUGGGCAGCUGCGGCAUCCUGGGCACCAUCA |
| | UCGGCCCCCCCCAGUGCGACCUGCACCUGGAGUUCAAGGCCGACCUGAUCAUCGAGAG |
| | GAGGAACAGCAGCGACAUCUGCUACCCCGGCAGGUUCACCAACGAGGAGGCCCUGAGG |
| | CAGAUCAUCAGGGAGAGCGGCGGCAUCGACAAGGAGAGCAUGGGCUUCAGGUACAGCG |
| | GCAUCAGGACCAACGGCACCAGCAGCGCCUGCAAGAGGAGGAGCAACAACAGCUUCUU |
| | CAGCAGGCUGAACUGGCUGAGCAGCAGCAUGAACAACCAGGUGUUCCCCCAGCUGAAC |
| | CAGACCUACAGGAACACCAGGAAGGAGCCCGCCCUGAUCGUGUGGGGCGUGCACCACA |
| | GCAGCAGCCUGGACGAGCAGAACAAGCUGUACGGCACCGGCAACAAGCUGAUCACCGU |
| | GGGCAGCAGCAAGUACCAGCAGAGCUUCAGCCCCAGCCCCGGCGCCAGGCCCAAGGUG |
| | AACGGCCAGGCCGGCAGGAUCGACUUCCACUGGAUGCUGCUGGACCCCGGCGACACCG |
| | UGACCUUCACCUUCAACGGCGCCUUCAUCGCCCCCGACAGGGCCACCUUCCUGAGGAG |
| | CAACGCCCCCAGCGGCAUCGAGUACAACGGCAAGAGCCUGGGCAUCCAGAGCGACGCC |
| | CAGAUCGACGAGAGCUGC |
| 56 | Backbone influenza A subtype H4 haemagglutinin head domain, nucleotide |
| | sequence |
| | UGCCCCAGCCCCCUGAGGCUGAUCGACGGCCAGGCCUGCGACAUCAUCAACGGCGCCC |
| | UGGGCAGCCCCGGCUGCGACCACCUGAACGGCGCCGAGUGGGACGUGUUCAUCGAGAG |
| | GCCCAACGCCGUGGACACCUGCUACCCCUUCGACAUCCCCGACUACCAGAGCCUGAGG |
| | AGCAUCCUGGCCAACAACGGCAAGUUCGAGUUCAUCGCCGAGGAGUUCAGCUGGACCA |
| | CCGUGAAGCAGAACGGCAAGAGCGGCGCCUGCAAGAGGGCCAACGUGAACGACUUCUU |
| | CAACAGGCUGAACUGGCUGACCAAGAGCGACGGCAACGCCUACCCCCUGCAGAACCUG |
| | ACCAAGGUGAACAACGGCGACUACGCCAGGCUGUACAUCUGGGGCGUGCACCACCCCA |
| | GCACCGACGCCGAGCAGACCAACCUGUACAAGAACAACCCCGGCAGGGUGACCGUGAG |
| | CACCAAGACCGACCAGACCAGCGUGGUGCCCAACAUCGGCAGCAGGCCCUGGGUGAGG |
| | GGCCAGAGCGGCAGGAUCAGCUUCUACUGGACCAUCGUGGAGCCCGGCGACCUGAUCG |
| | UGUUCAACACCAUCGGCAACCUGAUCGCCCCCAGGGGCCACUACAAGCUGAACAGCCA |
| | GAAGAAGAGCACCAUCCUGAACACCGCCAUCCCCAUCGGCAGCUGC |
| 57 | Backbone influenza A subtype H7 haemagglutinin head domain, nucleotide |
| | sequence |
| | UGCACCCAGGGCAAGAGGAGCACCGACCUGGGCCAGUGCGGCCUGCUGGGCACCCUGA |
| | UCGGCCCCCCCCAGUGCGACCAGUUCCUGGAGUUCAACGCCGACCUGAUCAUCGAGAG |
| | GAGGGAGGGCACCGACGUGUGCUACCCCGGCAAGUUCACCAACGAGGAGAGCCUGAGG |
| | CAGAUCCUGAGGGGCAGCGGCGGCAUCGACAAGGAGAGCAUGGGCUUCACCUACAGCG |
| | GCAUCAGGACCAACGGCGCCACCAGCGCCUGCAGGAGGAGCGGCAGCAGCUUCUACGC |
| | CGAGAUGAAGUGGCUGCUGAGCAACAGCGACAACGCCGCCUUCCCCCAGAUGACCAAG |
| | AGCUACAGGAACCCCAGGAACAAGCCCGCCCUGAUCAUCUGGGGCGUGCACCACAGCG |
| | GCAGCACCACCGAGCAGACCAAGCUGUACGGCAGCGGCAACAAGCUGAUCACCGUGGG |
| | CAGCAGCAAGUACCAGCAGAGCUUCACCCCCAGCCCCGGCGCCAGGCCCCAGGUGAAC |
| | GGCCAGAGCGGCAGGAUCGACUUCCACUGGCUGCUGCUGGACCCCAACGACACCGUGA |
| | CCUUCACCUUCAACGGCGCCUUCAUCGCCCCCGACAGGGCCAGCUUCUUCAGGGGCGA |
| | GAGCCUGGGCGUGCAGAGCGACGUGCCCCUGGACAGCGGCUGCGAGGGCGACUGC |
| 58 | Backbone influenza A subtype H10 haemagglutinin head domain, nucleotide |
| | sequence |
| | UGCAUGAAGAGCAGGAACUACAAGGACCUGGGCAACUGCCACCCCAUCGGCAUGAUCA |
| | UCGGCACCCCCGCCUGCGACCUGCACCUGACCGGCACCUGGGACACCCUGAUCGAGAG |
| | GAACAACAGCAUCGCCUACUGCUACCCCGGCGCCACCGUGAACGAGGAGGCCCUGAGG |
| | CAGAAGAUCAUGGAGAGCGGCGGCAUCGACAAGAUCAGCACCGGCUUCACCUACGGCA |
| | GCAGCAUCAACAGCGCCGGCACCACCGCCGCCUGCAUGAGGAACGGCGGCAACAGCUU |
| | CUACGCCGAGCUGAAGUGGCUGGUGAGCAAGAGCAAGGGCCAGAACUUCCCCCAGACC |
| | ACCAACACCUACAGGAACACCGACAGCGCCGAGCACCUGAUCAUCUGGGGCAUCCACC |
| | ACCCCAGCAGCACCCAGGAGAAGAACGACCUGUACGGCACCCAGAGCCUGAGCAUCAG |
| | CGUGGGCAGCAGCACCUACCAGAACAACUUCGUGCCCGUGGUGGGCGCCAGGCCCCAG |
| | GUGAACGGCCAGAGCGGCAGGAUCGACUUCCACUGGACCAUGGUGCAGCCCGGCGACA |
| | ACAUCACCUUCAGCCACAACGGCGGCCUGAUCGCCCCCAGCAGGGUGAGCAAGCUGAA |
| | GGGCAGGGGCCUGGGCAUCCAGAGCGGCGCCAGCGUGGACAACGACUGC |
| 59 | Backbone influenza A subtype H15 haemagglutinin head domain, nucleotide |
| | sequence |
| | UGCACCAAGGGCAAGAAGGCCGUGGACCUGGGCAGCUGCGGCAUCCUGGGCACCAUCA |
| | UCGGCCCCCCCCAGUGCGACCUGCACCUGGAGUUCAAGGCCGACCUGAUCAUCGAGAG |
| | GAGGAACAGCAGCGACAUCUGCUACCCCGGCAGGUUCACCAACGAGGAGGCCCUGAGG |
| | CAGAUCAUCAGGGAGAGCGGCGGCAUCGACAAGGAGAGCAUGGGCUUCAGGUACAGCG |
| | GCAUCAGGACCGACGGCGCCACCAGCGCCUGCAAGAGGACCGUGAGCAGCUUCUACAG |
| | CGAGAUGAAGUGGCUGAGCAGCAGCAUGAACAACCAGGUGUUCCCCCAGCUGAACCAG |
| | ACCUACAGGAACACCAGGAAGGAGCCCGCCCUGAUCGUGUGGGGCGUGCACCACAGCA |
| | GCAGCCUGGACGAGCAGAACAAGCUGUACGGCACCGGCAACAAGCUGAUCACCGUGGG |
| | CAGCAGCAAGUACCAGCAGAGCUUCAGCCCCAGCCCCGGCGCCAGGCCCAAGGUGAAC |
| | GGCCAGGCCGGCAGGAUCGACUUCCACUGGAUGCUGCUGGACCCCGGCGACACCGUGA |
| | CCUUCACCUUCAACGGCGCCUUCAUCGCCCCCGACAGGGCCACCUUCCUGAGGAGCAA |
| | CGCCCCCAGCGGCAUCGAGUACAACGGCAAGAGCCUGGGCAUCCAGAGCGACGCCCAG |
| | AUCGACGAGAGCUGC |

1. (canceled)

2. A polypeptide which comprises a first region of contiguous amino acids, wherein:
(a) the amino acid sequence of the first region has at least 80% sequence identity to an influenza A haemagglutinin head domain; and
(b) the first region has the following specified amino acids at positions corresponding to the positions given in SEQ ID NO: 31:
position 155 is C
position 156 is I or K
position 157 is R
position 158 is G or R
position 159 is P or S
position 160 is a non-polar or uncharged amino acid or is D or K
position 161 is an uncharged amino acid or is K
position 162 is an uncharged amino acid
position 163 is F
position 164 is F
position 165 is S
position 166 is R
position 167 is L
position 168 is N and
position 169 is W.

3. A polypeptide as claimed in claim 2, wherein:
(b) the first region of the polypeptide has the following specified amino acids at positions which correspond to the positions in SEQ ID NO:31:
position 155 is C
position 156 is I or K
position 157 is R
position 158 is G or R
position 159 is P or S
position 160 is I, V, S, N, G, D or K
position 161 is S, N or K
position 162 is G or S
position 163 is F
position 164 is F
position 165 is S
position 166 is R
position 167 is L
position 168 is N and
position 169 is W.

4. A polypeptide as claimed in claim 3, wherein:
(A) (b) the first region of the polypeptide has the following specified amino acids at positions which correspond to the positions in SEQ ID NO:31:
position 155 is C
position 156 is I
position 157 is R
position 158 is G or R,
position 159 is S
position 160 is N or S,
position 161 is N or S,
position 162 is S
position 163 is F
position 164 is F
position 165 is S
position 166 is R
position 167 is L
position 168 is N and
position 169 is W; or
(B) (b) the first region of the polypeptide has the following specified amino acids at positions which correspond to the positions in SEQ ID NO:31:
position 155 is C
position 156 is I
position 157 is R
position 158 is R
position 159 is S
position 160 is S
position 161 is S
position 162 is S
position 163 is F
position 164 is E
position 165 is S
position 166 is R
position 167 is L
position 168 is N and
position 169 is W; or
(C) the first region of one polypeptide comprises or consists of the amino acid sequence given in SEQ ID NO: 21.

5-6. (canceled)

7. A polypeptide as claimed in claim 3, wherein:
(A) (b) the first region of the polypeptide has the following specified amino acids at positions which correspond to the positions in SEQ ID NO:31:
position 155 is C
position 156 is K
position 157 is R
position 158 is R
position 159 is S
position 160 is N
position 161 is N
position 162 is S
position 163 is F
position 164 is F
position 165 is S
position 166 is R
position 167 is L
position 168 is N and
position 169 is W; or
(B) the first region of one polypeptide comprises or consists of the amino acid sequence given in SEQ ID NO: 22.

8. (canceled)

9. A polypeptide as claimed in claim 3, wherein:
(A) (b) the first region of the polypeptide has the following specified amino acids at positions which correspond to the positions in SEQ ID NO:31:
position 155 is C
position 156 is K
position 157 is R
position 158 is R
position 159 is S
position 160 is D, I, N or V,
position 161 is K
position 162 is S
position 163 is F
position 164 is F
position 165 is S
position 166 is R
position 167 is L
position 168 is N and
position 169 is W; or
(B) (b) the first region of the polypeptide has the following specified amino acids at positions which correspond to the positions in SEQ ID NO:31:
position 155 is C
position 156 is K
position 157 is R
position 158 is R
position 159 is S
position 160 is N
position 161 is K
position 162 is S
position 163 is F
position 164 is F
position 165 is S
position 166 is R
position 167 is L
position 168 is N and
position 169 is W; or
(C) the first region of one polypeptide comprises or consists of the amino acid sequence given in SEQ ID NO: 23.

10-11. (canceled)

12. A polypeptide as claimed in claim 3, wherein:
(A) (b) the first region of the polypeptide has the following specified amino acids at positions which correspond to the positions in SEQ ID NO:31:
position 155 is C
position 156 is K
position 157 is R
position 158 is G
position 159 is S
position 160 is V
position 161 is K
position 162 is S
position 163 is F
position 164 is F
position 165 is S
position 166 is R
position 167 is L
position 168 is N and
position 169 is W; or
(B) the first region of one polypeptide comprises or consists of the amino acid sequence given in SEQ ID NO: 24.

13. (canceled)

14. A polypeptide as claimed in claim 3, wherein:
(A) (b) the first region of the polypeptide has the following specified amino acids at positions which correspond to the positions in SEQ ID NO: 31:
position 155 is C
position 156 is K
position 157 is R
position 158 is G
position 159 is P or S,
position 160 is G or V,
position 161 is N or S,
position 162 is G or S,
position 163 is F
position 164 is F
position 165 is S
position 166 is R
position 167 is L
position 168 is N and
position 169 is W; or
(B) (b) the first region of the polypeptide has the following specified amino acids at positions which correspond to the positions in SEQ ID NO: 31:
position 155 is C
position 156 is K
position 157 is R
position 158 is G
position 159 is S
position 160 is V
position 161 is N
position 162 is S
position 163 is F
position 164 is F
position 165 is S
position 166 is R
position 167 is L
position 168 is N and
position 169 is W; or
(C) the first region of one polypeptide comprises or consists of the amino acid sequence given in SEQ ID NO: 25.

15-16. (canceled)

17. A polypeptide as claimed in claim 3, wherein:
(A) (b) the first region of the polypeptide has the following specified amino acids at positions which correspond to the positions in SEQ ID NO: 31
position 155 is C
position 156 is K
position 157 is R
position 158 is G
position 159 is P or S
position 160 is D
position 161 is K, N or S,
position 162 is G or S,
position 163 is F
position 164 is F
position 165 is S
position 166 is R
position 167 is L
position 168 is N and
position 169 is W; or
(B) (b) the first region of the polypeptide has the following specified amino acids at positions which correspond to the positions in SEQ ID NO: 31:
position 155 is C
position 156 is K
position 157 is R
position 158 is G
position 159 is S
position 160 is D
position 161 is N
position 162 is S
position 163 is F
position 164 is F
position 165 is S
position 166 is R
position 167 is L
position 168 is N
position 169 is W; or
(C) the first region of one polypeptide comprises or consists of the amino acid sequence given in SEQ ID NO: 26.

18-31. (canceled)

32. A polypeptide as claimed in claim 2 wherein:
(A) (a) the amino acid sequence of the first region of the polypeptide has at least 80% sequence identity to an influenza A subtype H1, H2, H3, H4, H5, H6, H7, H8, H9, H10, H11, H12, H13, H14, H15, H16, H17 or H18 haemagglutinin head domain; or
(B) (a) the amino acid sequence of the first region of the polypeptide has at least 80% sequence identity to an influenza A subtype H4, H7, H10, H14 or H15 haemagglutinin head domain; or
(C) (a) the amino acid sequence of the first region of the polypeptide has at least 80% sequence identity to the head domain as given in any one of SEQ ID NOs: 1-5.

33-34. (canceled)

35. A polypeptide as claimed in claim 2, wherein:
(i) the polypeptide additionally comprises a stretch of contiguous amino acids which are derived from a haemagglutinin N-terminal stalk region; and/or
(ii) the polypeptide additionally comprises a stretch of contiguous amino acids which are derived from a haemagglutinin C-terminal stalk region.

36. A polypeptide as claimed in claim 2, wherein the polypeptide is 280-300 amino acids in length.

37. A nucleic acid molecule or a mRNA which codes for a polypeptide as claimed in claim 2.

38. A vector or plasmid comprising a nucleic acid molecule or a mRNA as claimed in claim 37, wherein the vector is an expression vector or a viral vector.

39. A virus-like particle (VLP) comprising one or polypeptides as claimed in claim 2.

40. A pharmaceutical composition comprising a polypeptide as claimed in claim 2 together with a pharmaceutically-acceptable carrier, excipient, diluent or adjuvant.

41. A pharmaceutical composition, comprising 2, 3, 4, 5, 6, 7, 8, 9 or 10 polypeptides as claimed in claim 2 the first regions of which all have different amino acid sequences: or 2, 3, 4, 5, 6, 7, 8, 9 or 10 mRNAs encoding said polypeptides.

42-44. (canceled)

45. A pharmaceutical composition as claimed in claim 40, wherein the composition comprises one or more hetero-trimers of three different polypeptides or homo-trimers of the same polypeptides.

46-49. (canceled)

50. A method of:
(i) preventing or treating influenza infection in a subject: or
(ii) inducing a T-cell or B-cell response to an influenza antigen in a subject, the method comprising administering an effective amount of a first polypeptide as claimed in claim 2, to a subject in need thereof.

51. The method as claimed in claim 50, wherein the method comprises the additional steps of:
(ii) administering a boost with a second polypeptide which comprises a first region of contiguous amino acids, wherein:
(a) the amino acid sequence of the first region has at least 80% sequence identity to an influenza A haemagglutinin head domain; and
(b) the first region has the following specified amino acids at positions corresponding to the positions given in SEQ ID NO: 31:
position 155 is C
position 156 is I or K
position 157 is R
position 158 is G or R
position 159 is P or S
position 160 is a non-polar or uncharged amino acid or is D or K
position 161 is an uncharged amino acid or is K
position 162 is an uncharged amino acid
position 163 is F
position 164 is F
position 165 is S
position 166 is R
position 167 is L
position 168 is N and
position 169 is W to the subject; and optionally also
(iii) administering a boost with a third polypeptide which comprises a first region of contiguous amino acids, wherein:
(a) the amino acid sequence of the first region has at least 80% sequence identity to an influenza A haemagglutinin head domain; and
(b) the first region has the following specified amino acids at positions corresponding to the positions given in SEQ ID NO: 31:
position 155 is C
position 156 is I or K
position 157 is R
position 158 is G or R
position 159 is P or S
position 160 is a non-polar or uncharged amino acid or is D or K
position 161 is an uncharged amino acid or is K
position 162 is an uncharged amino acid
position 163 is F
position 164 is F
position 165 is S
position 166 is R
position 167 is L
position 168 is N and
position 169 is W to the subject,
wherein the second and third polypeptides are different from each other and different from the first polypeptide.

52. A pharmaceutical composition comprising a VLP as claimed in claim 39, together with a pharmaceutically-acceptable carrier, excipient, diluent or adjuvant.
